SEQUENCE LISTING (6 sequences)

SEQ ID NO: 1 - DNA, 26 bp, synthetic construct
  source 1..26: mol_type = other DNA
  misc_difference 1: note = Y is C or T
  misc_difference 3: note = Y is C or T
  misc_difference 14: note = Y is C or T
  misc_difference 24: note = Y is C or T
yayttgggggtagytaaagtgaaytg

SEQ ID NO: 2 - DNA, 28 bp, synthetic construct
  source 1..28: mol_type = other DNA
  misc_difference 9: note = R is A or G
tcctacaarcattaattaattaacacac

SEQ ID NO: 3 - DNA, 27 bp, synthetic construct
  source 1..27: mol_type = other DNA
  misc_difference 7: note = Y is C or T
  misc_difference 14: note = Y is C or T
  misc_difference 19: note = Y is C or T
  misc_difference 23: note = Y is C or T
ataaaayttaaaaytttayagtyagag

SEQ ID NO: 4 - DNA, 25 bp, synthetic construct
  source 1..25: mol_type = other DNA
  misc_difference 3: note = R is A or G
  misc_difference 5: note = R is A or G
  misc_difference 9: note = R is A or G
  misc_difference 12: note = R is A or G
  misc_difference 21: note = R is A or G
ttrartttratrctcaccctratca

SEQ ID NO: 5 - DNA, 33 bp, synthetic construct
  source 1..33: mol_type = other DNA
tcgtcggcagcgtcagatgtgtataagagacag

SEQ ID NO: 6 - DNA, 34 bp, synthetic construct
  source 1..34: mol_type = other DNA
gtctcgtgggctcggagatgtgtataagagacag

(19) United States
(12) Patent Application Publication
(10) Pub. No.: US 2026/0085355 A1
(43) Pub. Date: Mar. 26, 2026

(54) METHOD FOR IDENTIFYING DEMENTIA WITH LEWY BODIES IN A SUBJECT

(71) Applicant: ADMIT THERAPEUTICS SL, Esplugues Del Llobregat (ES)

(72) Inventors: Marta BARRACHINA CASTILLO, Esplugues Del Llobregat (ES); Jose Luis MOSQUERA MAYO, Esplugues Del Llobregat (ES); Marta BLANCH LOZANO, Esplugues Del Llobregat (ES)

(21) Appl. No.: 18/880,055

(22) PCT Filed: Jul. 4, 2023
(86) PCT No.: PCT/EP2023/068465

§ 371 Date: Dec. 30, 2024

(30) Foreign Application Priority Data
Jul. 4, 2022 (EP) 22382637.1

(51) Int. Cl. C12Q 1/6883; G16H 10/60; G16H 50/20; G16H 50/70

(52) U.S. Cl. CPC C12Q 1/6883; G16H 10/60; G16H 50/20; G16H 50/70; C12Q 2600/112; C12Q 2600/154

(57) ABSTRACT

It is provided a method for identifying dementia with Lewy bodies (DLB) in a subject, comprising determining in a sample of the subject comprising mitochondrial DNA, the methylation pattern in the D-loop region and/or ND1 gene of the mitochondrial DNA. Further, a classification model, oligonucleotides and kits to perform the method are also provided.

### CROSS-REFERENCE TO RELATED APPLICATIONS

The present application is a national phase filing under 35 U.S.C. § 371 of International Application No. PCT/EP2023/068465, filed on 4 Jul. 2023, which claims the benefit of European Patent Application No. 22382637.1, filed on 4 Jul. 2022, both of which are incorporated herein by reference in their entireties.

### INCORPORATION BY REFERENCE

The sequence listing named “121615_00062_Sequence_Listing_DLB_ST26.xml”, created on Oct. 20, 2025 is 11,667 bytes in size is herein incorporated by reference.

### FIELD OF THE INVENTION

The present invention relates to the fields of medicine and diagnostic or identification of neurodegenerative diseases in a subject and particularly, to methods for the diagnosis and identification of dementia with Lewy bodies in a subject.

### BACKGROUND ART

Dementia with Lewy bodies (DLB), namely Lewy Body Dementia, is the second most common cause of degenerative dementia after Alzheimer's disease (AD) and is characterized by non-motor cognitive alterations preceding parkinsonism. DLB belongs to the heterogeneous group of disorders known as Lewy body diseases (LBDs), which are synucleinopathies with a heterogeneous clinical manifestation. LBDs are characterized by the abnormal accumulation and deposition of misfolded and aggregated alpha-synuclein (a-syn) giving rise to Lewy bodies and Lewy neurites.

DLB shares features with both Alzheimer's and Parkinson's disease (PD). In comparison to AD, people that suffer from DLB are at higher risk of falling, have lower quality of life, greater caregiver burden and a higher mortality rate, beyond other differences in clinical features. On the other hand, DLB and PD with dementia (PDD) present overlapping clinical manifestations, including attention deficits, visual hallucinations, parkinsonism, fluctuating cognitive impairment, and symptoms of rapid eye movement (REM) sleep behavior disorder. However, DLB patients often suffer from dementia before the manifestation of the first parkinsonism symptoms, whereas PD patients are usually the contrary.

In those lines, a new challenge arises regarding the accurate identification and diagnosis of degenerative dementias due to their shared characteristics and common clinical manifestations. Currently, DLB is diagnosed using a combination of medical and neuropsychological tests (e.g., Mini-Mental State, 18F-fluorodeoxyglucose PET, etc.). However, these diagnostic methods sometimes are insufficient to attain a solid and sharp diagnosis, and may lead to an incorrect diagnosis, which can be very dangerous for DLB patients. Patients suffering from DLB are highly sensitive to typical neuroleptics, used to treat delusions and hallucinations. Thus, when DLB patients are falsely diagnosed with AD and consequently treated with typical neuroleptics, they can suffer from significant side effects, which include high levels of mental confusion, aggravation of parkinsonism, extreme drowsiness and, in most severe cases, neuroleptic malignant syndrome. Therefore, it is crucial to develop efficient yet feasible and noninvasive diagnostic tools to improve. DLB diagnosis and, ultimately reach a clear and certain diagnosis of DLB.

Epigenetic mechanisms, such as DNA methylation, modulate the brain transcriptome and have key roles in neurodegeneration. In those lines, genetic and epigenetic variation in the Apolipoprotein E and a-synuclein genes are observed in DLB, and methylation changes in the promoter and intron 1 of SNCA gene, encoding a-synuclein, have also been observed in brain and blood samples from PD and DLB cases (De Boni et al., 2011; Desplats et al., 2011; Funahashi et al., 2017).

Further, Urbizu et al. (2020) performed a detailed review of epigenetic modifications identified for DLB in human cells, post-mortem, and peripheral tissues, which emphasizes that the number of studies focusing on DLB is practically inexistent. Additionally, Chouliaras et al. (2020) summarizes different methylation studies in DLB which identify differentially methylated regions involving both APOE and SNCA genes, along with other new potential targets.

In a wide epigenome study, which analyses several tissues and conditions, Fernandez et al. (2012) identified a group of genes that could serve to distinguish DLB samples from controls in cortical samples. Moreover, Sanchez-Mut et al. (2016) developed a cohort of post-mortem brain samples using whole genome bisulfite sequencing to identify a set of genes that were differentially methylated in DLB and other neurodegenerative conditions. They were able to validate these findings using bisulfite pyrosequencing in a larger cohort which resulted in 1428 differentially methylated regions shared by both PD and DLB subjects when compared to controls.

Contrarily, a recent study compared the blood methylome of DLB and Parkinson's disease with dementia (PDD) patients and found significant differences in blood methylation suggesting that clinical variation in DLB may be reflected in blood epigenome. LASSO method of regularized regression was used with 26 significant differentially methylated positions and a discriminant analysis was used to identify the best predictors to differentiate DLB and PDD cases (Nasamran et al., 2021).

In conclusion, little is known about the role and characteristics of epigenetics in DLB. As discussed above, all studies published to date have focused on epigenetics of the nuclear genome and remarkably, no unambiguous outcome can be derived from such studies, as the significance and potential similarities or differences in the methylation pattern of DLB and other neurodegenerative diseases is yet unclear.

On the other hand, WO2015/144964 A2 discloses potential mitochondrial methylation patterns for both AD and PD, which result from the analysis of brain samples from subjects after death, said results corresponding to very early stages ofresearch. Further, data disclosed in WO2015/144964 A2 is also discussed in Blanch et al. (2016). Similarly, Stoccoro et al. (2017 & 2022) present mitochondrial methylation patterns of late-onset AD patients compared to controls, and later compare said patterns between subjects diagnosed with mild cognitive impairment, AD patients and controls.

At present, there is no cure nor treatment capable of decelerating the progression of DLB, and only some drugs are available for symptomatic treatment (e.g., cholinesterase inhibitors). Therefore, there is a strong need to perform clinical trials, which require an accurate recruitment to succeed, and ultimately lead to the discovery of new treatment options for DLB patients. Overall, a precise, reliable, non-invasive and feasible method for diagnosing and identifying DLB is highly desirable to improve the selection of the most adequate option of treatment for a patient, as well as to improve recruitment efficiency of clinical trials and quicken therapeutic development of DLB.

### SUMMARY OF THE INVENTION

One problem to be solved by the present invention is to provide a method for diagnosing or identifying Dementia with Lewy bodies (herein referred to as DLB) in a subject.

The present invention discloses a method capable of identifying samples from subjects suffering from DLB, and consequently diagnose DLB in a subject in need. Said distinction which allows for the diagnosis of DLB is obtained through the processing of feasible and accessible samples such as blood samples. Further, the present invention also discloses a method capable of calculating or determining a score to identify the presence of/diagnose DLB in a subject, and consequently classify subjects according to said diagnosis. The method disclosed herein is further capable to identify subjects which may not have yet advanced to DLB, but that are at high risk of developing DLB. This method comprises the execution of a classification model capable of processing more than one dataset which include biomarker screening data (i.e., mitochondrial methylation data) and other relevant clinical data (e.g., MMSE). Said biomarker screening data is obtained from blood samples, therefore allowing for a fast, non-invasive, and effective methodology for the diagnosis/identification of such disease.

The use of mitochondrial markers to diagnose other neurodegenerative diseases, such as AD and PD, had been disclosed in WO2015/144964 A2. However, no information on the potential role nor significance of mitochondrial markers in DLB has been previously described, neither has its potential usefulness in the diagnosis or identification of such disease.

In terms of other epigenetic markers for DLB, very few studies have been published on this regard, all of which focused on the analysis of epigenetics of the nuclear genome. However, no clear conclusion can be drawn from such studies. While some have identified differences in the methylation of certain positions/regions in samples from DLB subjects compared to subjects suffering from other neurodegenerative diseases and control subjects, other studies suggest a shared pattern of methylation of the nuclear genome for DLB and other neurodegenerative diseases (e.g., PD). Altogether, no hint on the characteristics or significance of potential patterns of mitochondrial DNA (herein referred to as mtDNA) methylation in DLB has been disclosed so far.

Surprisingly, the inventors have found that samples from DLB patients indeed show a distinguishing pattern of mtDNA methylation in certain regions of mtDNA, e.g., D-loop region, whereas other regions studied herein do not contribute to the recognition of DLB patients when compared to controls. As shown in the present invention, the difference in mitochondrial methylation of sites included in the D-loop region and in the ND1 gene, comprising all three possible contexts (i.e., CpG, CHG and CHH sited), is highly statistically significant for DLB subjects.

As shown herein, several methylation sites which show highly significant methylation patterns correspond to CHG sites in the D-loop region. These results are remarkable considering the common focus which epigenetics studies have on CpG sites methylation. Methylation of non-CpG sites is usually considered as not or less relevant than methylation of CpG sites, which potentially leads to biased results. In order to avoid said bias, inventors of the present invention have used a set of primers which equally consider the potential methylation of all sites. Further, the primers used herein are exceptionally efficient in the detection of methylation in mtDNA extracted from blood samples (see EXAMPLE 1). Finally, examples of the present invention gather information from blood samples, which are an example of non-invasive and feasible samples, which do not require expensive processing.

Working examples herein provide detailed experimental data demonstrating an efficient processing of blood samples for the detection and calculation of mtDNA methylation. As a result, an efficient diagnosis of DLB in subjects in need is feasible. Furthermore, the information regarding said mitochondrial methylation is combined with other relevant clinical data, and altogether processed by a classification model. As a result, the method provided herein, determines a score corresponding to presence (i.e., diagnosis) of DLB in a subject.

EXAMPLE 1 shows the method for detecting mtDNA methylation which comprises collecting blood samples, extracting and treating DNA (bisulfite treatment), and preparing the amplicon library to detect, quantify and normalize methylation in mtDNA sites of interest. The use of degenerated primers resulted in an extraordinarily high sensitivity in the detection of mtDNA methylation in all three contexts (i.e., CpG, CHG, CHH). Further, the comparison of methylation levels between DLB subjects and controls in all different contexts of the D-loop region resulted in a high number of significant differentially methylated sites.

EXAMPLE 2 shows the development of a classification model which considers not only data on the methylation sites of interest, but also other relevant clinical data (e.g., MMSE). Several supervised methods have been applied in order to select the most appropriate model (i.e., the random forest). Basically, after pre-processing the data to handle appropriate levels of categorical variables, missing values, outliers and normalize the continuous variables, the dataset is split into training and validation sets. Each method builds the classification model differently. In the case of the random forest an ensemble of decision trees, is constructed using a random subset of the training data, with each trees using a random subset of features (i.e., clinical and or methylation variables) at each split. The decision trees are grown to their maximum depth or stopping criterion is met. To classify a new observation, it is pass through each tree in the random forest, and the final predicted label is determined based on the majority vote from all trees. The performance of the model is evaluated using metrics such as Accuracy and Kappa, which measures the proportion of correctly classified individuals and the agreement between predicted and true labels respectively. After building the random forest model, the test data is classified by assigning labels (i.e., either DLB or Control) based on the highest probability predicted by the model. Performance statistics such as Accuracy, Kappa, sensitivity, specificity, precision, recall, and FI-score are computed to evaluate the classification results. These measures provide an assessment of the accuracy and agreement of the model, and its ability to correctly identify positive and negative instances.

EXAMPLE 3 shows the methylation patterns of a higher number of samples (84). The comparison of methylation levels between DLB subjects and controls in all different contexts of the D-loop region and of the ND1 gene resulted in a high number of significant differentially methylated sites. EXAMPLE 1 also shows the development of a classification model which considers data on the methylation sites provided herein. In this example during the preprocessing step, in order to reduce the dimensionality of the variables (i.e., methylation measures), two methods were applied, first an agglomeration feature selection based on the Spearman Rank Correlation and later a Principal Component Analysis (PCA). Based on the top 10 components resulting from the PCA, the model was built following the same approach as the described in Example 2. That is, several supervised methods were performed in order to select the most appropriate model (i.e., random forest). The resulting trained model showed an outstanding high performance suggested by an overall accuracy score of 0.83 and a Kappa value of 0.65. Therefore, this classification model is able to identify dementia with Lewy Body in a subject by rapidly processing their individual information with a remarkably good performance.

Further, it is worth noting that this first prototype uses only methylation data, and that the methylation pattern provided herein is robust enough to discriminate between DLB patients and healthy controls. When a larger number of samples can be acquired and different neuropsychological variables can be introduced into the algorithm; an even more efficient classification model is expected to become available.

In this regard, it is noted that the acquisition of samples and clinical information of subjects is a complicated process for many reasons: suitable subjects are limited, and they must have been monitored for long periods of time; the accessibility to samples is limited; the quality of the samples can be compromised; the information on subjects include missing data or are heterogenous due to e.g., the variability between neuropsychological tests used in each country, etc. Overall, the data available for the development of a classification model is not to be taken for granted as it is the result of a burdensome and difficult process.

Accordingly, a first aspect of the invention relates to a method for identifying dementia with Lewy bodies in a subject, comprising: (a) determining in a sample of the subject comprising mitochondrial DNA, the methylation pattern in the D-loop region and/or the ND1 gene of the mitochondrial DNA, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6.

A second aspect relates to a method for identifying dementia with Lewy bodies in a subject, comprising:
(a) determining in a sample of the subject comprising mitochondrial DNA, the methylation pattern in the D-loop region and/or the ND1 gene of the mitochondrial DNA, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6; and
(b) combining the methylation pattern of one or more sites determined in step (a), optionally with at least one clinical variable of the subject, wherein said combining is performed using a classification model for determining a score which correlates to the identification of dementia with Lewy Body in the subject.

Other aspects of the invention relate to a method for diagnosing DLB in a subject; a method for identifying a subject with DLB; a method for identifying a subject suitable for treatment of DLB; a method for selecting a subject for submission to treatment for DLB; a method for selecting a suitable therapy to treat a subject with DLB; a method for classifying a subject according to the presence or absence of DLB; a method for treating DLB in a subject comprising administering a treatment for DLB if the subject has been identified with DLB; a method for treating DLB comprising determining the presence or absence of said dementia; and a method for treating DLB in a subject comprising assigning, prior to the administration, a dementia classification state to the subject, using the methods described above. Other aspects of the invention relate to methods for monitoring the progression of DLB in a subject and methods for monitoring and treating a subject suffering from DLB, using the methods described above.

Other aspects of the invention relate to oligonucleotides and kits comprising oligonucleotides for use in the determination of a methylation pattern of mitochondrial DNA to identify DLB in a subject, wherein the methylation pattern is determined in at least one methylation site selected from the group consisting of:
(i) the CpG sites in the D-loop region shown in Table 1,
(ii) the CHG sites in the D-loop region shown in Table 3, and
(iii) the CHH sites in the D-loop region shown in Table 5
(iv) the CpG sites of the ND1 gene shown in Table 2,
(v) the CHG sites of the ND1 gene shown in Table 4, and
(vi) the CHH sites of the ND1 gene shown in Table 6.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### DESCRIPTION OF THE DRAWINGS

FIG. 1 shows the boxplots of methylation percentages for each position for controls subjects (n=18) and DLB subjects (n=18) in ND1 gene for CpG context.

FIG. 2 shows the median and confidence interval (95%) of methylation percentages for each individual including control subjects (n=18) and DLB subjects (n=18) in ND1 gene for CpG context.

FIG. 3 shows the boxplots of methylation percentages for each position for controls subjects (n=18) and DLB subjects (n=18) in ND1 gene for CHG context.

FIG. 4 shows the median and confidence interval (95%) of methylation percentages for each individual including control subjects (n=18) and DLB subjects (n=18) in ND1 gene for CHG context.

FIG. 5A-5D show the boxplots of methylation percentages for each position for controls subjects (n=18) and DLB subjects (n=18) in ND1 gene for CHH context.

FIG. 6 shows the median and confidence interval (95%) of methylation percentages for each individual including control subjects (n=18) and DLB subjects (n=18) in ND1 gene for CHH context.

FIG. 7 shows the boxplots of methylation percentages for each position for controls subjects (n=18) and DLB subjects (n=18) in D-loop region for CpG context.

FIG. 8. shows the median and confidence interval (95%) of methylation percentages for each individual including control subjects (n=18) and DLB subjects (n=18) in D-loop region for CpG context.

FIG. 9 shows the boxplots of methylation percentages for each position for controls subjects (n=18) and DLB subjects (n=18) in D-loop region for CHG context.

FIG. 10 shows the median and confidence interval (95%) of methylation percentages for each individual including control subjects (n=18) and DLB subjects (n=18) in D-loop region for CHG context.

FIG. 11A-B show the boxplots of methylation percentages for each position for controls subjects (n=18) and DLB subjects (n=18) in D-loop region for CHH context.

FIG. 12 shows the median and confidence interval (95%) representation of methylation percentages for each individual including control subjects (n=18) and DLB subjects (n=18) in D-loop region for CHH context.

FIG. 13 shows the boxplots of methylation percentages for each position for controls subjects (n=42) and DLB subjects (n=42) in ND1 gene for CpG context.

FIG. 14 shows the boxplots of methylation percentages for each position for controls subjects (n=42) and DLB subjects (n=42) in ND1 gene for CHG context.

FIG. 15 shows the boxplots of methylation percentages for each position for controls subjects (n=42) and DLB subjects (n=42) in ND1 gene for CHH context.

FIG. 16 shows the boxplots of methylation percentages for each position for controls subjects (n=42) and DLB subjects (n=42) in Dloop region for CpG context.

FIG. 17 shows the boxplots of methylation percentages for each position for controls subjects (n=42) and DLB subjects (n=42) in Dloop region for CHG context.

FIG. 18 shows the boxplots of methylation percentages for each position for controls subjects (n=42) and DLB subjects (n=42) in Dloop region for CHH context.

FIG. 19 shows Accuracy and Kappa metrics of the supervised learning models that were trained in EXAMPLE 3.

FIG. 20 displays the ROC curve showing the performance of the classification model for the Dementia with Lewy Bodies patients (DLB) vs Controls (CTL) at all classification thresholds using the model built with the Random Forest method of EXAMPLE 3.

### DETAILED DESCRIPTION OF THE INVENTION

For the avoidance of doubt, the methods provided herein do not involve diagnosis practiced on the human or animal body. The methods of the invention are particularly conducted on a sample that has previously been removed from the subject. The kits provided herein can include means for extracting the sample from the subject.

### Definitions

Diagnosis: The term “diagnosis” refers to both the process of trying to determine and/or identify a possible disease in a subject, that is to say the diagnostic procedure, as well as the opinion reached through this process, i.e., the diagnostic opinion. As such, it can also be seen as an attempt to classify the status of an individual in separate and distinct categories that allow medical decisions about treatment and prognosis to be taken. As will be understood by the person skilled in the art, such diagnosis may not be correct for 100% of the subjects to be diagnosed with, although it is preferred that it is. However, the term requires that a statistically significant portion of subjects can be identified as suffering from DLB in the context of the invention, or a predisposition thereto. The person skilled in the art may determine whether a part is statistically significant using different well known statistical evaluation tools, for example, by determining confidence intervals, determining the value of p, an adjusted value of p, Student's t-test, the Mann-Whitney test, etc. Particular confidence intervals are at least 50%, at least 60%, at least 70%, at least 80%, at least 90% or at least 95%. P values or adjusted P values are particularly 0.1, 0.05, 0.025, 0.001 or lower.

Dementia with Lewy bodies: The term “Dementia with Lewy bodies” or “Lewy Body Dementia” or DLB refers to a degenerative dementia characterized by a slow and progressive cognitive decline. Other characteristic features of DLB are spontaneous parkinsonism, recurrent visual hallucinations, fluctuating cognition, rapid eye movement (REM) sleep behaviour disorder (RBD) and severe sensitivity to antipsychotic medications with respect to developing extrapyramidal symptoms. DLB does not comprise standardized diseases stages, as other neurodegenerative diseases, because it is a highly variable and fluctuant disease, wherein the progression of the disease can differ considerably between patients. Symptoms do not only vary greatly between patients, but the symptomatology of a patient can also fluctuate a lot in short periods of time.

The primary symptom of DLB is dementia, usually accompanied by impairment in visual and spatial perceptions (i.e., difficulties in the correct perception of distance and depth and misidentification of objects), along with further difficulties in cognitive functions such as planning, multitasking, problem solving, and reasoning. Dementia can also include changes in mood and behavior, poor judgment, loss of initiative, mental confusion in terms of time and place, and difficulty with language and numbers. Further, DLB symptoms also include cognitive fluctuations and hallucinations, and memory loss may arise in later stages of the disease. Cognitive fluctuations are common and include unpredictable changes in concentration, attention, alertness, and wakefulness which can vary greatly from one day to another, or even throughout the same day. Hallucinations can be visual and/or auditory, however visual hallucinations are more common, affecting up to 80% of DLB patients and are generally very realistic and detailed. Hallucinations are perceptions in the absence of an external stimulus that has the qualities of a real perception.

Regarding motor symptoms, parkinsonism is a common symptom in DLB patients, which can affect each patient very differently. Parkinsonism in DLB causes bradykinesia, difficulty in walking, rigidity, and postural instability. DLB patients can also present REM sleep behavior disorder, which is a parasomnia, with abnormal dream-enacting behaviour during REM sleep. It can include vivid dreaming, talking in one's sleep and violent movements. Further, some DLB patients suffer from dysautonomia or autonomic dysfunction, resulting from impaired functioning of the autonomic nervous system (ANS).

Subject: The terms “subject”, “patient”, “individual”, and variants thereof are used interchangeably herein and refer to any mammalian subject, particularly a human subject. The term does not denote a particular age or sex:

Sample comprising mitochondrial DNA: The expression “sample comprising mitochondrial DNA” as used herein refers to any sample that can be obtained from a subject in which there is genetic material from the mitochondria suitable for detecting the methylation pattern.

Mitochondrial DNA: The term “mitochondrial DNA” or “mtDNA” as used herein, refers to the genetic material located in the mitochondria of living organisms. It is a closed, circular double-stranded molecule. In humans it consists of 16,569 base pairs, containing a small number of genes, distributed between the H chain and L chain. Mitochondrial DNA encodes 37 genes: two ribosomal RNA, 22 transfer RNA and 13 proteins that participate in oxidative phosphorylation.

Methylation pattern or methylation status: The term “methylation pattern” as used herein refers but is not limited to the presence or absence of methylation of one or more nucleotides, particularly the methylation in cytosines. Thus, said one or more nucleotides are comprised in a single nucleic acid molecule. Said one or more nucleotides are capable of being methylated or not. The term “methylation status” can also be used when only considering a single nucleotide. A methylation pattern can be quantified; in the case it is considered more than one nucleic acid molecule.

D-loop region: This term, as used herein, refers to a region of non-coding mtDNA, which acts as a promoter for both the heavy and the light strains of the mtDNA, and contains essential transcription and replication elements. The D-loop region contains approximately 1120 base pairs, visible under electron microscopy, which is generated during H chain replication for the synthesis of a short segment of the heavy strand, 7S DNA. The human D-loop region sequence is deposited in the GenBank database under the accession number NC_012920.1.

ND1 gene; The term “ND1 gene” or “NADH dehydrogenase 1” or “ND1 mt”, as used herein, refers to the gene localized in the mitochondrial genome that encodes the protein NADH dehydrogenase 1 or ND1. The human ND1 gene sequence is deposited in the GenBank database under the accession number NC_012920.1. The ND1 protein is part of the enzyme complex called complex I which is active in the mitochondria and is involved in the process of oxidative phosphorylation. In some embodiments, the term “ND1 gene” can refer to the gene above further comprising comprise approximately 50 additional base pairs in one or the two extremes of the sequence.

CpG site: This term, as used herein, to distinguish this single-stranded linear sequence from the CG base-pairing of cytosine and guanine for double-stranded sequences. “CpG” is an abbreviation for “C-phosphate-G”, i.e., cytosine and guanine separated by only a phosphate; phosphate binds together any two nucleosides in the DNA. The term “CpG” is used to distinguish this linear sequence of CG bases pairing of guanine and cytosine. Cytosine in the CpG dinucleotides can be methylated to form 5-methylcytosine.

CHG site: This term, as used herein, refers to DNA regions, particularly mitochondrial DNA regions, where a cytosine nucleotide and a guanine nucleotide are separated by a variable nucleotide (H) which can be adenine, cytosine, or thymine. Cytosines of the CHG site can be methylated to form 5-methylcytosine.

CHH site: This term, as used herein, refers to DNA regions, particularly regions of mitochondrial DNA, where a cytosine nucleotide is followed by a first and a second variable nucleotide (H) which can be adenine, cytosine, or thymine. Cytosines of the CHH can be methylated to form 5-methyl cytosine.

Determination of the methylation pattern in a CpG site: The term “determination of the methylation pattern in a CpG site” as used herein, refers to the determination of the methylation status of a particular CpG site. The determination of the methylation pattern of a CpG site can be performed by multiple processes known to the person skilled in the art.

Determination of the methylation pattern in a CHG site: This term, as used herein, refers to the determination of the methylation status of a particular CHG site. The determination of the methylation pattern of a CHG site can be performed by multiple processes known to the person skilled in the art.

Determination of a methylation pattern in a CHH site: This term, as used herein, refers to determining the methylation status of a particular CHH site. The determination of the methylation pattern of a CHH site you can be performed by multiple processes known to the person skilled in the art.

To determine the methylation pattern in mitochondrial DNA, samples can be chemically treated so that all cytosine unmethylated bases are modified at uracil bases, or another base which differs from cytosine in terms of base pairing behavior, while the bases of 5-methylcytosine remain unchanged. The term “modify” as used herein means the conversion of an unmethylated cytosine to another nucleotide that will distinguish the unmethylated cytosine from the methylated cytosine. The conversion of unmethylated cytosine bases, but not methylated, in the sample containing mitochondrial DNA is carried out with a conversion agent. The term “conversion agent” or “conversion reagent” as used herein, refers to a reagent capable of converting an unmethylated cytosine to uracil or another base that is differentially detectable to cytosine in terms of hybridization properties. The conversion agent is particularly a bisulfate such as bisulfites or hydrogen sulfite. However, other agents that similarly modify unmethylated cytosine, but not methylated cytosine can also be used in this method of the invention, such as hydrogen sulfite. The reaction is performed according to standard procedures. It is also possible to carry out the conversion enzymatically, e.g., using cytidine deaminases specific methylation.

Reference sample: This term, as used herein, refers to a sample containing mitochondrial DNA obtained from a subject not suffering from DLB. Thus, the DNA sequence of reference is NCBI Reference Sequence: NC_012920.1. In particular, said term refers to a small number of 5-methylcytosines in one or more CpG sites in the D-loop region shown in Table 1, in one or more CpG sites of the ND1 gene shown in Table 2, in one or more sites CHG sites in the D-loop region shown in Table 3, in one or more CHG sites in the ND1 gene shown in Table 4, one or more CHH sites in the D-loop region shown in Table 5 and/or one or more CHH sites in the ND1 gene shown in Table 6 in a sequence of mitochondrial DNA as compared to the relative amount of 5-methylcytosines present in said one or more CpG sites, one or more CHG sites and/or one or more CHH sites in a subject sample.

Treatment of dementia with Lewy bodies: This term, as used herein, refers to treatment for the disease or any related symptoms. Such treatments can include medications, epigenetic treatments, or any cognitive stimulating treatment. Some cognitive stimulating treatments are digital cognitive treatments (i.e., using digital devices). This term can include any treatment known in the art for DLB or future developments. Treatments of DLB can include but are not limited to drugs directed to the treatment of symptoms of the dementia, which can include cholinesterase inhibitors (e.g., rivastigmine), drugs directed to the treatment of parkinsonism (e.g., levodopa), or drugs directed to the treatment of psychotic symptoms such as hallucinations, which can be treated with atypical antipsychotics (e.g., quetiapine). Treatments of DLB can include preventive treatments for subjects that have been identified of being at high risk of developing DLB but are not yet in a dementia stage. Preventive treatments include any method known in the art of cognitive stimulation. Preventive treatments can include suitable future developments.

Dementia Stage Classification (DSC): The term “Dementia Stage Classification”, as used herein, refers to the classification of subjects based on diagnosis or identification of a dementia. This classification results from the score determined by the classification model disclosed herein, which determines the presence or absence of a dementia. The classification model according to examples of the present invention classifies subjects with DLB and subjects without. Subjects without DLB, are not necessarily healthy subjects, but maybe suffering from other diseases such as Alzheimer's disease. Therefore, “Dementia Stage Classification” includes two classes: subjects with dementia with Lewy bodies and subjects without DLB.

Specifically hybridizes: The expression “specifically hybridizes” or “capable of hybridizing in a specific form”, as used herein, refers to the ability of an oligonucleotide or of a polynucleotide of specifically recognizing a specific sequence of interest, e.g., D-loop region or ND1 gene. The sequence of interest may refer to the reference sequence or the sequence resulting from certain modification treatment, e.g., bisulfite treatment wherein unmethylated cytosines are modified to uracil. As used herein the term “hybridization”, refers the process of combining two nucleic acid molecules or single-stranded molecules with a high degree of similarity resulting in a simple double-stranded molecule by specific pairing between complementary bases. Normally, hybridization occurs under very stringent conditions or moderately stringent conditions.

Oligonucleotide: The term “oligonucleotide” as used herein, is used indistinctly with “primer” and “nucleic acid sequence” and refers to a DNA molecule or short RNA, with up to bases in length. Oligonucleotides of the invention are particularly DNA molecules at least 2, at least 3, at least 4, at least 5, at least 6, at least 7, at least 8, at least 9, at least 10, at least 15, at least 20, at least 30, at least 40, at least 45 or 50 bases of length.

Score: This term, as used herein, refers to one or more values, particularly a single value that can be used as a component in a classification model for the diagnosis or identification of a disease in a subject. Such single value can be calculated or determined (i.e., estimated) by combining the values of descriptive features processed by an interpretation function or algorithm. Scores can be scores of 0.0-1, with scores over 0.5 indicating the presence of a disease in a subject and 0 indicating the absence of said disease. Scores can be classified in groups, i.e., classes, such as non, low, intermediate, and high.

Computer-implemented methods: The term “computer-implemented method” refers to methods in which all or some steps of a method are carried out by a computer, another programmable apparatus, or a network of computers.

Supervised machine learning methods: This term refers to methods which use a training set to generate a desired output to develop a model. This training dataset includes inputs and correct outputs, which allow the model to learn over time. Its accuracy is measured through at loss function, and its model can be adjusted until a generalization error has been sufficiently minimized.

Non-supervised machine learning methods: The term “non-supervised learning methods”, also referred as “unsupervised learning methods” refer to methods which use machine learning algorithms to analyze unlabeled dataset. These methods recognize similarities and differences in information to detect data groups or patterns. These methods are commonly used for clustering, association, and dimensionality reduction of datasets.

Deep learning methods: This term refers to a sub field of machine learning that uses neural networks models with multiple layers to automatically extract and learn features and patterns from raw data. A layer in a neural network is a set of interconnected artificial neurons (aka nodes). These neurons receive input data and apply specific types of computational tasks to it. Usually, these tasks involve weighted sums and mathematical activation functions. The output of a layer is passed to the next layer and so on. This process allows the network to gradually learn more complex features, attributes, and patterns in the data. There are different types of layers in a neural network. Some examples are fully connected layers, convolutional layers, pooling layers, and recurrent layers.”

Artificial intelligence methods: This term refers to methods using artificial intelligence, defined as the capacity of a computer or a computer-controlled robot to emulate human's capabilities to respond to certain stimuli.

Classification model: The term “classification model” is herein indistinctly referred to as “classifying model”. The classification model can be developed using e.g., a type of supervised learning method capable of accurately assigning test data/new observations into specific categories based on training data. The model is trained to: learn from the given training dataset and is consequently capable to classify new datasets into certain score/number or class/group. Examples of classification algorithms are Logistic Regression, Linear Discriminant Analysis (LDA), Classification and Regression Trees (CART), k-Nearest Neighbors (kNN), Naive Bayes (NB), Support Vector Machines (SVM) with a linear kernel, Random Forest (RF), Neural Network (NNET), Generalized Boosted, Regression Model (GBM) and Binominal Logistic Regression (GLM).

Converting: This term, as used herein means subjecting the one or more descriptive features to an interpretation function or algorithm for a predictive model of disease, particularly DLB. In some embodiments, the interpretation function can also be produced by a plurality of predictive models. In one embodiment, the predictive model includes a regression model and a Bayesian classifier or score. In one embodiment, an interpretation function comprises one or more terms associated with one or more biomarkers or sets of biomarkers. In one embodiment, an interpretation function comprises one or more terms associated with the presence or absence or spatial distribution of the specific cell types disclosed herein. In one embodiment, an interpretation function comprises one or more terms associated with the presence, absence, quantity, intensity, or spatial distribution of the morphological features of a cell in a cell sample. In one embodiment, an interpretation function comprises one or more terms associated with the presence, absence, quantity, intensity, or spatial distribution of descriptive features of a cell in a cell sample.

### Methods of Identifying DLB

One aspect of the invention relates to a method for identifying DLB in a subject, comprising determining in a sample from said subject comprising mitochondrial DNA, the methylation pattern in the D-loop region and/or the ND1 gene, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6.

A computer-implemented method for identifying DLB in a subject can comprise receiving a methylation pattern of the D-loop region and/or of a ND1 gene of the mitochondrial DNA from a sample from the subject, and a classification model assigning a score to the subject.

In an embodiment, the method comprises:
(a) determining in a sample of the subject comprising mitochondrial DNA, the methylation pattern in the D-loop region, and/or the ND1 gene of the mitochondrial DNA, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2, and
(v) the CHG sites of the ND1 gene shown in Table 4,
and wherein the methylation pattern is determined in at least one of (vi) the CHH sites of the ND1 gene shown in Table 6.

Alternatively, the method of the invention can be formulated as a method for diagnosing DLB in a subject, comprising the steps as described above. Alternatively, the method of the invention can be formulated as a method for identifying a subject with DLB, comprising the steps as described above.

In some embodiments, the methods, particularly a computer-implemented method, comprise determining the methylation pattern in the D-loop region, wherein hypomethylation in at least one site of said CpG sites in the D-loop region, wherein hypomethylation in at least one site of said CHG sites in the D-loop region and/or wherein hypomethylation in at least one site of said CHH sites in the D-loop region is indicative that the subject suffers from DLB. Alternatively, these methylation patterns are indicative that the subject is at elevated risk of developing DLB.

In an embodiment, the methods further comprise determining the methylation pattern in the ND1 gene, wherein the methylation pattern is determined in at least one site selected from the group consisting of:
(iv) the CpG sites of the ND1 gene shown in Table 2,
(v) the CHG sites of the ND1 gene shown in Table 4 and,
(vi) the CHH sites of the ND1 gene shown in Table 6.

In some embodiments, the methods comprise determining the methylation pattern in the ND1 gene, wherein no significant differences on methylation levels in at least one site of said CpG sites in the ND1 gene, wherein no significant differences on methylation levels in at least one site of said CHG sites in the ND1 gene and/or wherein no significant differences on methylation levels in at least one site of said CHH sites in the ND1 gene is indicative that the subject suffers from DLB. Alternatively, these methylation patterns are indicative that the subject is at elevated risk of developing DLB.

The statistical significance of methylation patterns in each methylation site can vary according to the threshold of significance set, i.e., adjusted p-value. In some embodiments, p-values are adjusted using False Discovery Rate (FDR) methods of Family Wise Error Rate (FWER) methods. In a particular embodiment, p-values are adjusted using FDR methods, Differences in methylation patterns can be regarded as statistically significant or not depending on the threshold of significance stablished, which may be, for example, at adjusted p-value ≥0.05 or adjusted p-value ≥0.1. In some embodiments, differences in methylation patterns are not regarded as statistically significant if adjusted p-value ≥0.05. In other embodiments, differences in methylation patterns are not regarded as statistically significant if adjusted p-value ≥0.1. In another embodiment, the methods further comprise determining the methylation pattern in the ND1 gene, wherein no significant differences on methylation levels in at least one site of said CpG sites in the ND1 gene, wherein no significant differences on methylation levels in at least one site of said CHG sites in the ND1 gene and/or wherein hypomethylation in at least one site of said CHH sites in the ND1 gene is indicative that the subject suffers from DLB. Alternatively, these methylation patterns are indicative that the subject is at elevated risk of developing DLB.

In some embodiments, the methods comprise determining the methylation pattern in the D-loop region
and in the ND1 gene, wherein:
hypomethylation in at least one site of said CpG sites in the D-loop region,
hypomethylation in at least one site of said CHG sites in the D-loop region, and/or
hypomethylation in at least one site of said CHH sites in the D-loop region; and
no significant differences on methylation levels in at least one site of said CpG sites in the ND1 gene,
no significant differences on methylation levels in at least one site of said CHG sites in the ND1 gene,
and/or
no significant differences on methylation levels or hypomethylation in at least one site of said CHH sites in the ND1 gene,
is indicative that the subject suffers from DLB.

The methods described herein are useful for the diagnosis of DLB in a subject and can be used clinically to make treatment decisions by choosing the most appropriate treatment modalities for any particular patient. The methods are also useful for classifying a subject in order e.g., to enter in a clinical trial or to receive adequate treatment in an early stage. Thus, the application of the methods disclosed herein can improve clinical outcomes by matching patients to therapies and also improve the accuracy in the selection of patients required for clinical trials to success in evaluating potential DLB treatments.

In those lines, another aspect relates to a method, particularly a computer-implemented method, for identifying a subject suitable for treatment of DLB, the method comprising determining in a sample from said subject comprising mitochondrial DNA, the methylation pattern in the D-loop region and/or the ND1 gene, wherein the methylation pattern is determined in at least one site selected from the group consisting of
(i) the CpG sites of the D-loop region shown in Table 1,
(ii) the CHG sites in the D-loop region shown in Table 3, and
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6.

Alternatively, this aspect can be formulated as a method for selecting a subject for submission to treatment for DLB, comprising the steps as described above.

In some embodiments, the methods (i.e., for identifying a subject suitable for treatment of DLB or for selecting a subject for submission to treatment for DLB) further comprise administering treatment for DLB to the subject.

In some embodiments, the methods comprise determining the methylation pattern in the D-loop region, wherein hypomethylation in at least one site of said CpG sites in the D-loop region, wherein hypomethylation in at least one site of said CHG sites in the D-loop region and/or wherein hypomethylation in at least one site of said CHH sites in the D-loop region is indicative that the subject suffers from DLB or that the subject is at elevated risk of developing DLB.

In some embodiments, the methods further comprise determining the methylation pattern in the ND1 gene, as described above.

The diagnosis or identification of DLB in a subject, indicates that a treatment for DLB can be administered to the subject. If the subject is not diagnosed or identified with DLB, the subject cannot be subjected to any treatment for DLB, and can be suspected to suffer from another disease, and thus undergo clinical follow-up and/or other diagnostic methods. Alternatively, the subject can be treated for other dementias if required.

Therefore, another aspect of the invention is directed to a method, particularly a computer-implemented method, for selecting a suitable therapy to treat a subject with DLB comprising determining in a sample from said subject comprising mitochondrial DNA, the methylation pattern in the D-loop region and/or the ND1 gene, wherein the methylation pattern is determined in at least one site selected from the group consisting of:
(i) the CpG sites of the D-loop region shown in Table 1,
(ii) the CHG sites in the D-loop region shown in Table 3,
(ii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6.

Alternatively, this aspect of the invention can be formulated as a method for classifying a subject according to the presence or absence of DLB, comprising the steps as described above.

In some embodiments, the methods (i.e., for selecting a suitable therapy to treat a subject suffering from DLB and for classifying a subject according to the presence or absence of DLB) further comprise administering a treatment for DLB to the subject, when the subject has been classified as having DLB.

In some embodiments, the methods further comprise determining the methylation pattern in the ND1 gene, as described above.

Another aspect of the invention relates to a method, particularly a computer-implemented method, for treating DLB in a subject, comprising administering a treatment for DLB if the subject has been identified with DLB by determining in a sample from said subject comprising mitochondrial DNA the methylation pattern in the D-loop region and/or the ND1 gene, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6.

Alternatively, this aspect of the invention can be formulated as a method for treating DLB, comprising determining the presence or absence of said dementia, by determining in a sample from said subject comprising mitochondrial DNA, the methylation pattern in the D-loop region and/or the ND1 gene, wherein the methylation pattern is determined in at least one site selected from the group consisting of:
(i) the CpG sites of the D-loop region shown in Table 1,
(ii) the CHG sites in the D-loop region shown in Table 3,
(ii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6, wherein a treatment for DLB is administered if the subject has been identified with DLB.

In some embodiments, the methods comprise determining the methylation pattern in the D-loop region, wherein hypomethylation in at least one site of said CpG sites in the D-loop region, wherein hypomethylation in at least one site of said CHG sites in the D-loop region and/or wherein hypomethylation in at least one site of said CHH sites in the D-loop region is indicative that the subject suffers from DLB.

In some embodiments, the methods further comprise determining the methylation pattern in the ND1 gene, comprising the steps as described above.

Besides, other aspects relate to a method for treating a subject, particularly a subject diagnosed with DLB, administering a treatment for DLB wherein, prior to the administration, the subject is assigned with a dementia state classification (DSC), determined by applying a classification model to a methylation pattern of the D-loop region, and/or of the ND1 gene of the mitochondrial DNA from a sample from the subject, wherein the DSC is selected from subject with DLB and subject without. DLB. In an embodiment, when subject is assigned with DSC presence of DLB, the subject is suitable to be administered with a treatment for DLB. In another embodiment, if the subject is assigned as not suffering from DLB, the subject cannot be subjected to any treatment for DLB but can undergo clinical follow-up or be tested for alternative diseases, such as AD. Alternatively, the subject can be treated for other dementias if required.

This aspect of the invention can alternatively be formulated as a method, particularly a computer-implemented method, for treating a subject comprising:
(i) assigning, prior to the administration, a DCS to the subject by applying a classification model to a methylation pattern of the D-loop region, and/or of the ND1 gene of the mitochondrial DNA from a sample from the subject, wherein the DSC is selected from subject with DLB and subject without DLB; and,
(ii) administering to the subject a treatment for DLB if DSC is subject with DLB or no-treatment for DLB or clinical follow-up or administration of a therapy for other dementias if DSC is subject without DLB.

In some embodiments, the methylation pattern in the D-loop region of the mitochondrial DNA is determined in at least one site selected from the group consisting of:
(i) the CpG sites in the D-loop region shown in Table 1,
(ii) the CHG sites in the D-loop region shown in Table 3, and
(iii) the CHH sites in the D-loop region shown in Table 5.

In other embodiments, the methods further comprise determining the methylation pattern in the ND1 gene of the mitochondrial DNA in at least one site selected from the group consisting of:
(iv) the CpG sites of the ND1 gene shown in Table 2;
(v) the CHG sites in the ND1 gene shown in Table 4, and
(vi) the CHH sites in the ND1 gene shown in Table 6.

In some embodiments, the methods described herein comprise:
a) determining in a sample of the subject comprising mitochondrial DNA, the methylation pattern in the D-loop region, and/or the ND1 gene of the mitochondrial DNA,
wherein the methylation pattern is determined in at least one of the CHH sites of the ND1 gene shown in Table 6, and in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2, and
(v) the CHG sites of the ND1 gene shown in Table 4.

TABLE 1
List of CpG sites (i.e., positions) from
16491 and 202 in the D-loop region.

| | |
|---|---|
| CpG sites in the | 16495, 16542, 16565, 33, 61, 78, 80, 91, 96, |
| D-loop region | 105, 120, 162, 170 and 186. |

TABLE 2
List of CpG sites from 3284 and 3657 in the ND1 gene.

| | |
|---|---|
| CpG sites in the | 3351, 3375, 3379, 3406, 3420, 3435, 3453, |
| ND1 gene | 3459, 3495, 3525, 3530, 3549 and 3642. |

TABLE 3
List of CHG sites from 16491 and 202 in the D-loop region.

| | |
|---|---|
| CHG sites in the | 16494, 16501, 16514, 6, 64, 98, 104, 122, 128, |
| D-loop region | 141 and 182. |

TABLE 4
List of CHG sites from 3284 and 3657 in the ND1 gene.

| CHG sites in the | 3374, 3455, 3494, 3524, 3529, 3589, 3641 and |
|---|---|
| ND1 gene | 3657. |

TABLE 5
List of CHH sites from 16491 and 202 in the D-loop region.

| | |
|---|---|
| CHH sites in the | 16498, 16507, 16508, 16511, 16520, 16527, |
| D-loop region | 16528, 16536, 16537, 16538, 16540, 16546, |
| | 16547, 16548, 16549, 16560, 16563, 4, 11, 15, |
| | 17, 18, 19, 26, 27, 29, 31, 39, 41, 43, 44, 48, 76, |
| | 86, 110, 112, 113, 114, 132, 140, 144, 145, 147, |
| | 150, 151, 164, 166, 167, 174, 190, 194 and 198. |

TABLE 6
List of CHH sites from 3284 and 3657 in the ND1 gene.

| | |
|---|---|
| CHH sites in the | 3287, 3292, 3293, 3295, 3298, 3303, 3306, 3310, |
| ND1 gene | 3311, 3312, 3317, 3318, 3321, 3322, 3324, 3325, |
| | 3328, 3330, 3331, 3333, 3340, 3341, 3342, 3346, |
| | 3353, 3359, 3363, 3364, 3370, 3388, 3393, 3400, |
| | 3403, 3408, 3414, 3415, 3416, 3417, 3429, 3430, |
| | 3431, 3432, 3439, 3442, 3445, 3448, 3449, 3450, |
| | 3461, 3462, 3469, 3471, 3474, 3476, 3477, 3484 |
| | 3485, 3486, 3487, 3493, 3497, 3498, 3500, 3503, |
| | 3506, 3507, 3510, 3512, 3513, 3514, 3516, 3519, |
| | 3522, 3527, 3528, 3533, 3534, 3539, 3541, 3543, |
| | 3545, 3546, 3551, 3553, 3556, 3559, 3566, 3567, |
| | 3568, 3569, 3570, 3571, 3573, 3574, 3575, 3576, |
| | 3580, 3581, 3582, 3585, 3586, 3587, 3588, 3594, |
| | 3597, 3598, 3600, 3603, 3604, 3609, 3610, 3612, |
| | 3613, 3622, 3626, 3627, 3629, 3630, 3632, 3636, |
| | 3637, 3648, 3650, 3654 and 3655. |

Another aspect of the invention is directed to a method, particularly a computer-implemented method, for monitoring the progression of DLB in a subject comprising:
(a) determining in a sample from said subject comprising mitochondrial DNA, the methylation pattern as described herein; and
b) comparing the methylation pattern determined in step (a) with said methylation pattern obtained in an earlier stage of the disease. A risk score higher than the previous risk score is indicative of progression of DLB, and thus, a bad prognosis. The risk score can me monitored e.g., once a year.

In some embodiments, increased hypomethylation in at least one site of said CpG sites in the D-loop region, increased hypomethylation in at least one site of said CHG sites in the D-loop region and/or increased hypomethylation in at least one site of said CHH sites in the D-loop region over time is indicative of the progression of DLB.

Alternatively, this aspect can be formulated as a method, particularly a computer-implemented method, for monitoring and treating a subject suffering from DLB comprising:
(a) administering a treatment for DLB to the subject, and
(b) continuing administration of said treatment to the subject if the treatment is being effective, or
(c) discontinuing administration of said treatment to the subject if said treatment is being ineffective, wherein
(i) no increased hypomethylation in at least one site of said CpG sites in the D-loop region, no increased hypomethylation in at least one site of said CHG sites in the D-loop region and/or no increased hypomethylation in at least one site of said CHH sites in the D-loop region with respect to methylation levels prior to administration of said treatment indicates that the treatment is being effective; and
(ii) increased hypomethylation in at least one site of said CpG sites in the D-loop region, increased hypomethylation in at least one site of said CHG sites in the D-loop region and/or increased hypomethylation in at least one site of said CHH sites in the D-loop region with respect to methylation levels prior to administration of said treatment indicates that the treatment is ineffective.

Another aspect of the invention is directed to a method for monitoring the progression of DLB in a subject comprising,
(a) determining in a sample from said subject comprising mitochondrial DNA, the methylation pattern in the D-loop region, wherein the methylation pattern is determined in at least one site selected from the group consisting of:
(i) the CpG sites of the D-loop region shown in Table 1,
(ii) the CHG sites in the D-loop region shown in Table 3, and
(iii) the CHH sites in the D-loop region shown in Table 5;
(b) combining the methylation pattern of one or more sites determined in step (a), with at least one clinical variable of the subject,
wherein said combining is performed using a classification model for determining a score which correlates to the presence of DLB in the subject; and
(c) comparing the score pattern determined in step (b) with a score obtained in an earlier stage of the disease.

In an embodiment, changes in score over time are associated to progression of the disease. In some embodiments, a higher score over time is indicative of progression of the disease. In another embodiment, a lower score or no difference in said score over time is indicative of non-progression of the disease.

Alternatively, this aspect of the invention can be directed to a method for monitoring and treating a subject suffering from DLB comprising:
(a) administering a treatment for DLB to the subject; and
(b) continuing administration of said treatment to the subject if the treatment is being effective, or
(c) discontinuing administration of said treatment to the subject if said treatment is being ineffective, wherein
(i) a lower or equal score indicates that the treatment is effective; and
(ii) a higher score indicates that the treatment is ineffective.

### Subjects

In some embodiments, the subject is an animal, particularly a mammalian. In a particular embodiment, the subject is selected from the group consisting of rat, mouse, cat, dog, chimpanzee, and human. More particularly, the subject is a human.

In some embodiments, the subject is suspected of having dementia. Particularly, the subject is suspected of having DLB. In another embodiment, the subject is at risk of developing or has been diagnosed with dementia. In some embodiments, the subject has at least one symptom of mild dementia. Particularly, the subject has at least one symptom selected from the group consisting of mild memory loss, mild loss of attention capacity, difficulties with reasoning, planning or problem-solving, difficulties in language and declined visual depth perception. In other embodiments, the subject has at least one symptom selected from the group consisting of parkinsonism, visual and/or auditory hallucinations and REM sleep behavior disorder.

In some embodiments, the subject has been diagnosed with a dementia other than DLB. Particularly, the subject has been incorrectly diagnosed with a dementia other than DLB. In some embodiments, the subject is suspected of having an incorrect diagnose. In some embodiments, the subject has a Clinical Dementia Rating (CDR) score of 0.5. Particularly, the subject is diagnosed with Mild Cognitive Impairment. In another embodiment, the subject has a Clinical Dementia Rating Score higher than 0.5. Particularly, the subject has a CDR of 1. In another embodiment, the subject has a CDR higher than 1. Particularly, the subject has a CDR of 2 or 3.

“Clinical Dementia Rating” is a global summary obtained through a semi-structured interview of patients and informants, wherein the subject's cognitive status is rated in six domains of functioning including memory, orientation, judgment and problem solving, community affairs, home and hobbies, and personal care. Each domain is assigned a score between 0 and 3, and the results are computed via an algorithm to obtain a CDR global score. The CDR global score ranges from 0 to 3 and allows for the grouping of subjects according to the severity of their dementia, wherein CDR=0 corresponds to absent cognitive impairment, CDR=0.5 is questionable or very mild dementia, CDR=1 is Mild Dementia, CDR=2 is Moderate Dementia, and CDR=3 is Severe Dementia.

Subjects with a CDR of 0.5 are diagnosed with Mild Cognitive Impairment (MCI), which is an early stage of memory loss or other cognitive ability loss, such as language or visual/spatial perception, in subjects who maintain the ability to independently perform most activities of daily living. Subjects with a CDR of 1 or over are considered to have already progressed to dementia.

Methylation Pattern and their Measurements

### Methylation Patterns

The methylation pattern of the mitochondrial DNA sites described herein can be determined using any method known in the art or future methods developed in the art. For example, mtDNA methylation can be determined by treating samples with bisulfite and sequencing the treated samples.

In some embodiments, determining the methylation pattern comprises determining the methylation pattern in the D-loop region in at least one site selected from the group consisting of:
(i) the CpG sites in the D-loop region shown in Table 1,
(ii) the CHG sites in the D-loop region shown in Table 3,
(iii) the CHH sites in the D-loop region shown in Table 5.

In some embodiments, determining the methylation pattern comprises determining the methylation pattern in the ND1 region in at least one site selected from the group consisting of:
(iv) the CpG sites of the ND1 gene shown in Table 2,
(v) the CHG sites in the ND1 gene shown in Table 4, and
(vi) the CHH sites in the ND1 gene shown in Table 6.

In some embodiments, determining the methylation pattern comprises determining the methylation pattern of at least one of the CpG sites of D-loop region in Table 1. In some embodiments, determining the methylation pattern comprises determining the methylation pattern of at least five CpG sites of D-loop region in Table 1. In some embodiments, determining the methylation pattern comprises determining the methylation pattern of at least ten CpG sites of D-loop region in Table 1. In some embodiments, determining the methylation pattern comprises determining the methylation pattern of all of the CpG sites of D-loop region in Table 1.

In some embodiments, determining the methylation pattern comprises determining the methylation pattern of at least one of the CHG sites of D-loop region in Table 3. In some embodiments, determining the methylation pattern comprises determining the methylation pattern of at least five CHG sites of D-loop region in Table 3. In some embodiments, determining the methylation pattern comprises determining the methylation pattern of at least ten CHG sites of D-loop region in Table 3. In some embodiments, determining the methylation pattern comprises determining the methylation pattern of all of the CHG sites of D-loop region in Table 3.

In some embodiments, determining the methylation pattern comprises determining the methylation pattern of at least one of the CHH sites of D-loop region in Table 5. In some embodiments, determining the methylation pattern comprises determining the methylation pattern of at least five CHH sites of D-loop region in Table 5. In some embodiments, determining the methylation pattern comprises determining the methylation pattern of at least ten CHH sites of D-loop region in Table 5. In some embodiments, determining the methylation pattern comprises determining the methylation pattern of at least twenty-five CHH sites of D-loop region in Table 5. In some embodiments, determining the methylation pattern comprises determining the methylation pattern of at least fifty CHH sites of D-loop region in Table 5. In some embodiments, determining the methylation pattern comprises determining the methylation pattern of all of the CHH sites in the D-loop region shown in Table 5.

In some embodiments, determining the methylation pattern comprises determining the methylation pattern of at least one of the CHG sites of D-loop region in Table 3. Particularly, determining the methylation pattern comprises determining the methylation pattern of all of the CHG sites of D-loop region in Table 3. In a particular embodiment, hypomethylation in at least one site of said CHG sites in the D-loop region is indicative that the subject suffers from DLB. In some embodiments, determining the methylation pattern further comprises determining the methylation pattern of at least one of the CHH sites of D-loop region in Table 5 and/or of at least one of the CpG sites of D-loop region in Table 1. Particularly, determining the methylation pattern further comprises determining the methylation pattern of all of the CHH sites of D-loop region in Table 5 and/or of all CpG sites of D-loop region in Table 1. In a particular embodiment, hypomethylation in at least one site of said CHH sites in the D-loop region and hypomethylation in at least one site of said CpG sites in the D-loop region is indicative that the subject suffers from DLB.

In some embodiments, determining the methylation pattern comprises determining the methylation pattern of at least one of the CpG sites of ND1 gene in Table 2. In some embodiments, determining the methylation pattern comprises determining the methylation pattern of at least five CpG sites of ND1 gene in Table 2. In some embodiments, determining the methylation pattern comprises determining the methylation pattern of at least ten CpG sites of ND1 gene in Table 2. In some embodiments, determining the methylation pattern comprises determining the methylation pattern of all of the CpG sites of ND1 gene in Table 2.

In some embodiments, determining the methylation pattern comprises determining the methylation pattern of at least one of the CHG sites of ND1 gene in Table 4. In some embodiments, determining the methylation pattern comprises determining the methylation pattern of at least five CHG sites of ND1 gene in Table 4. In some embodiments, determining the methylation pattern comprises determining the methylation pattern of all of the CHG sites of ND1 gene in Table 4.

In some embodiments, determining the methylation pattern comprises determining the methylation pattern of at least one of the CHH sites of ND1 gene in Table 6. In some embodiments, determining the methylation pattern comprises determining the methylation pattern of at least five CHH sites of ND1 gene in Table 6. In some embodiments, determining the methylation pattern comprises determining the methylation pattern of at least ten CHH sites of ND1 gene in Table 6. In some embodiments, determining the methylation pattern comprises determining the methylation pattern of at least twenty-five CHH sites of ND1 gene in Table 6. In some embodiments, determining the methylation pattern comprises determining the methylation pattern of at least fifty CHH sites of ND1 gene in Table 6. In some embodiments, determining the methylation pattern comprises determining the methylation pattern of all of the CHH sites in the ND1 gene shown in Table 6.

In some embodiments, determining the methylation pattern comprises determining the methylation pattern of at least one of the CHH sites of ND1 gene in Table 6. Particularly, determining the methylation pattern comprises determining the methylation pattern of all of the CHH sites of ND1 gene in Table 6. In a particular embodiment, no significant differences on methylation levels in at least one site of said CHH sites in the ND1 gene is indicative that the subject suffers from DLB: Alternatively, hypomethylation in at least one site of said CHH sites in the ND1 gene is indicative that the subject suffers from DLB. In some embodiments, determining the methylation pattern further comprises determining the methylation pattern of at least one of the CpG sites of ND1 gene in Table 2 and/or of at least one of the CHG sites of ND1 gene in Table 4. Particularly, determining the methylation pattern further comprises determining the methylation pattern of all CpG sites of ND1 gene in Table 2 and/or of all CHG sites of ND1 gene in Table 4. In a particular embodiment, no significant differences on methylation levels in at least one site of said CpG sites in the ND1 gene and no significant differences on methylation levels in at least one site of said CHG sites in the ND1 gene is indicative that the subject suffers from DLB.

In some embodiments, determining the methylation pattern comprises determining methylation in all CpG, CHG and CHH sites of the D-loop region shown in Tables 1, 3 and 5.

In some embodiments, determining the methylation pattern comprises determining methylation in all CpG, CHG and CHH sites of the ND1 gene shown in Tables 2, 4 and 6.

In some embodiments, determining the methylation pattern comprises determining the methylation pattern of all CpG, CHG and CHH sites of the D-loop region and ND1 gene.

The methylation pattern can be determined by any method known in the art. In some embodiments, the methylation pattern is determined by a technique selected from the group consisting of techniques based on bisulfite treatment, techniques based on biological identification, and bisulfite-free and enzyme-free techniques.

In some embodiments, techniques based on bisulfite treatment include but are not limited to sequence-based analysis, analysis based on melting temperature and interaction-based analysis. In some embodiments, sequence-based analysis include but are not limited to bisulfite sequencing, methylation specific PCR (MS-PCR), methylation-sensitive single-nucleotide primer extension (Ms-SnuPE) and reduced representation bisulfite sequencing (RRBS). In some embodiments, analysis based on melting temperature include but are not limited to methylation-specific denaturing gradient gel electrophoresis (MS-DGGE), methylation-specific melting curve analysis (MS-MCA), methylation-specific high-resolution melting (MS-HRM). In some embodiments, interaction-based analysis includes but are not limited to combined bisulfite-restriction analysis (COBRA) and Methylight assay.

In some embodiments, techniques based on biological identification include but are not limited to methods based on enzymatic digestion and bio-dependence reactions. In some embodiments, methods based on enzymatic digestion include but are not limited to Restriction-landmark genomic scanning (RLGS), online monitoring and Methylation sensitive restriction enzyme-PCR (MS-RE-PCR/Southern). In a particular embodiment, the bio-dependence reaction is Methyl capture using methyl-CpG binding domain (MBD) proteins.

In some embodiments, bisulfite-free and enzyme-free techniques include but are not limited to analysis based on direct oxidation and analysis based on the chemical decomposition of oxidation. In a particular embodiment, the analysis based on direct oxidation is choline chloride monolayer supported multiwalled carbon nanotubes (MWCNTs/Ch/GCE). In a particular embodiment, the analysis based on the chemical decomposition of oxidation is Na1O4/LiBr.

In a particular embodiment, the methylation pattern is determined by a technique based on bisulfite treatment. Particularly, the methylation pattern is determined by a sequence-based analysis. More particularly, the methylation pattern is determined by bisulfite sequencing.

In some embodiments, the methylation pattern is determined by a sequencing technique selected from the group consisting of methylation specific PCR (MS-PCR), quantitative methylation specific polymerase chain reaction (qMSP), bisulfite sequencing, pyrosequencing, nanopore sequencing, MassArray, methylation-sensitive single-nucleotide primer extension (Ms-SnuPE), reduced representation bisulfite sequencing (RRBS), methylation-specific denaturing gradient gel electrophoresis (MS-DGGE), methylation-specific melting curve analysis (MS-MCA), methylation-specific high resolution melting (MS-HRM), combined bisulfite-restriction analysis (COBRA) and Methylight assay, methylation-specific restriction endonucleases analysis (MSRE), methylation-sensitive restriction enzyme sequencing (MRE-seq), Restriction-landmark genomic scanning (RLGS), methylated DNA Immunoprecipitation MeDIP or MeDIP-seq, methyl capture using methyl-CpG binding domain (MBD) proteins, ChIP assays, methylation arrays, choline chloride monolayer supported multiwalled carbon nanotubes (MWCNTs/ch/CGE) and analysis based on the chemical decomposition of oxidation (NalO4/LiBr).

In a particular embodiment, the methylation pattern is determined by bisulfite sequencing. In some embodiments, bisulfite sequencing comprises a step of treating the sample with bisulfite and another step of sequencing the bisulfite-treated sample by PCR. In a particular embodiment, the bisulfite-treated sample is sequenced using kits which can be but are not limited to kits produced by Illumina. More particularly, the bisulfite-treated sample is sequenced using a kit selected from the group consisting of MiSeq reagent Kit v3-600 cycles (#MS-102-3003, Illumina), MiSeq reagent Kit v2-500-cycles (#MS-102-2003, Illumina) and MiSeq reagent Nano Kit v2-500 cycles (#MS-103-1003, Illumina).

Sequencing of samples can be performed using any known method in the art. Platforms of sequencing include but are not limited to Roche, Illumina, Life Technologies, Polonator, Helicos Bioscience, Pacific Biosciences, HTG Molecular Diagnostic, Singular Genomics, Element Biosciences, Oxford Nanopore and Nanostring Technology.

In some embodiments, determining the methylation pattern comprises a step of library quantification. In some embodiments, library quantification is performed using a fluorometric quantification method. Alternatively, quantification of the methylation pattern is determined using fluorescence. In a particular embodiment, the fluorometric quantification method is characterized in using kits comprising dsDNA binding dyes. In a particular embodiment, library quantification is performed using the Qubit® 3.0 Fluorometer produced by Thermofisher Scientific or other kits produced by Thermofisher Scientific. More particularly, library quantification is performed using a Qubit™ dsDNA Assay Kit. Particularly, the Qubit™ dsDNA Assay Kit is selected from the group consisting of Qubit™ dsDNA HS Assay Kit #Q32854 and Qubit™ dsDNA BR Assay Kit #Q32850.

It is understood that library quantification using a fluorometric quantification method in the present invention can be performed using fluorometers and kits comprising dsDNA binding dyes from other brands, beyond Thermofisher Scientific: An example of another suitable fluorometer would be but is not limited to QFX Fluorometer produced by DeNovix and Quantus™ Fluorometer produced by Promega. An example of other suitable dsDNA Fluorescence kits are but are not limited to QuantiFluor® Dye Systems and QuantiFluor® dsDNA produced by Promega. Further, QFX Fluorometer from DeNovix works with any of its own DeNovix dsDNA Fluorescence Quantification Kits and other common commercially available assays.

In the examples of the present invention, the analysis to compare methylation levels in each methylation site is conducted using DSS (Dispersion Shrinkage for Sequencing data) Bioconductor package. In other embodiments, any other adequate methodology known in the art can be used. In some embodiments, the analysis is performed using beta-binominal based models. In a particular embodiment, the model is a beta-binominal generalized linear model with a logit link function. In another particular embodiment, the model conducts a local smoothing within each sample, and then applies a beta regression with a probit link function. In some embodiments, the analysis is performed using alternative approaches applying empirical Bayes methods, standard maximum likelihood or other methods known in the art. In some embodiments, the analysis comprises conducting inference using standard techniques known in the art or other suitable techniques developed in the future. In a particular embodiment, inference is conducted using a standard technique selected from the group consisting of Wald tests, likelihood ratio tests, permutation tests and ANOVA.

In the examples of the present invention, the threshold adjusted p-value to establish differential methylation is 0.05. In other embodiments, the threshold adjusted p-value is stablished at 0.25. In other embodiments, the threshold adjusted p-value is stablished at 0.2, In other embodiments, the threshold adjusted p-value is stablished at 0.15. In other embodiments, the threshold adjusted p-value is stablished at 0.1. In other embodiments, the threshold adjusted p-value is stablished at 0.09. In other embodiments, the threshold adjusted p-value is stablished at 0.08. In other embodiments, the threshold adjusted p-value is stablished at 0.07. In other embodiments, the threshold adjusted p-value is stablished at 0.06. In other embodiments, the threshold adjusted p-value is stablished at 0.05. In other embodiments, the threshold adjusted p-value is stablished at 0.04. In other embodiments, the threshold adjusted p-value is stablished at 0.03. In other embodiments, the threshold adjusted p-value is stablished at 0.02. In other embodiments, the threshold adjusted p-value is stablished at 0.01.

### Samples and Sample Processing

The terms “biological sample” or “sample” as used herein refer to biological material isolated from a subject. The biological sample can contain any biological material suitable for determining methylation patterns, e.g., by treating and sequencing nucleic acids.

In some embodiments, the sample is selected from a biofluid or biopsy of a solid tissue. Particularly, the sample is selected from the group consisting of blood, plasma, saliva, cerebrospinal fluid, brain sample, skin sample and urine. Particularly, the sample is blood, particularly peripheral blood.

The source of the sample can be a solid tissue, e.g., from a fresh, frozen and/or preserved organ, tissue sample, biopsy, or aspirate. In some embodiments, the sample is a cell-free sample, e.g., comprising cell-free nucleic acids (e.g., DNA or RNA). A sample can, in some embodiments, comprise compounds that are not naturally intermixed with the tissue in nature such as preservatives, anticoagulants, buffers, fixatives, nutrients, antibiotics or the like.

In some embodiments, the method comprises obtaining the sample. In a particular embodiment, the sample is blood or plasma, and the sample is extracted by using a needle. In another embodiment, the sample is saliva, and the sample is obtained using a method selected from the group consisting of draining method, spitting method, suction method and swab method. In some embodiments, the sample can be obtained, e.g., from surgical material or from biopsy. In some embodiments, the biopsy can be archival tissue from a previous line of therapy. In some embodiments, the biopsy can be from tissue that is therapy naive.

In some embodiments, the sample is frozen or preserved. In some embodiments, the sample is preserved as a frozen sample or as formalin-, formaldehyde-, or paraformaldehyde-fixed paraffin-embedded (FFPE) tissue preparation. For example, the sample can be embedded in a matrix, e.g., an FFPE block or a frozen sample. In some embodiments, a sample can comprise bone marrow; aspirates; scrapings; bone marrow specimens; tissue biopsy specimens; surgical specimens; etc. In some embodiments, a sample is or comprises cells obtained from an individual, e.g., from an individual from whom the sample is obtained.

In some embodiments, samples are fresh samples (or non-archival samples) or archival samples. As used herein, the terms “fresh sample,” “non-archival sample,” and grammatical variants thereof refer to a sample which has been processed before a predetermined period of time, e.g., one week, after extraction from a subject. In some embodiments, a fresh sample has not been frozen. In some embodiments, a fresh sample has not been fixed. In some embodiments, a fresh sample has been stored for less than about two weeks, less than about one week, or less than six, five, four, three, or two days before processing. As used herein, the term “archival sample” and grammatical variants thereof refers to a sample which has been processed after a predetermined period of time, e.g., a week, after extraction from a subject. In some embodiments, an archival sample has been frozen. In some embodiments, an archival sample has been fixed. In some embodiments, an archival sample has a known diagnostic and/or a treatment history. In some embodiments, an archival sample has been stored for at least one week, at least one month, at least six months, or at least one year, before processing.

### Oligonucleotides and Kits

In some embodiments, in any method described herein, the methylation pattern is determined using at least one oligonucleotide capable of specifically hybridizing with a mitochondrial DNA comprising the D-loop region and/or the ND1 gene. Particularly, oligonucleotides are capable of specifically hybridizing under high stringency conditions.

The sequence of interest can refer to the reference sequence or the sequence resulting from certain modification treatment, e.g., bisulfite treatment wherein unmethylated cytosines are modified to uracil. In some embodiments, the oligonucleotide hybridizes the reference mitochondrial DNA sequence comprising the D-loop region or the ND1 gene. In a particular embodiment, the oligonucleotide hybridizes a modified mitochondrial DNA sequence comprising the D-loop region. Alternatively, the oligonucleotide hybridizes a modified mitochondrial DNA sequence comprising the ND1 gene. In a particular embodiment, the modified mitochondrial DNA sequence has been modified by bisulfite treatment. In a more particular embodiment, the modified mitochondrial DNA sequence has been modified by bisulfite treatment, wherein non-methylated cytosines have been modified to uracil.

In some embodiments, the methylation pattern is determined using at least one oligonucleotide capable of specifically hybridizing with a mitochondrial DNA sequence comprising at least one methylation site selected from the group consisting of:
(i) the CpG sites in the D-loop region shown in Table 1,
(ii) the CHG sites in the D-loop region shown in Table 3,
(iii) the CHH sites in the D-loop region shown in Table 5, (iv) the CpG sites of the ND1 gene shown in Table 2,
(iv) the CHG sites in the ND1 gene shown in Table 4, and
(v) the CHH sites in the ND1 gene shown in Table 6.

Alternatively, the oligonucleotides of the invention are referred to as nucleic acid sequences. In some embodiments, the oligonucleotides are DNA sequences. In some embodiments, the oligonucleotides have a length between 15 and 100 nucleotides.

In an embodiment, the methylation pattern is determined using oligonucleotides capable of specifically hybridizing and amplifying a mitochondrial DNA sequence comprising nucleotides from 16,465 to 230 of NCBI Reference Sequence: NC_012920.1 (corresponding to D-loop region) and/or with a mitochondrial DNA sequence comprising nucleotides from 3,257 to 3,682 of NCBI Reference Sequence: NC_012920.1 (corresponding to ND1 gene).

Inventors herein designed primers (also herein referred to as oligonucleotides or nucleic acid sequences) which included the least number of cytosines. In some embodiments, the primers are degenerated to cover all possible methylated and non-methylated scenarios due to the uncertain C/U conversion of the few cytosines residues included in the sequences. These primers are a mixture of oligonucleotide sequences which contain several possible nucleotide bases at certain position. Consequently, the probability to detect mitochondrial methylation is higher. The forward degenerated primers include Y, which refers to either C or T (Y=C/T), in any position wherein the reference sequence is a C. Further, as known in the art, reverse primers do not correspond to the reference sequence, but to the reversed complementary of the reference sequence. Therefore, the reverse primers do not include C sites of the reference sequence, but their complementary G sites. Thus, reverse degenerated primers include R, which refers to either G or A (R=A/G), in any position wherein the reference sequence is a C, or wherein the complementary sequence includes a G.

Thus, in some embodiments, the oligonucleotides are degenerated oligonucleotides. Degenerated oligonucleotides/primers is a mix of oligonucleotide sequences in which some positions contain a number of possible bases, giving a population of oligonucleotides with similar sequences that cover all possible nucleotide combinations.

In a particular embodiment, the methylation pattern is determined using at least one oligonucleotide with a length between 15 and 100 nucleotides, comprising a nucleic acid sequence selected from the group consisting of SEQ ID NO: 1, SEQ ID NO: 2, SEQ ID NO: 3 and SEQ ID NO: 4. Oligonucleotides can comprise additional nucleotides in their ends to be adequate for using in e.g., sequencing (i.e. sequencing adapters).

In a particular embodiment, the methylation pattern is determined using at least one oligonucleotide selected from the group consisting of SEQ ID NO: 1, SEQ ID NO: 2, SEQ ID NO: 3 and SEQ ID NO: 4. In a more particular embodiment, the methylation pattern is determined using the oligonucleotides SEQ ID NO: 1, SEQ ID NO: 2, SEQ ID NO: 3 and SEQ ID NO: 4. In another embodiment, the methylation pattern is determined using at least one oligonucleotide selected from SEQ ID NO: 1 and SEQ ID NO: 2. In some embodiments, the methylation pattern is determined using oligonucleotides SEQ ID NO: 1 and SEQ ID NO: 2.

Another aspect of the invention relates to an oligonucleotide with a length between 15 and 100 nucleotides, comprising a nucleic acid sequence selected from the group consisting of SEQ ID NO: 1, SEQ ID NO: 2, SEQ ID NO: 3 and SEQ ID NO: 4. As said, in an embodiment, the oligonucleotide comprises sequencing adapters at the ends of SEQ ID NO: 1, 2, 3 and/or 4. Particularly, the oligonucleotide is selected from the group consisting of SEQ ID NO: 1, SEQ ID NO: 2, SEQ ID NO: 3, and SEQ ID NO: 4. In a particular embodiment, the nucleic acid sequence is SEQ ID NO: 1. In another particular embodiment, the oligonucleotide is SEQ ID NO: 2. In another particular embodiment, the oligonucleotide is SEQ ID NO: 3. In another particular embodiment, the oligonucleotide is SEQ ID NO: 4.

Another aspect of the invention relates to the use of an oligonucleotide with a length between 15 and 100 nucleotides, comprising a nucleic acid sequence selected from the group consisting of SEQ ID NO: 1, SEQ ID NO: 2, SEQ ID NO: 3 and SEQ ID. NO: 4, for the determination of a methylation pattern of mitochondrial DNA. Particularly, the oligonucleotide is selected from the group consisting of SEQ ID NO: 1, SEQ ID NO: 2, SEQ ID NO: 3, and SEQ ID NO: 4. In a particular embodiment, the invention relates to the use of an oligonucleotide with SEQ ID NO: 1 for the determination of a methylation pattern of a mitochondrial DNA sequence comprising the D-loop region. In another embodiment, the invention relates to the use of an oligonucleotide with SEQ ID NO: 2 for the determination of a methylation pattern of a mitochondrial DNA sequence comprising the D-loop region. In another embodiment, the invention relates to the use of an oligonucleotide with SEQ ID NO: 3 for the determination of a methylation pattern of a mitochondrial DNA sequence comprising the ND1 gene. In another embodiment, the invention relates to the use of an oligonucleotide SEQ ID NO: 4 for the determination of a methylation pattern of a mitochondrial DNA sequence comprising the ND1 gene.

In some embodiments, the invention relates to the use of an oligonucleotide selected from SEQ ID NO: 1 and SEQ ID NO: 2 for the determination of a methylation pattern of a mitochondrial DNA sequence comprising the D-loop region. In another embodiment, the invention relates to the use of a oligonucleotide selected from SEQ ID NO: 1 and SEQ ID NO: 2 for the determination of a methylation pattern to diagnose or identify DLB in a subject.

In other embodiments, the invention relates to the use of an oligonucleotide with a length between 15 and 100 nucleotides, comprising a nucleic acid sequence selected from the group consisting of SEQ ID NO: 1, SEQ ID NO; 2, SEQ ID NO: 3 and SEQ ID NO: 4 (and particularly with SEQ ID NO: 1, SEQ ID NO: 2, SEQ ID NO: 3, and SEQ ID NO: 4), for the determination of a methylation pattern of a mitochondrial DNA sequence comprising the D-loop region and/or the ND1 gene. In another embodiment, the invention relates to the use of said oligonucleotides for the determination of a methylation pattern to diagnose or identify DLB in a subject.

In some embodiments, the oligonucleotides defined herein are included in a kit. In those lines, another aspect of the invention relates to the use of the kit for the determination of a methylation pattern of mitochondrial DNA. In another aspect, the invention relates to the use of the kits as defined above for the determination of a methylation pattern of mitochondrial DNA to diagnose or identify DLB in a subject. In another aspect, the invention relates to the use of the kits as defined above, following the methods as described herein.

Such kits can comprise containers, each with one or more of the various reagents (e.g., in concentrated form) utilized in the method, including, e.g., one or more oligonucleotides (e.g., oligonucleotides with SEQ ID NO: 1-4 provided herein, particularly oligonucleotides with SEQ ID NO: 1-2 provided herein). The kit can also provide reagents, buffers, and/or instrumentation to support the practice of the methods provided herein.

A kit provided according to the invention can also comprise brochures or instructions describing the methods disclosed herein or their practical application to diagnose or identify DLB in a subject. Instructions included in the kits can be affixed to packaging material or can be included as a package insert. While the instructions are typically written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions.

In some embodiments, the kit is an Illumina sequencing kit. More particularly, the kit is selected from the group consisting of MiSeq reagent Kit v3-600-cycles (#MS-102-3003, Illumina), MiSeq reagent Kit v2-500-cycles (#MS-102-2003, Illumina) and MiSeq reagent Nano Kit v2-500 cycles (#MS-103-1003, Illumina).

### Clinical Variables

The methods described herein are capable of diagnosing or identifying DLB in a subject. Information resulting from clinical variables of the subject can be used in the methods described herein to improve or complement the diagnosis. Further, clinical variables can be used as input data in the classification model described herein. Moreover, the classification model can be trained with a training dataset which comprises clinical variables associated with a plurality of subjects.

Thus, in some embodiments, any method described herein, further comprises considering at least one clinical variable of the subject. In other embodiments, any method described herein, further comprises combining with the methylation patterns described herein at least one clinical variable of the subject. Particularly, any method of the invention further comprises considering at least one clinical variable of the subject to identify or diagnose DLB in a subject, or to identify a subject with DLB.

Clinical variables can include any clinical variable known in the art. In some embodiments, the method comprises considering at least one clinical variable of the subject or combining the methylation patterns described herein with at least one clinical variable of the subject. In some embodiments, the clinical variables are selected from the group consisting of demographic variables, neuropsychological variables, clinical observations variables and clinical tests variables.

### Demographic Variables

“Sex” is a categorical variable comprising two possible categories: Female or Male.

“Age” is a numerical variable which corresponds to the years of the subject.

“Race” is a categorical variable comprising two or more possible categories: white or Caucasian, black, American Indian, Latino or Hispanic, Asian, etc. “Race” is a category of humankind that share certain distinctive physical traits.

“Scholarship” is a numerical variable which corresponds to the years of education from the age of 6.

“Family history” is a categorical variable comprising two possible categories: familiar antecedents of dementia or no familiar antecedents of dementia.

In an embodiment, the demographic variables are selected from sex, age, race, scholarship and family history.

### Neuropsychological Variables

Neuropsychological testing may be useful to stratify the level of cognitive impairment and when the clinician suspects a diagnosis of DLB or is trying to differentiate DLB from other types of dementia. Considering the cognitive profile of DLB, neuropsychological assessment should focus on attention, executive functions, visuoperceptual abilities, language functions, and memory (immediate and delayed recall). The neuropsychological tests can vary at each center/country but they evaluate the same cognitive functions.

Some of the neuropsychological tests are Clinical Dementia Rating (CDR), Clinical Dementia Rating Scale Sum of Boxes (CDR-SOB), Mini-Mental State Exam (MMSE), Montreal Cognitive Assessment (MoCA), Global Deterioration Scale (GDS), Functional Activities Questionnaire (FAQ), Geriatric Depression Scale Yesavage (GDS-Yesavage), Neuropsychiatric Inventory score (NPI), Delayed Verbal Memory score, Verbal Learning Curve score, Verbal Recognition score, Semantic Verbal Fluency score, Constructional Praxis score, Clock drawing test-Luria.

“MMSE” (Mini-Mental State Exam) is a numerical variable, which refers to an evaluation of five main items: orientation, fixation, concentration and calculation, memory and language, and construction with an output score between 1 and 30.

The following neuropsychological variables can be considered in cases where the subject has been diagnosed with MCI and the diagnosis is unclear: CDR is a numerical variable which refers to “Clinical Dementia Rating”, which is a global summary obtained through a semi-structured interview of patients and informants, and the subject's cognitive status is rated in six domains of functioning including memory, orientation, judgment, and problem solving, community affairs, home and hobbies, and personal care. Each domain is assigned a score between 0 and 3, and the results are computed via an algorithm to obtain a CDR global score. The CDR global score ranges from 0 to 3 and allows for the grouping of subjects according to the severity of their dementia, wherein CDR=0 corresponds to absent cognitive impairment, CDR=0.5 is questionable or very mild dementia, CDR=1 is Mild Dementia, CDR=2 is Moderate Dementia, and CDR=3 is Severe Dementia.

Subjects with a CDR of 0.5 are diagnosed with MCI, which is an early stage of memory loss or other cognitive ability loss, such as language or visual/spatial perception, in subjects who maintain the ability to independently perform most activities of daily living.

“CDR-SOB” (Clinical Dementia Rating Scale Sum of Boxes) is a numerical variable, which refers to “Sum of Boxes Score”, a score ranging from 0 to 18 obtained by summing each of the domain box scores described above for the calculation of CDR global score.

In an embodiment, the neuropsychological variables are selected from the group consisting of MMSE, CDR and CDR-SOB. More particularly, the neuropsychological variable is MMSE.

In one embodiment, the neuropsychological variables are praxis tests, Luria's tests, CDR, GDS, MMSE or CDR-SOB.

### Clinical Observations Variables

“Neuroleptic intolerance” is a categorical variable comprising two possible categories: neuroleptic intolerance or non-neuroleptic intolerance. This variable corresponds to the particular sensitiveness of patients with DLB to neuroleptic drugs with respect to developing extrapyramidal symptoms.

“REM sleep behavior disorder” is a categorical variable comprising two possible categories: presence REM sleep behavior disorder or absence REM sleep behavior disorder. REM sleep behavior is a parasomnia with abnormal dream-enacting behavior during the rapid eye movement sleep.

“Dysautonomia” is a categorical variable comprising two possible categories: presence of dysautonomia or absence of dysautonomia. Dysautonomia or autonomic dysfunction is a condition in which the functioning of the autonomic nervous system (ANS) is impaired.

“Parkinsonism” is a categorical variable comprising two possible categories: presence of parkinsonism or absence of parkinsonism. Parkinsonism is a clinical syndrome characterized by tremor, bradykinesia (slowed movements), rigidity, and postural instability.

“Visual hallucinations” is a categorical variable comprising two possible categories: presence of visual hallucinations or absence of visual hallucinations. Visual hallucinations are visual perceptions in the absence of an external visual stimulus that has the qualities of a real visual perception.

“Cognitive fluctuations” is a categorical variable comprising two possible categories: presence of cognitive fluctuations or absence of cognitive fluctuations. Cognitive fluctuations are spontaneous alterations in concentration, attention, alertness, and wakefulness which can vary greatly from day to day, or even throughout the same day.

#####Clinical variables which have been defined herein as categorical variables with only two possible categories can be regarded as categorical variables with more than two options in the case that degrees, grades or ranks of such variable are determined, rather than only the presence or absence of such variable. Similarly, clinical variables which have been defined herein as categorical variables with two or more possible categories can also be regarded as numerical variables.

In an embodiment, the clinical observations variables are neuroleptic intolerance, REM sleep behavior disorder, dysautonomia; parkinsonism, visual hallucinations and cognitive fluctuations.

### Clinical Test Variables

In some embodiments, clinical variables can include clinical test variables, such as tests obtained through neuroimaging techniques or medical imaging techniques. In some embodiments, clinical variables are selected from the group consisting of MRI, DaTSCAN and 18F-FDG-PET.

“MRI” (Magnetic resonance imaging) is a categorical variable comprising two possible categories: positive MRI and negative MRI. MRI is a type of scan that uses strong magnetic fields and radio waves to produce detailed images of the inside of the body.

“DaTSCAN” (Dopamine Transporter Scan) is a categorical variable comprising two possible categories: positive DaTSCAN and negative DaTSCAN is diagnostic method used to determine a loss of dopaminergic neurons in striatum.

Positron emission tomography (PET) is a type of nuclear medicine procedure that measures metabolic activity of the cells of body tissues. PET can be performed with different types of tracers, each of them used for a specific purpose, study or detection. For example, PET can measure glucose levels, beta-amyloid plaques or tau protein. FDG-PET refers to a PET designed to detect de glucose and consequently analyze metabolic activity of tissues or body sites. In another example, PET is used to determine the presence of beta-amyloid plaques in the brain. PET can also be used to measure glucose, beta-amyloid and/or tau.

“18F-FDG-PET” (positron emission tomography with 18F-fluorodeoxyglucose) is a categorical variable comprising two possible categories: positive 18F-FDG-PET and negative 18F-FDG-PET. “18F-FDG-PET” is an imagining metabolic technique which uses a radioactive tracer which refers to a PET designed to detect glucose and consequently analyze metabolic activity of tissues or body sites.

Clinical variables which have been defined herein as categorical variables with only two possible categories can be regarded as categorical variables with more than two options in the case that degrees, grades of ranks of such variable are determined, rather than only the presence or absence of such variable. Similarly, clinical variables which have been defined herein as categorical variables with two or more possible categories can also be regarded as numerical variables.

The following clinical test variables can be considered in cases where the subject has been diagnosed with MCI, however the diagnosis is yet unclear:

“Amyloid PET” is a categorical variable comprising two possible categories: positive amyloid PET and negative amyloid PET, which refer to the presence or absence of beta-amyloid, respectively. Amyloid PET is used to determine the presence of beta-amyloid plaques in the brain.

Clinical variables can include biomarkers known in the art, as well as other neuropsychological tests and other radio imaging techniques known in the art. In some embodiments, clinical variables include the presence, absence or the levels of biomarkers known in the art (e.g., APOE). Particularly, clinical variables include the presence or absence of said biomarkers. In some embodiments, biomarkers can be measured in any sample or fluid from the subject and particularly, in cerebrospinal fluid samples. In other embodiments, biomarkers are measured in blood samples. In other embodiments, biomarkers can be detected using other radio imaging techniques, e.g., positron emission tomography (PET). In some embodiments, clinical variables include APOE, aIE4, β-42, tau-T and/or tau-P.

“APOE” is a categorical variable comprising categories which correspond to the different genotype of Apolipoprotein E: E2.E2, E2.E3, E2.E4, E3.E3, E3.E4 and E4.E4.

“aIE4” is a categorical variable corresponding to the recategorization of APOE genotype into the following categories: 0 (comprising E2.E2, E2.E3 and E3.E3), 1 (comprising E2.E4 and E3.E4) and 2 (comprising E4.E4).

“β-42” is a categorical variable which refers to the presence or absence of β-42 peptide in a sample of cerebrospinal fluid.

“tau-T” is a categorical variable which refers to the presence of absence of protein Tau in a sample of cerebrospinal fluid.

“tau-P” is a categorical variable which refers to the presence of absence of protein Tau phosphorylated in a sample of cerebrospinal fluid.

In some embodiments, the clinical test variables are magnetic resonance imaging (MRI), Dopamine Transporter Scan (DaTSCAN), positron emission tomography with 18F-fluorodeoxyglucose (18F-FDG-PET), amyloid PET, apolipoprotein E (APOE) genotype, aIE4, β-42, tau-T and tau-P.

In some embodiments, clinical variables can include a variable relating to a current treatment or medication of the subject, particularly for dementia or dementia-related symptoms (i.e., concomitant medication).

In some embodiments, the clinical variable is selected from the group consisting of sex, age, race, scholarship, family history, MMSE, neuroleptic intolerance, REM sleep behavior disorder, dysautonomia, parkinsonism, visual hallucinations, cognitive fluctuations, MRI, DaTSCAN and 18F-FDG-PET.

In another embodiment, the clinical variable is selected from the group consisting of CDR, SOB, amyloid PET, APOE, aIE4, β-42, tau-T and tau-P.

In some embodiments, the clinical variable is selected from the group consisting of: sex, age, race, scholarship, family history, praxis tests, Luria's tests, CDR, GDS, MMSE, CDR-SOB, neuroleptic intolerance, REM sleep behavior disorder, dysautonomia, parkinsonism, visual hallucinations, cognitive fluctuations, magnetic resonance imaging (MRI), Dopamine Transporter Scan (DaTSCAN), positron emission tomography with 18F-fluorodeoxyglucose (18F-FDG-PET), amyloid PET, apolipoprotein E (APOE) genotype, aIE4, β-42, tau-T and tau-P.

In a particular embodiment, the clinical variable is selected from the group consisting of age, CDR, GDS, REM sleep behavior disorder, dysautonomia, parkinsonism, visual hallucinations, cognitive fluctuations, PET-FDG and DatTSCAN.

### Classification Model

In another aspect, the present invention provides a classification model that is capable of classifying subjects in two categories: subjects with DLB and subjects without DLB. These classes are associated to a subject suffering from DLB and subjects who do not duffer from DLB. Subjects that are not classified as having DLB can be healthy subjects, subjects at earlier stages of dementia, or subjects suffering from other types of dementia or other diseases.

Thus, the present invention provides a classification model for identifying DLB in a subject, wherein the classification model identifies a subject as pertaining to a class from the group consisting of subjects with DLB and subjects without DLB, using as data the methylation patterns obtained from a sample from the subject and/or data on clinical variables of the subject, and wherein being identified as subject with DLB indicates that the subject suffers from DLB. In some embodiments, the classification model identifies a subject as pertaining to a class from the group consisting of subjects with DLB and subjects without DLB, using as data the methylation patterns obtained from a sample from the subject, and wherein being identified as subject with DLB indicates that the subject suffers from DLB. In other embodiments, the classification model identifies a subject as pertaining to a class from the group consisting of subjects with DLB and subjects without DLB, using as data the clinical variables of the subject, and wherein being identified as subject with DLB indicates that the subject suffers from DLB.

Thus, the invention provides a method for identifying dementia with Lewy bodies in a subject, comprising:
(a) determining in a sample of the subject comprising mitochondrial DNA, the methylation pattern in the D-loop region and/or the ND1 gene of the mitochondrial DNA, wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6; and
(b) combining the methylation pattern of one or more sites determined in step (a), optionally with at least one clinical variable of the subject, wherein said combining is performed using a classification model for determining a score which correlates to the identification of dementia with Lewy Body in the subject:

In some embodiments, the classification model is obtained by methods of artificial intelligence. In a particular embodiment, the classification model is obtained by machine learning methods. In a more particular embodiment, the classification model is obtained by a supervised machine learning method or related software. In a more particular embodiment, the supervised machine learning method of related software is selected from the group consisting of Linear Discriminant Analysis (LDA), Classification and Regression Trees (CART), k-Nearest Neighbors (kNN), Naive Bayes (NB), Support Vector Machines (SVM) with a linear kernel, Random Forest (RF), Neural Network (NNET), Generalized Boosted Regression Model (GBM), Binominal Logistic Regression (GLM), Artificial Neural Network (ANN), GBoost (XGB; an implementation of gradient boosted decision trees designed for speed and performance), GLMNET (Generalized Linear Model via Penalized Maximum Likelihood, a package that fits a generalized linear model via penalized maximum likelihood e.g., the implementation of a Logistic Regression)), cforest (implementation of the random forest and bagging ensemble algorithms utilizing conditional inference trees as base learner), Treebag (bagging, i.e., bootstrap aggregating, algorithm to improve model accuracy in regression and classification problems which building multiple models from separated subsets of train data, and constructs a final aggregated model), AdaBoost (AdaBoost Classification Trees), PMR (Penalized Multinomial Regression) or a combination thereof. In another embodiment; the supervised machine learning method is any supervised machine learning method known in the art or any other suitable method developed in the future.

In some embodiments, the classification model is obtained by a supervised machine learning method. Particularly, the supervised machine learning method is selected from the group consisting of LDA, CART, KNN, NB, SVM, with a linear kernel, RF, NNET, GBM, GLM, GLMNET, AdaBoost and PMR. In a particular embodiment, the supervised machine learning method is selected from the group consisting of LDA, CART, KNN, NB, SVM, RF, NNET, GLMNET, AdaBoost and PMR. Particularly, the method is RF, GLMNET, or AdaBoost, and more particularly, RF.

In another embodiment, the classification model is obtained by a non-supervised machine learning method. In a particular embodiment, the non-supervised machine learning method is selected from the group consisting of K-means, K-Medoids, Fuzzy C-Means, Agglomerative, Hierarchical Clustering, Gaussian Mixture Model (GMM), Neural Networks, Hidden Markov Model (HMM), Mean-Shift, DBSCAN Clustering, Apriori algorithm, Principle Component Analysis (PCA), Independent Component Analysis (ICA) Linear Discriminant Analysis (LDA), Singular Value Decomposition (SVD), Linear Semantic Analysis (LSA), t-Distributed Stochastic Neighbor Embedding (t-SNE), Nonlinear Multidimensional Scalling, Principal Curves, k-nearest neighbors (kNN), Locally Kinear Embedding and Autoencoders.

Alternatively, the classification model is obtained by using Deep learning algorithms as a set of Machine Learning methods. In a particular embodiment, the deep learning method is selected from the group consisting of Convolutional Neural Networks (CNNs), Long Short-Term Memory Networks (LSTMs), Recurrent Neural Networks (RNNs), Generative Adversarial Networks (GANs), Radial Basis Function Networks (RBFNs), Multilayer Perceptions (MLPs), Self-Organizing Maps (SOMs), Deep Belief Networks (DBNs), Restricted Boltzmann Machines (RBMs) and autoencoders.

In some embodiments, the classification model is trained or has been trained with a training set comprising mitochondria' methylation patterns for the methylation sites as defined herein in a plurality of samples associated with a plurality of subjects. In a particular embodiment, the training set further comprises clinical variables associated with the plurality of subjects. More particularly, each subject of the training set is assigned a Dementia Stage Classification. In a particular embodiment, the Dementia Stage Classification is selected from the group consisting of control and subject with DLB.

In another embodiment, the classification model is trained or has been trained with a training set comprising clinical variables associated with a plurality of subjects. In a particular embodiment, the training set further comprises mitochondrial methylation patterns for each methylation site in a plurality of samples associated with a plurality of subjects. Particularly, each subject of the training set is assigned a Dementia Stage Classification. More particularly, the Dementia Stage Classification is selected from the group consisting of control and subject with DLB.

In some embodiments, subjects classified as controls are characterized by having a CDR score of 0 and a clinical follow-up longer than 10 years (i.e., more than 10 years of clinical follow-up). In some embodiments, subjects classified as subjects with DLB have been clinically diagnosed with DLB.

In some embodiments, the training dataset comprises correct outputs which correspond to the Dementia Stage Class assigned to each subject, wherein the dementia stage classes are controls and subjects with DLB.

In some embodiments, data is pre-processed or has been pre-processed before the classification model is trained. This step is conducted to ensure and enhance the performance of the model training process. The data pre-processing comprises:
(1) creating dummy variables,
(2) removing zero- and near zero-variance variables,
(3) splitting the data into a training and testing data sets,
(4) centering and scaling,
(5) dimensionality reduction (i.e., identifying and Removing Correlated Variables), and
(6) examining and visualizing the training data set.
(1) Creating dummy variables process is conducted in order to handle categorical data. Basically, each categorical variable is transformed to a numerical variable by creating dummy variables using the called “one-hot encoding” approach (i.e., each new variable is coerced to have a value of either 0 or 1, representing the presence or absence of that attribute). The process is performed to ensure that variables are encoded to be consistent. That is, it is coded to guarantee that there are no linear dependencies between the new attributes and thus avoid the dummy variable trap. Additionally, the data is reviewed to ensure that all categorical 1-coded variables do not show any anomalous linear combinations, if so, redundant variables are removed until eliminating the linear combinations.
(2) Removing zero- and near zero-variance variables process is conducted to remove variables showing a single unique value, and variables having a few unique numeric values that are highly unbalanced. Otherwise, these predictors might cause instability issues during the fitting process or the model crashing,
(3) Data splitting process is performed to randomly split into two main subsets: one for performing the model training (80% of the samples) and other for testing the classification model (20% of the samples). The random sampling process is driven within each class to preserve the overall class distribution of the data. A random seed is considered in order to ensure the reproducibility.
(4) Centering and scaling process is applied on the continuous features (variables) of the training data set with a view to estimate the centering and scaling factors that must be applied on both data sets to generate the normalized data sets for performing the training and testing processes of the classification model.
(5) Dimensionality reduction process is performed to reduce the number of variables (i.e., features or attributes) in the data set while retaining as much as relevant information as possible. In other words, the objective is to remove redundant or irrelevant features with view to improve the efficiency and effectiveness of the learning method applied to build the classification model. In this context there are two main approaches: the feature selection and the feature extraction.
(i) Feature selection techniques: The basic idea of these methods is to select a subset of variables based on some criteria, e.g., by identifying and removing correlated variables. This process is conducted with the purpose of reducing highly correlated variables. To perform this step, a correlation matrix is calculated. Usually, the correlation measure applied is the Pearson's Correlation Coefficient. Then, to detect the highly correlated variables, based on the absolute values of pairwise correlations, if two variables have a high correlation, one looks at the mean absolute correlation of each variable and removes the variable with the largest mean absolute correlation. In this context, usually, the pairwise absolute correlation cutoff can be setup by studying, for example, the linear regression between each pair of variables. However, this approach could fail to reveal additional feature correlations. For this reason, on one hand, other monotonic methods can be also considered to identify the correlation between variables. For example, the non parametric methods Spearman's Rank Correlation and Kendall's Tau Correlation measure the association between each pair of variables that are based on the ranks of the observations rather than their actual values. They are usually applied when there are reasonable suspicious of nonlinear relationship between the attributes (variables), skewed or ordinal measures. Other type of method can be the distance correlation (aka dCor). It is performed to measure the dependence between each pair of variables in a way that is sensitive to nonlinear relationships. Most of these methods are thought to study the relationship between continuous and/or ordinal variables but also there are methods to study the strength of the relationship between a dichotomous variable and a continuous variable (e.g., the Point Biserial Correlation) or when both variables are dichotomous (Phi Coefficient). On the other hand, in order to determine an appropriate threshold for detecting the highly correlated variables, it is recommended to use an objective approach such as a cross-validation. That is, to determine the required cutoff, first, one must split the data into training and testing data sets using a random seed to ensure the reproducibility. Second, train the model using the training data set and evaluate its performance using the testing set. Third, vary the threshold for considering the variables as highly correlated and then repeat the second step for each threshold value. Fourth, select the threshold that generates the best performance on the testing set, such as the one with the highest accuracy (or lowest error rate). Fifth, after having select the cutoff, it must be applied to the entire dataset and retrain the model using all the data.

Other feature selection methods that can be applied are the Genetic Algorithms (GA), Li-regularized logistic regression, Lasso regression or hybrid methods.
(ii) Feature extraction techniques: These approaches generate new variables by combining or applying a transformation on the original features into a reduced dimension space. Some examples are the Principal Components (PCA), Multiple Factor Analysis (MFA), t-SNE or alternatively UMAP and/or Multidimensional Scaling (MDS) approaches, Partial Least Square-Discriminant Analysis (PLS-DA) or Autoencoders.
(6) Examining and visualizing the training data set is a process carried out after having performed the previous preprocessing tasks. Itis a second exploration data analysis (EDA) performed on the training data set and guided to review and check no biases from the original data. Classical statistical descriptive methods are applied (that is univariate, bivariate and multivariate descriptive methods).

In an embodiment, the data pre-processing comprises:
(1) data splitting,
(2) centering and scaling,
(3) identifying and Removing Correlated Variables, and
(4) examining and visualizing the training data set.
(1) In data splitting, the original corrected data is randomly split into to main subsets; one for performing the model training (e.g., 80% of the samples) and other for testing the classification model (e.g., 20% of the samples). The random sampling process is driven within each class to preserve the overall class distribution of the data.
(2) In centering and scaling, continuous variables from the training data set are used to estimate the centering and scaling factors that are applied on both data sets to generate the normalized data sets for performing the training and testing processes of the classification model.
(3) Identifying and removing correlated variables is performed to reduce the level of correlation between variables. A pairwise correlation analysis based e.g., on the Spearman's rank correlation coefficient is performed. For those pairs showing high levels of absolute correlation values (e.g., >0.65), the variable with the largest mean absolute correlation is removed from the data set. Furthermore, a Principal Component Analysis (PCA) can be applied to reduce the dimensionality of the features, e.g., to 10 variables.
(4) Examining and visualizing the training data set comprises applying an exploratory data analysis (EDA) on the training data set in order to review and check no biases from the original data.

The classification models disclosed herein can be trained with data corresponding to a set of samples for which methylation data corresponding to a set of methylation sites has been obtained. For example, a training set comprises methylation pattern data from the methylation sites presented in Tables 1-6, or any combination thereof. Particularly, a training set comprises methylation pattern data from the methylation sites presented in Tables 1-6 In some embodiments, the methylation pattern data comprises data of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226; 227, 228, 229, 230, 231, 232, 233, 234, 235, 236, 237, 238, 239, 240, 241, 242, 243, 244, 245, 246, 247, 248, 249 or 250 methylation sites. In some embodiments, the methylation pattern data comprises data of more than 50 methylation sites. In some embodiments, the methylation pattern data comprises data of more than 100 methylation sites. In some embodiments, the methylation pattern data comprises data of more than 200 methylation sites. In some embodiments, the methylation pattern data comprises between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250 methylation sites selected from Tables 1-6.

In some embodiments, the training dataset comprises clinical variables for each subject, for example the subject classification according to a classification model disclosed herein. In other embodiments, the training data comprises data about the subject such as body weight, presence or absence of biomarkers, medication, etc.

In some embodiments, the training set includes a reference population of at least about: 5, 10, 20, 30, 40, 0, 60, 70, 80, 90, 100, 110, 120, 130, 140, 150, 160, 170, 180, 190, 200, 250, 300, 350, 400, 450, 500, 600, 700, 800, 900, or at least about 1000 subjects. In other embodiments, the training set includes more than 1000 subjects.

In some embodiments, the classification model comprises determining a (relative) weight for each methylation pattern and each clinical variable that is taken into account. In other embodiments, the classification model comprises determining a (relative) weight for each methylation pattern and/or each clinical variable. In other embodiments, the classification model comprises determining a weight for each methylation pattern, each clinical variable, and/or any additional variable.

In some embodiments, the classification model uses data indicating the (relative) weight of each methylation pattern and each clinical variable for the identification of DLB in a subject. In other embodiments, the classification model uses data indicating the (relative) weight of each methylation pattern and/or each clinical variable for the identification of DLB in a subject.

In some embodiments, determining the score comprises correlating each of at least one of the methylation patterns determined and each of at least one clinical variable with its determined weight. In some embodiments, determining the score comprises correlating each of at least one of the methylation patterns determined and/or each of at least one clinical variable with its determined weight.

In terms of variables regarding the mitochondrial methylation patterns of different sites, those variables contributing the most to identify DLB in a subject can differ. This is because the contribution of each variable depends on several aspects, the number of variables considered in the model (e.g., which clinical variables are included), the number of subjects used in the training of the classification model (in order to avoid over/under-fitting issues), the customization process of the parameters during the training process of the model, etc.

In those lines, the variables included in each classification model will depend on the information available from each subject, and consequently the importance/contribution of each selected variable will vary among classification models. Thus, it is difficult to define an exact set of variables or a specific number of variables to be considered when constructing a classification model. Contrariwise, it is desirable to develop classification models with the ability to adapt to the information available and can be constructed using variables selected according to their importance/contribution in each particular situation. In other words, the variables included in each classification model will be defined according to their contribution during the training process, rather than arbitrarily pre-defining a set of variables or a minimum number of variables that may not contribute as much in other scenarios. Whichever the case, the main objective is achieve an Accuracy value as higher as possible.

Classification models described herein can include different sets and combinations of methylation patterns and/or clinical variables. The classification model selects the methylation patterns and/or clinical variables according to the contribution or importance associated to each methylation pattern and/or clinical variable (i.e., determined weight). That is, the classification model is configured to combine the methylation pattern of sites which are correlated to a determined weight (e.g., 0.25, 0.5, 1, 2, 2.5, 5, etc.) and/or clinical variables which are correlated to a determined weight of at least 1 (e.g., 0.25, 0.5, 1, 2, 2.5, 5, etc.).

In some embodiments, the determined weight correlated to a methylation pattern or a clinical variable ranges or a combination of measured variables (e.g. principal components) from 0 to 100. Alternatively, the determined weight correlated to a methylation pattern, or a clinical variable is selected from 0 to 100. The value “0” corresponds to the lowest variable importance or lowest determined weight and the value “100” corresponds to the highest variable importance or highest determined weight. In a particular embodiment, the determined weight is selected from the group consisting of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

In some embodiments, the determined weight is at least 0.25. Particularly, the determined weight is selected from the group consisting of 0.25, 0.3, 0.35, 0.40, 0.45, 0.5, 0.55, 0.6, 0.65, 0.7, 0.75, 0.8, 0.85, 0.9, 0.95 and 1. In another embodiment, the determined weight is at least 1. Particularly, the determined weight is selected from the group consisting of 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 15, 20, 25, 30, 35, 40, 45, 50, 55, 60, 65, 70, 75, 80, 85, 90, 95 and 100, In another embodiment the determined weight is at least 2. Particularly, the determined weight is selected from the group consisting of 2, 2.25, 2.5, 2.75, 3, 3.25, 3.5, 3.75, 4, 4.25, 4.5, 4.75, 5, 6, 7, 8, 9, 10, 15, 20, 25, 30, 35, 40, 45, 50, 55, 60, 65, 70, 75, 80, 85, 90, 95 and 100. In another embodiment, the determined weight is selected from the group consisting of at least 1, at least 2, at least 2.5, at least 3, at least 3.5, at least 4, at least 4.5, at least 5, at least 7.5, at least 10, at least 15, at least 20, at least 25, at least 50 and at least 75.

In some embodiments, determining DLB in a subject comprises considering the methylation pattern of sites which are correlated to a determined weight of at least 0.5. In another embodiment, determining DLB in a subject comprises combining the methylation pattern of sites which are correlated to a determined weight of at least 0.5.

In another embodiment, determining DLB in a subject comprises considering the clinical variables which are correlated to a determined weight of at least 0.5. In another embodiment, determining DLB in a subject comprises combining the clinical variables which are correlated to a determined weight of at least 0.5.

In another embodiment, determining DLB in a subject comprises combining the methylation pattern of sites which are correlated to a determined weight of at least 0.5 with clinical variables which are correlated to a determined weight of at least 0.5. Alternatively, determining DLB in a subject comprises combining or considering methylation patterns and/or clinical variables correlated with a determined weight of at least 0.5.

In some embodiments, determining DLB in a subject comprises considering the methylation pattern of sites which are correlated to a determined weight of at least 1. In another embodiment, determining DLB in a subject comprises combining the methylation pattern of sites which are correlated to a determined weight of at least 1.

In another embodiment, determining DLB in a subject comprises considering the clinical variables which are correlated to a determined weight of at least 1. In another embodiment, determining DLB in a subject comprises combining the clinical variables which are correlated to a determined weight of at least 1.

In another embodiment, determining DLB in a subject comprises combining the methylation pattern of sites which are correlated to a determined weight of at least 1 with clinical variables which are correlated to a determined weight of at least 1. Alternatively, determining DLB in a subject comprises combining or considering methylation patterns and/or clinical variables correlated with a determined weight of at least 1.

In some embodiments, determining DLB in a subject comprises considering the methylation pattern of sites which are correlated to a determined weight of at least 2. In another embodiment, determining DLB in a subject comprises combining the methylation pattern of sites which are correlated to a determined weight of at least 2.

In another embodiment, determining DLB in a subject comprises considering the clinical variables which are correlated to a determined weight of at least 2. In another embodiment, determining DLB in a subject comprises combining the clinical variables which are correlated to a determined weight of at least 2.

In another embodiment, determining DLB in a subject comprises combining the methylation pattern of sites which are correlated to a determined weight of at least 2 with clinical variables which are correlated to a determined weight of at least 2. Alternatively, determining DLB in a subject comprises combining or considering methylation patterns and/or clinical variables correlated with a determined weight of at least 2.5.

In some embodiments, determining DLB in a subject comprises considering the methylation pattern of sites which are correlated to a determined weight of at least 2.5. In another embodiment, determining DLB in a subject comprises combining the methylation pattern of sites which are correlated to a determined weight of at least 2.5.

In another embodiment, determining DLB in a subject comprises considering the clinical variables which are correlated to a determined weight of at least 2.5. In another embodiment, determining DLB in a subject comprises combining the clinical variables which are correlated to a determined weight of at least 2.5.

In another embodiment, determining DLB in a subject comprises combining the methylation pattern of sites which are correlated to a determined weight of at least 2.5 with clinical variables which are correlated to a determined weight of at least 2.5. Alternatively, determining DLB in a subject comprises combining or considering methylation patterns and/or clinical variables correlated with a determined weight of at least 2.5.

In some embodiments, determining DLB in a subject comprises considering the methylation pattern of sites which are correlated to a determined weight of at least 5. In another embodiment, determining DLB in a subject comprises combining the methylation pattern of sites which are correlated to a determined weight of at least 5.

In another embodiment, determining DLB in a subject comprises considering the clinical variables which are correlated to a determined weight of at least 5. In another embodiment, determining DLB in a subject comprises combining the clinical variables which are correlated to a determined weight of at least 5.

In another embodiment, determining DLB in a subject comprises combining the methylation pattern of sites which are correlated to a determined weight of at least 5 with clinical variables which are correlated to a determined weight of at least 5. Alternatively, determining DLB in a subject comprises combining or considering methylation patterns and/or clinical variables correlated with a determined weight of at least 5.

Alternatively, the methylation patterns and/or clinical variables to be combined or considered by the classification model for determining DLB in a subject are selected according to their determined weight. Particularly, the methylation patterns and/or clinical variables combined by the classification model are selected according to their determined weight and the determined weight is at least 1. In another particular embodiment, the methylation patterns and/or clinical variables combined by the classification model are selected according to their determined weight and the determined weight is at least 2. In some embodiments, the methylation patterns and/or the clinical variables combined by the classification model for determining DLB in a subject are correlated to a determined weight selected from the group consisting of at least 0.25, at least 0.5, at least 1, at least 2, at least 2.5, at least 3, at least 3.5, at least 4, at least 4.5, at least 5, at least 7.5, at least 10, at least 15, at least 20, at least 25, at least 50 and at least 75.

In some embodiments, the classification model is capable of classifying subjects into two categories: CTL or DLB. In some embodiments, the classification model calculates a risk score to each category, corresponding to the probability of the subject to be assigned each category. In some embodiments, the probabilities to each category add up to 1.

In some embodiments, the classification model is capable of classifying subjects into two categories: subjects with DLB and control subjects. In some embodiments, the classification model calculates a score to each category, corresponding to the probability of the subject to be assigned each category. In some embodiments, the probabilities to each category add up to 1.

Hereinbefore, weights correlated to a methylation pattern or a clinical variable range from 0 to 100 or are selected from 0 to 100, i.e. a scale of 0 to 100 is used. For this scale, specific minimum values of weights have been defined herein. It should be clear however, that any other suitable scale can be used, e.g., a scale from 0 to 1 or a scale from 0 to 1000 or a scale from 0 to 20. With such a different scale, the minimum values of weights mentioned hereinbefore can be changed proportionally.

The classification models generated by the machine learning methods disclosed herein or any other supervised method for classification known in the art (e.g., LDA, CART, KNN, NB, SVM with a linear kernel, RF, NNET, GBM and GLM) can be subsequently evaluated by determining the ability of said model (i.e., classifier) to correctly classify each test subject. In some embodiments, the subjects of the training population used to derive the model are different from the subjects of the testing population used to test the model. As would be understood by a person skilled in the art, this allows one to predict the ability of the dataset used to train the classifier as to their ability to properly classify a subject whose output classification (e.g., dementia stage classification, i.e., subjects with DLB and control subjects) is unknown.

In some embodiments, the classification model is evaluated for its ability to properly classify each subject of the training population using methods known to a person skilled in the art. For example, one can evaluate the classification model using Cross Validation (CV), Leave One Out Cross Validation (LOOCV), k-fold cross validation, or Jackknife analysis using standard statistical methods. In other embodiments, each classifier is evaluated for its ability to properly characterize those subjects of the training population which were not used to generate the classifier.

In some embodiments, the metrics used to evaluate the classification model for its ability to properly classify each subject of the training population are selected from the group consisting of classification accuracy (ACC), Kappa statistic, Error Rate (i.e., misclassification rate), Sensitivity (True Positive Fraction or Rate, TPF or TPR), Specificity (True Negative Fraction or Rate, TNF or TNR), Positive Predicted Value (PPV), Negative Predicted Value (NPV) or any combination thereof. In some embodiments, the metrics used to evaluate the classification model for its ability to properly classify each subject of the training population further comprise Recall, Precision, F-score (F-measure or F1 score), confusion matrix (or contingency table) and/or Area Under the Receiver Operating Characteristic Curve (AUC ROC).

In a particular embodiment, the metrics used to evaluate the classification model for its ability to properly classify each subject of the training population comprises evaluating model's sensitivity (TPF, true positive fraction) and 1-specificity (FPF, false positive fraction). In one embodiment, the method used to test the classifier is Receiver Operating Characteristic (“ROC”), which provides several parameters to evaluate both the sensitivity and specificity of the result of the classification model generated.

Another aspect of the invention relates to a computer-implemented method for the identification of DLB in a subject, comprising
(a) providing or receiving data relating to the methylation pattern in the D-loop region and/or the ND1 gene of the mitochondrial DNA of a subject, wherein the methylation pattern is determined in at least one site selected from the group consisting of:
(i) the CpG sites in the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6, and optionally at least one clinical variable of the subject, and
(b) determining a risk score correlating to the identification of DLB in a subject, wherein the risk score is calculated or determined using a classification model configured to combine the methylation pattern of one or more sites of step (a) and optionally at least one clinical variable of the subject.

Another aspect of the invention relates to a computer-implemented method for obtaining a score which correlates to the identification of DLB in a subject, the method comprising the following steps:
(a) providing or receiving as input data:
(1) the methylation pattern in the D-loop region and/or the ND1 gene of the mitochondrial DNA of a subject, wherein the methylation pattern is determined in at least one site selected from the group consisting of:
(i) the CpG sites in the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2,
(v) the CHG sites of the ND1 gene shown in Table 4, and
(vi) the CHH sites of the ND1 gene shown in Table 6, and optionally
(b) weighting said methylation patterns using a classification model to obtain the score.

Another aspect of the invention relates to a computer-implemented method for obtaining a score which correlates to the identification of DLB in a subject, the method comprising the following steps:
(a) providing or receiving as input data:
(1) at least one clinical variable of the subject, and
(b) weighting said clinical variables using a classification model to obtain the score.

In some examples, input data can be received from which a computer or other data processing system can derive the methylation pattern in the D-loop region and/or the methylation pattern in the ND1 gene of the mitochondrial DNA of a subject. And the computer-implemented method can then further comprise receiving at least one clinical variable of the subject as described herein, and combining and weighting the methylation pattern(s) and clinical variable(s) using a classification model to obtain a risk score.

### Score

In some embodiments, the score is a score of 0-1, where 0 indicates lowest probability of having DLB and 1 indicates highest probability of having DLB in the subject. In a particular embodiment, a score of 0.5 or higher than 0.5 indicates that the subject is likely to have DLB. Alternatively, a score of 0.5 or higher than 0.5 indicates that the subject has a high probability of having DLB. In a particular embodiment, a score of 0.75 or higher than 0.75 indicates that the subject has a very high probability of having DLB. In some embodiments, a score lower than 0.5 indicates that the subject has a low probability of having DLB. In a particular embodiment, a risk score of 0.25 or lower than 0.25 indicates that the subject has very low probability of having DLB.

In some embodiments, the score is a score of 0-100, where 0 indicates lowest probability of the subject of having DLB and 100 indicates highest probability of the subject of having DLB. In some embodiments, the score can be but is not limited to 1-2, 1-5, 1-10, 1-100, 0-10 and 0-100. It is understood that the score in the present invention can be any range of values which serve to correlate to the probability of a subject of having DLB.

### Companion Diagnostic Systems

The methods disclosed herein can be provided as a companion diagnostic, e.g., available via a web server, to inform the clinician of patient about potential treatment choices or for the selection of patients for a clinical trial. The methods disclosed herein can comprise collecting or otherwise obtaining a biological sample and performing an analytical method disclosed herein to diagnose DLB in a subject.

In an aspect of the present invention, a computing system is provided which comprises suitable means for carrying out any of the computer-implemented methods described herein.

At least some embodiments of the methods described herein, due to the complexity of the calculations involved can be implemented with the use of a computer. In some embodiments the computer system comprises hardware elements that are electrically coupled via bus, including a processor, input device, output device, storage device, computer-readable storage media reader, communications system, processing acceleration (e.g., DSP or special-purpose processors), and memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that embodiments can well be utilized in accordance with more specific application requirements. Customized hardware might also be utilized and/or particular elements might be implemented in hardware, software, firmware or combinations thereof. Further, while connection to other computing devices such as network input/output devices (not shown) can be employed, it is to be understood that wired, wireless, modem, and/or other connection or connections to other computing devices might also be utilized.

In one embodiment, the system further comprises one or more devices for providing input data to the one or more processors. The system further comprises a memory for storing a dataset of ranked data elements. In another embodiment, the device for providing input data comprises a detector for detecting the characteristic of the data element, e.g., such as a fluorescent plate reader, mass spectrometer, or gene chip reader.

The system can additionally comprise a database management system. User requests or queries can be formatted in an appropriate language understood by the database management system that processes the query to extract the relevant information from the database of training sets. The system can be connectable to a network to which a network server and one or more clients are connected. The network can be a local area network (LAN) or a wide area network (WAN), as is known in the art. Particularly, the server includes the hardware necessary for running computer program products (e.g., software) to access database data for processing user requests. The system can be in communication with an input device for providing data regarding data elements to the system (e.g., methylation patterns).

In a further aspect, the invention is directed to a computer program product comprising instructions which, when the program is executed by a computer, cause the computer to carry out any of the computer-implemented methods described herein.

Some embodiments described herein can be implemented so as to include a computer program product. A computer program product can include a computer readable medium having computer readable program code embodied in the medium for causing an application program to execute on a computer with a database. As used herein, a “computer program product” refers to an organized set of instructions in the form of natural or programming language statements that are contained on a physical media ofany nature (e.g., written, electronic, magnetic, optical or otherwise) and that can be used with a computer or other automated data processing system. Such programming language statements, when executed by a computer or data processing system, cause the computer or data processing system to act in accordance with the particular content of the statements.

Computer program products include without limitation; programs in source and object code and/or test or data libraries embedded in a computer readable medium. Furthermore, the computer program product that enables a computer system or data processing equipment device to act in pre-selected ways can be provided in a number of forms, including, but not limited to, original source code, assembly code, object code, machine language, encrypted or compressed versions of the foregoing and any and all equivalents. In one aspect, a computer program product is provided to implement the treatment, diagnostic, methods disclosed herein, for example, to determine whether to administer a certain therapy based on the obtained score.

The computer program product includes a computer readable medium embodying program code executable by a processor of a computing device or system, the program code comprising:
(a) code that retrieves data attributed to a biological sample from a subject, wherein the data comprises the methylation patterns corresponding to at least one methylation site of methylation sites of Tables 16 in the biological sample or wherein the methylation patterns can be derived from the data. These values are combined with values corresponding to clinical variables; and,
(b) code that executes a classification method that indicates, e.g., whether to administer a therapeutic agent to a patient in need thereof based on the obtained scores.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description.

Furthermore, some embodiments can be code stored in a computer-readable memory of virtually any kind including, without limitation, RAM, ROM, magnetic media, optical media, or magneto-optical media.

Even more generally, some embodiments could be implemented in software, or in hardware, or any combination thereof including, but not limited to, software running on a general-purpose processor, microcode, PLAs, or ASICs.

It is also envisioned that some embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### Performance of the Methods

In some embodiments, samples can, e.g., be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, determining the methylation patterns disclosed herein; obtaining clinical variables from a subject; combining the methylation patterns with clinical information; identifying/diagnosing DLB in a subject, the application of the classification model; the determination of scores; diagnostic/prognostic decisions; treatment decisions; clinical trials inclusion decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, diagnosis, assessment, maintenance, therapy, medication, and/or advice relating to all, or any portion of, a patient's state of health, including but not limited to general medical, specialized medical, surgical, and/or any other type of treatment, diagnosis, assessment, maintenance, therapy, medication and/or advice. A healthcare provider also refers herein to pharmaceutical companies or its providers/intermediates (e.g., CRO) involved in the development of clinical trials.

As used herein, the term “clinical laboratory” refers to a facility for the examination or processing of materials derived from a subject. These examinations can also include procedures to collect or otherwise obtain a sample, prepare, determine, measure, or otherwise describe the presence or absence of various substances in the body of a subject or a sample obtained from the body of a subject (e.g., methylation patterns of mtDNA or biomarkers used herein as clinical variables) Examinations can also include procedures such as medical imaging procedures (e.g., PET, MRI) to obtain clinical variables data.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

A healthcare provider can implement or instruct another healthcare provider or patient to perform e.g., the following actions: obtain a sample/clinical variables, process a sample/clinical variables, submit a sample/clinical variables, receive a sample/clinical variables, transfer a sample/clinical variables, analyze or measure a sample/clinical variables (e.g. to obtain the methylation patterns), quantify a sample/clinical variables, provide the results obtained after analyzing/measuring/quantifying a sample/clinical variables, receive the results obtained after analyzing/measuring/quantifying a sample/clinical variables, apply the classification model, obtain the results after analyzing/measuring/quantifying one or more samples/clinical variables, provide the results, diagnose/identify DLB in a subject, identify a subject suitable for treatment of DLB, select a subject for submission to treatment for DLB, select a suitable therapy to treat a subject with DLB, classify a subject according to the presence or absence of DLB; treat DLB in a subject comprising administering a treatment for DLB if the subject has been identified with DLB, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent, and/or monitor the progression of DLB.

In some embodiments, a healthcare benefits provider can authorize or deny any action described above, e.g., collection of a sample/clinical variables, processing of a sample/clinical variables, submission of a sample/clinical variables, receipt of a sample/clinical variables, transfer of a sample/clinical variables, analysis or measurement a sample/clinical variables (e.g. to obtain the methylation patterns), quantification of a sample/clinical variables, apply the classification model, provision of results obtained after analyzing/measuring/quantifying a sample/clinical variables, transfer of results obtained after analyzing/measuring/quantifying a sample/clinical variables, scoring of results obtained after analyzing/measuring/quantifying one or more samples/clinical variables, transfer of the results from one or more samples/clinical variables, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent. In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some embodiments, a clinical laboratory can, e.g., collect or obtain a sample/clinical variables, process a sample/clinical variables, submit a sample/clinical variables, receive a sample/clinical variables, transfer a sample/clinical variables, analyze or measure a sample/clinical variables (e.g. to obtain the methylation patterns), quantify a sample/clinical variables, apply the classification model, provide the results obtained after analyzing/measuring/quantifying a sample/clinical variables, receive the results obtained after analyzing/measuring/quantifying a sample/clinical variables, obtain the results after analyzing/measuring/quantifying one or more samples/clinical variables, provide the results, diagnose/identify DLB in a subject, identify a subject suitable for treatment of DLB, select a subject for submission to treatment for DLB, classify a subject according to the presence or absence of DLB, monitor the progression of DLB, or other related activities.

In some embodiments, the sample/clinical variables can be obtained by a healthcare professional treating or diagnosing the patient, by a healthcare provider or by a clinical laboratory. Measurements of the sample (e.g., by using a particular assay described herein) and obtaining clinical variables (e.g., by medical imaging techniques) can be performed by a healthcare provider or a clinical laboratory, being the same or different from the ones that obtained the sample/clinical variables. The classification model can be applied by the healthcare provider or a different healthcare provider or by the clinical laboratory. The results obtained are finally sent to the first healthcare professional treating or diagnosing the patient or to the healthcare provider: Thus, in some embodiments, the healthcare provider or the clinical laboratory can advise the healthcare professional/provider about diagnosis/prognosis or as to whether the patient can benefit from treatment. In some embodiments, the healthcare provider is a pharmaceutical company or one of its providers/intermediates (e.g., CRO) involved in the development of clinical trials. All the steps described herein can be performed by the pharmaceutical company and/or one of its providers/intermediates or performed in part e.g., by a clinical laboratory or a different healthcare provider.

### EXAMPLES

### Example 1: Detection of mtDNA Methylation in Blood Samples

### 1.1 Materials and Methods

### 1) Blood Samples Collection

Human blood samples were collected in EDTA tubes to prevent blood coagulation. After obtention of samples in the laboratory, blood was directly processed for DNA extraction or aliquoted and stored at 80° C. until processed.

Samples from a total of 36 subjects were extracted, which were recruited from three different cohorts. Controls have been recruited from two different cohorts: Cohort A corresponds to the Australian Imaging, Biomarker & Lifestyle Flagship Study of Ageing (AIBL) and Cohort B corresponds to FundaciOn CITA (Centro de Investigacian y Terapias Avanzadas) in San Sebastian, Spain. Further, Cohort C corresponds to DLB patients recruited between 2015-2019 in Hospital de Bellvitge, Barcelona. Overall, subjects were classified in two groups: controls (50%, N=18) and DLB subjects (50%, N=18).

### 2) Total DNA Extraction

Whole blood samples were processed to achieve total DNA extraction and copurification of both the genomic and mitochondrial DNA. DNA was isolated from human whole blood samples using the Wizard® Genomic DNA Purification Kit (#A1620, Promega); according to the manufacturer's instructions. Alternatively, samples were processed with the Maxwell® RSC Instrument that provided an easy method for efficient, automated purification of DNA from samples. DNA sample capture, washing and purification was done using paramagnetic beads. The Maxwell® RSC Blood DNA Kit (#AS 1400, Promega) was used following manufacturer's specifications. The quality and quantity of purified DNA was determined with NanoDrop™ One Spectrophotometer from Thermofisher Scientific.

### 3) Bisulfite Treatment

Bisulfite conversion consists in the deamination of unmodified cytosines to uracil, leaving intact the modified bases 5-mC, i.e., methylated cytosines. Samples of total DNA (300 ng) were treated with bisulfite reagent using EZ DNA Methylation Kit (#D5001, Zymo Research), according to the manufacturer's protocol. In order to obtain a better bisulfite conversion, the incubation conditions of the step 2 of the protocol, which consisted in 15 min of incubation at 37° C., were substituted for 30 min of incubation at 42° C., as indicated in Appendix 1.A of the manufacturer's protocol. These last conditions are recommended to minimize an incomplete C to T Conversion. The treated DNA was finally resuspended in 30 pL of nuclease free water.

### 4) Amplicon Library Preparation

The workflow for amplicon library construction was based on the Illumina “16S Metagenomic Sequencing Library Preparation Protocol”, which can be used to sequence regions of the 16S rRNA gene and other targeted amplicon sequences of interest. The amplicon library preparation allowed the obtention of mtDNA amplicons of interest and their preparation for Illumina MiSeq System processing.

### 4.1) First PCR: Amplicon PCR

The mtDNA regions of interest were amplified by PCR with specific degenerated primers (see Section 1.2.1 of the results), corresponding to sequences SEQ ID NO: 1-4, further containing overhanged Illumina adapters. When designing the primers for the region of interest, an overhang adapter sequence had to be added to the locus-specific primer for the region to be targeted, as indicated by Illumina's protocol.

The Illumina® overhang adapter sequences to be added to locus-specific sequences are (SEQ ID NO: 5, 6):

Forward overhang:
5′TCGTCGGCAGCGTCAGATGTGTATAAGAGACAG-
[locus-specific sequence]
Reverse overhang:
5′GTCTCGTGGGCTCGGAGSTGTGTSTAAGAGACAG-
[locus-specific sequence]

Amplification of the bisulfite converted DNA was performed using The FastStart™ High Fidelity PCR System (#3553400001, Roche). The final PCR mixture (25 μL) contained: 5 μL of Bisulfited DNA, 1×FastStart Buffer #2; 0.05 U FastStart HiFi Polymerase; 0.8 mM total dNTP (0.2 mM each dNTP); and 0.4 μM each forward and reverse primers. The reaction for the ND1 amplicon also included 5% of DMSO. Final volume was adjusted with nuclease free water.

Amplifications were performed in a SimpliAmp™ Thermal Cyder from Applied Biosystems:

In order to evaluate the resulting products of the first PCR, 3 μL of each PCR product were analyzed by electrophoresis on 1.5% agarose gel stained with SybrSafe™ DNA Gel Stain (#S33102, Thermofisher Scientific) to verify the presence of the bands from the amplicons.

### 4.2) Clean-Up of the First PCR

AMPure XP beads (#A63881, Beckman Coulter) were used to purify the amplicons and separate them from free primers and primer dimer species. Steps were performed according to Illumina “16S Metagenomic Sequencing Library Preparation Protocol”. A ratio of 0.8× of AMpure Beads was used to purify the PCR amplicon product. Bead elution was performed in 14 μL of Buffer EB (#19086, Qiagen) and 12 μL were recovered from the beads.

### 4.3) Second PCR: Index PCR

Index PCR was performed to attach unique dual indices (UDI) and sequencing adapters from Illumina. PCR Index reaction was performed in a 50 μL reaction containing: 5 μl of DNA purified in the first PCR Clean-Up, 25 μL of KAPA HiFi HotStart Ready Mix (2×) (#7958935001, Roche), 10 μL of Unic dual indexes from Illumina, and 10 μL of Nuclease free water. It was performed using a SimpliAmp™ Thermal Cyder from Applied Biosystems™.

### 4.4) Clean-Up of the Second PCR

Second PCR Clean-Up was performed using AMPure XP beads to clean up the final library before quantification. The 50 μl of the second PCR reaction were purified following the steps described in “16S Metagenomic Sequencing Library Preparation Protocol” from Illumina. A ratio of 1.12× of AMPure XP beads was used, and a final elution was performed in 27.5 μL of Buffer EB and 25 μL were recovered from the beads.

### 4.5) Library Quantification, Normalization and Pooling

Quantification of the libraries was performed using a fluorometric quantification method that used dsDNA binding dyes with a Qubit® 3.0 Fluorometer, from Thermo Fisher Scientific. Quantification was performed with the Qubit™ dsDNA HS Assay Kit following manufacturer's instructions. After obtaining the Qubit quantification values in ng/μL, DNA concentration was calculated in nM, based on the size of DNA amplicons as determined by an Agilent Technologies 2100 Bioanalyzer trace with the following formula:

$$( Concentration in ng / μL ) / ( 660 g / mol \times average library size ) \times {10}^{6} = concentration in nM$$

To perform normalization, the final libraries were diluted using Buffer EB to 10 nM and a final 4 nM pool of the amplicon libraries was prepared with Buffer EB in 20 μL of final volume.

In parallel, from a 10 nM PhiX library (#FC-110-3001, Illumina), a 4 nM PhiX library dilution was prepared with Buffer EB in a 5 μL of final volume (each run had to include a minimum of 5% PhiX to serve as an internal control for these low-diversity libraries).

For the preparation of cluster generation and sequencing, pooled amplicon libraries were denatured with NaOH and diluted with HT1 Buffer as follows: 5 pL of the 4 nM amplicon library and 5 μL of 0.2 N NaOH (freshly prepared) were introduced in a microcentrifuge tube, mixed briefly using a vortex, and centrifuged at 280×g at 20° C. for 1 minute. An incubation of 5 minutes at room temperature was performed to denature the DNA, and 990 μL of pre-chilled HT1 Buffer were added to the 10 μL of denatured DNA. Finally, HT1 results were added in a 20 pM denatured amplicon library in 1 mM NaOH. The denatured DNA was placed on ice until proceeding to final dilution.

The same steps were repeated for the 5 pL of 4 nM PhiX library to denature and dilute PhiX to result in a 20 pM PhiX denatured library.

A 7 pM denatured amplicon library was prepared mixing 210 μL of the 20 pM denatured amplicon library with 390 μl of pre-chilled HT1 Buffer in a final volume of 600 μL. Afterwards, a 10 pM denatured PhiX library was prepared mixing 300 μL of the 20 pM denatured PhiX library with 300 μl of pre-chilled HT1 Buffer in a final volume of 600 μL.

At the end, 25% of denatured PhiX library and 75% of denatured amplicon library were combined into a final volume of 600 μL (150 μL from the 7 pM denatured amplicon library were discarded and were substituted by 150 μL of the 10 pM denatured PhiX library).

The combined amplicon library and PhiX control were set aside on ice until being ready to heat denature. The heat denaturation step was performed immediately before loading the library into the MiSeq reagent cartridge to ensure efficient template loading on the MiSeq flow cell.

Using a heat block, the combined library and PhiX control tube were incubated at 96° C. for 2 minutes. After the incubation, the tube was inverted 1-2 times to mix and immediately placed on ice. The tube was kept on ice for 5 minutes.

### 5) Template Loading and Run Setting on MiSeq Instrument

The Sequencing on MiSeq Instrument Using paired 300-bp reads was prepared, along with the MiSeq reagent Kit v3 (#MS-102-3003, Illumina).

When the Illumina v3 reagent cartridge was fully thawed and ready for use, prepared libraries were loaded onto the cartridge and run was set on MiSeq instrument following manufacturer's instructions.

### 6) Calculation of Mitochondrial Methylation Percentages

The percentage (%) of methylation of each cytosine site is calculated by means of the beta value (β). The β value is the ratio of methylated reads per site and the overall sum of methylated and unmethylated reads per site; that is:

$${β}_{i} = M / ( M + U )$$

where M is the number of methylated reads in site (i) and U is the number of unmethylated reads in the same site (i).
β values are between 0 and 1 with 0 being completely unmethylated and 1 being fully methylated.

The percent of methylation (%) of each site is obtained by multiplying *100 the β value; that is:

$${\%}_{i} = {β}_{i} * 100$$

### 7) Differential Methylation Analysis

In order to identify the differentially methylated sites (DMS, also referring to DML for differentially methylated loci), an standard pipeline consisting of the following five main steps was carried out:
1. Quality control of raw data: quality assessment of the reads generated with Illumina equipment during the sequencing process.
2. Raw data pre-processing: preparation of data for the alignment process by filtering, clipping and trimming raw reads according to different criteria.
3. Alignment process: mapping pre processed reads to the reference genome.
4. Methylation calling (quantification): Identification of cytosine positions for each context (i.e., CpG, CHG and CHH) and the number of methylated and unmethylated reads per each site, region and sample.
5. Pile-up methylation profiles: Compilation and building of methylation, unmethylation and percentage matrices to be analyzed (where rows are samples and columns are the identified positions) and execution of a quality control of the measures reported.

Based on the mitochondrial methylation percentage matrices (referred to as methylation measures) two data analyses were conducted: (1) Exploratory Data Analysis, and (2) identification of DMS
1) Exploratory data analysis (EDA) was intended to describe the distribution of the measures at both sample and position levels for each phenotype group, context and region. To this end, two different plots are reported. The Boxplot of data (also referred to as box plot, box-and-whisker plot or box-and-whisker diagram) graphically depicts the distribution of the percentage of methylation through their quartiles for different samples in each position in the comparison of DLB vs CTL samples in each context. CTL samples are represented in black and DLB samples in gray. In the boxplot, the middle box is the inter-quartile range and represents the middle 50% of data. In each box is represented the median (middle quartile) that marks the mid-point of the data and is shown by the line that divides the box into two parts. The lower whiskers represent the quartile 25 and the upper whisker the quartile 75, at the end whiskers the maximum and minimum are represented. A global median is also represented by a horizontal gray line and is the global median for all existing values in each group. Further, a global mean is also represented in the CTL group and the DLB group by a dashed horizontal gray line that shows the overall mean of methylation percentages. The second type of graphic, Mean and confidence interval (95%) graphic represents the mean of the percentage of methylation of all positions and its confidence interval (95%) for each subject in CTL and DLB. The mean is represented by a black circle in CTL and with a grey circle in DLB and the confidence intervals are represented by a vertical bar. A global median is also represented in CTL group and DLB group by a horizontal gray line that shows the global median for all existing values in each group of samples. A global mean is also represented in the CTL group and the DLB group by a dashed horizontal gray line that shows the overall mean of methylation percentages.
2) Identification of Differentially Methylated Sites (DMS): The analysis to compare methylation levels between groups (i.e., subjects with DLB and controls) in each methylation site was conducted using DSS (Dispersion Shrinkage for Sequencing data) Bioconductor package. This package is intended to identify Differentially Methylated Loci/Sites (DML/DMS) on bisulfite sequencing (BS-seq) data. The core of DSS is a procedure based on Bayesian hierarchical model to estimate and shrink cytosine site-specific context dispersions, and then conducting Wald tests for beta-binomial distribution to detect differential methylation. Moreover, for general experimental design, DSS is based on a beta-binomial regression model considering an arcsine link function, and the model fitting is conducted on transformed data with generalized least quare method.

Multiple testing problem was addressed by adjusting the Benjamini-Hochberg False Discovery Rate (FDR), i.e., p-values were adjusted using FDR methods. Other methods can be used to adjust p-values, such as Family Wise Error Rate (FWER). Cytosine site-specific contexts with an adjusted p-value (i.e., FDR) smaller than 0.05 were identified as differentially methylated. The model is set with one single main effect. Furthermore, in case of having technical biases due to batch effects or some sort of technical variability, these unwanted effects are considered in the model.

### 1.2 Results

1.2.1 Optimization of mtDNA Methylation Detection

The primers used herein (mentioned in section 4-Amplicon library preparation) were designed for an optimized detection of mtDNA methylation. In order to avoid a bias derived from the general assumption in the art that non-CpG cytosines are mainly unmethylated, inventors used primers which included the least number of cytosines. Furthermore, the primers were degenerated to cover all possible methylated and no methylated scenarios, thus addressing the problem resulting from the uncertainty of the C/U conversion, which could affect the few cytosines residues included in the sequences. Said degeneration consisted in the inclusion of a mixture of oligonucleotide sequences which overall contained several possible nucleotide bases at each specific position, therefore significantly increasing the probability to detect mitochondrial methylation.

In summary, the forward degenerated primers include Y, which refers to either C or T (Y=C/T), in any position wherein the reference sequence is a C. Further, as known in the art, reverse primers do not correspond to the reference sequence, but to the reversed complementary of the reference sequence. Therefore, the reverse primers do not include C sites of the reference sequence, but their complementary G sites. Consistently, reverse degenerated primers include R, which refers to either G or A (R=A/G), in any position wherein the reference sequence is a C, or wherein the complementary sequence includes a G. As a result, the four degenerated primers are:

### D-Loop Region:

Forward Primer:
(SEQ ID NO: 1)
YAYTTGGGGGTAGYTAAAGTGAAYTG
Reverse primer:
(SEQ ID NO: 2)
TCCTACAARCATTAATTAATTAACACAC
ND1 gene;
(SEQ ID NO: 3)
Forward Primer:
ATAAAAYTTAAAAYTTTAYAGTYAGAG
Reverse primer:
(SEQ ID NO: 4)
TTRARTTTRATRCTCACCCTRATCA

### 1.2.2 Mitochondrial DNA Methylation Patterns

Methylation levels were compared between DLB subjects (N=18) and control subjects (N=18) for two different regions, i.e., ND1 gene and the D-loop region in three different contexts: CpG, CHG and CHH.

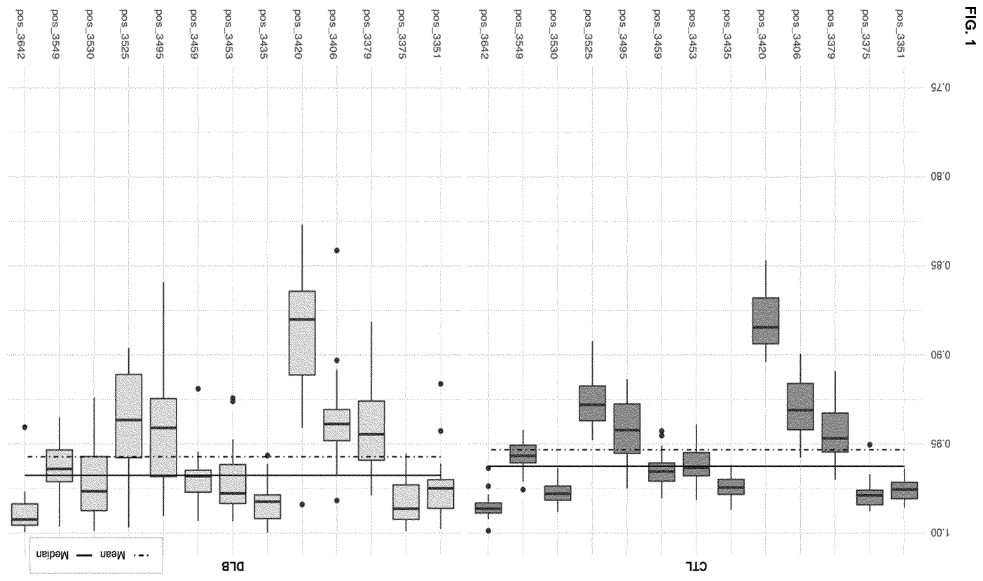

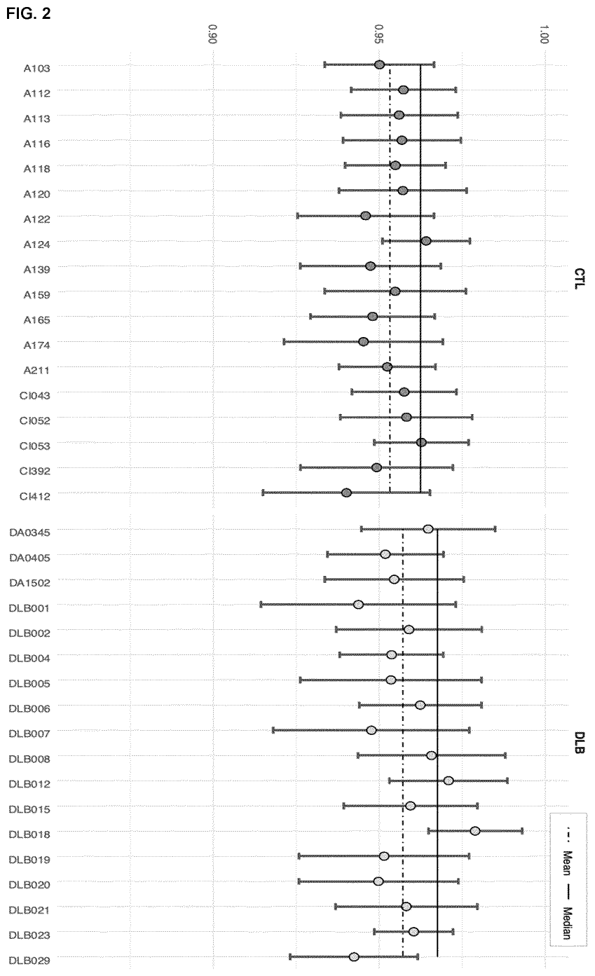

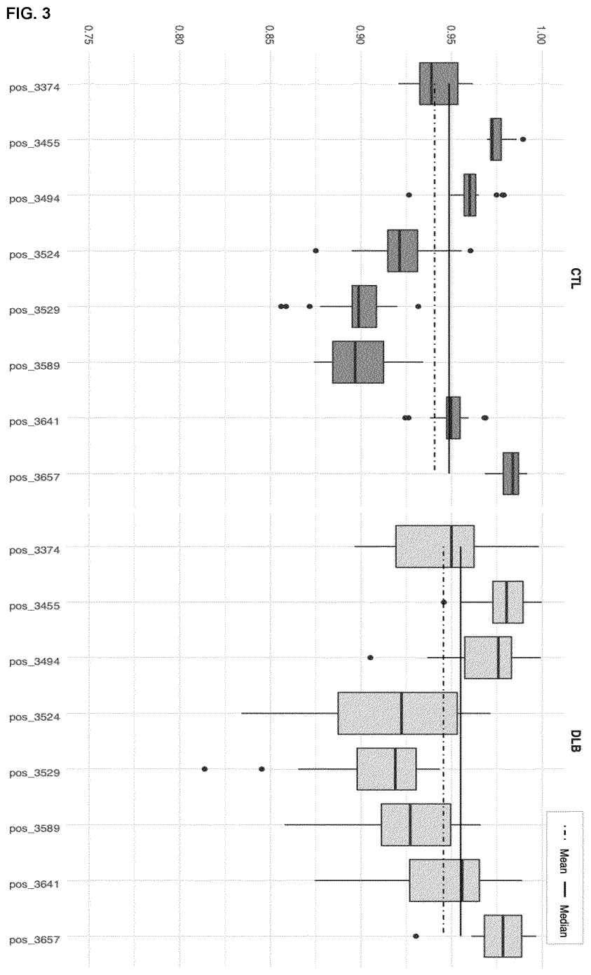

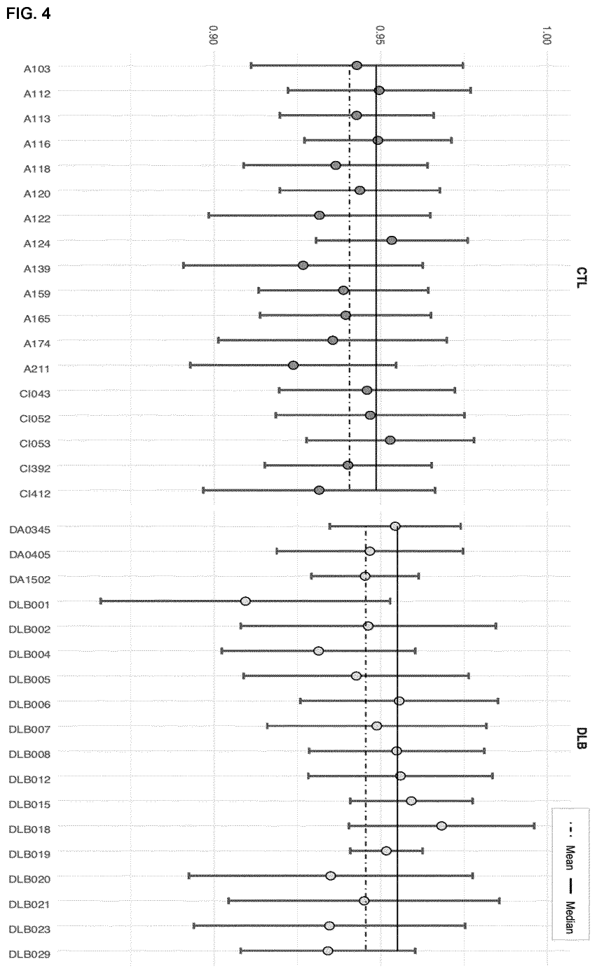

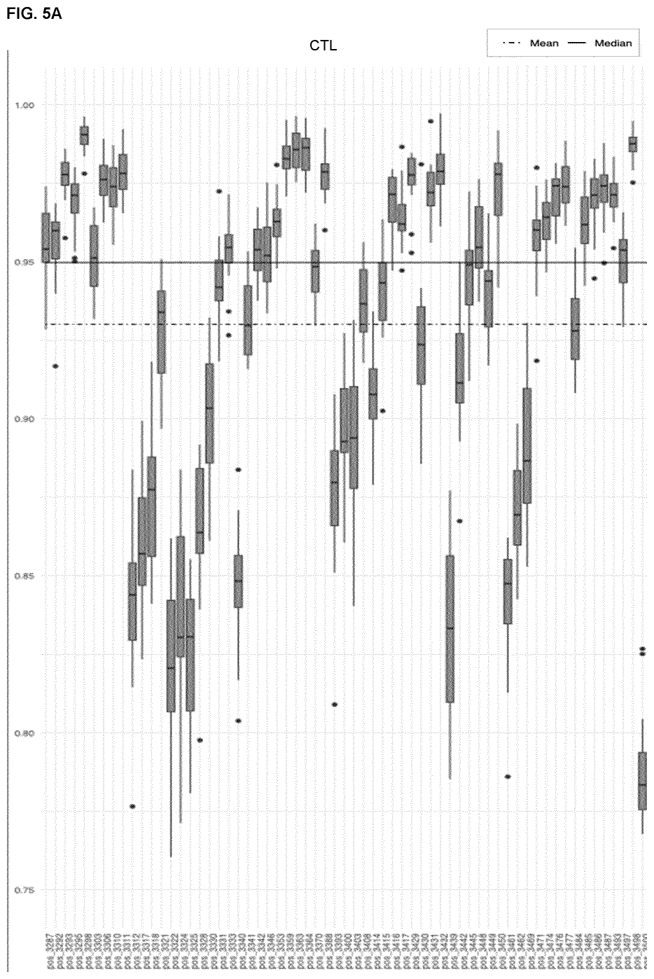

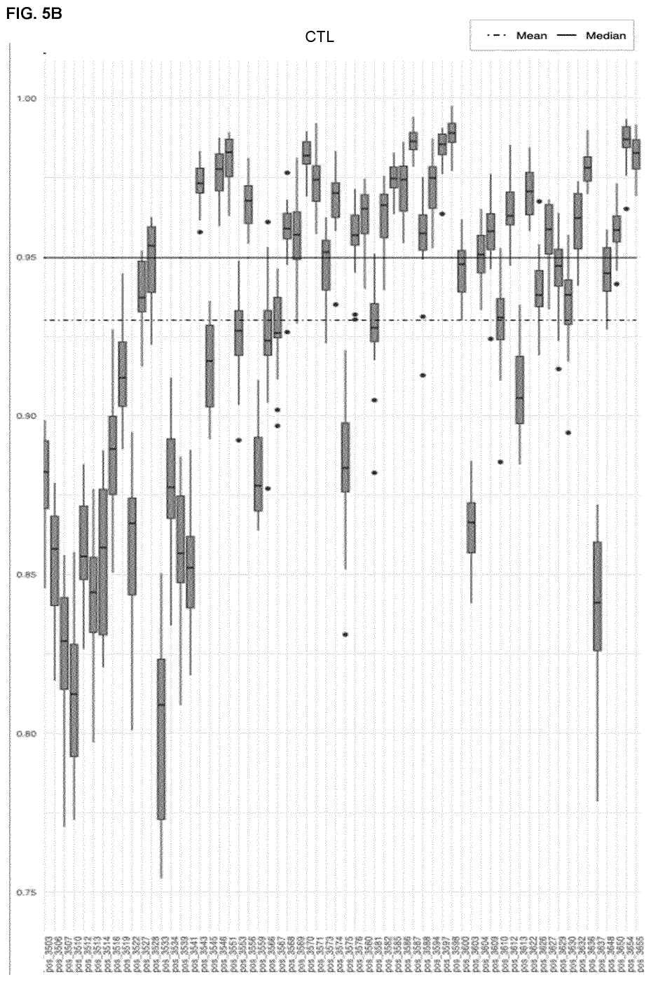

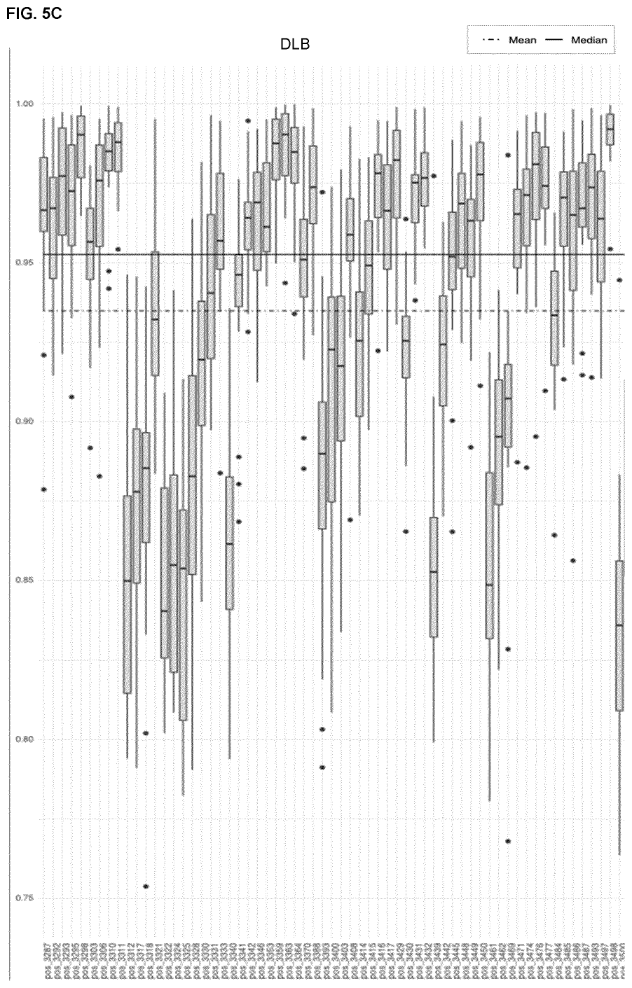

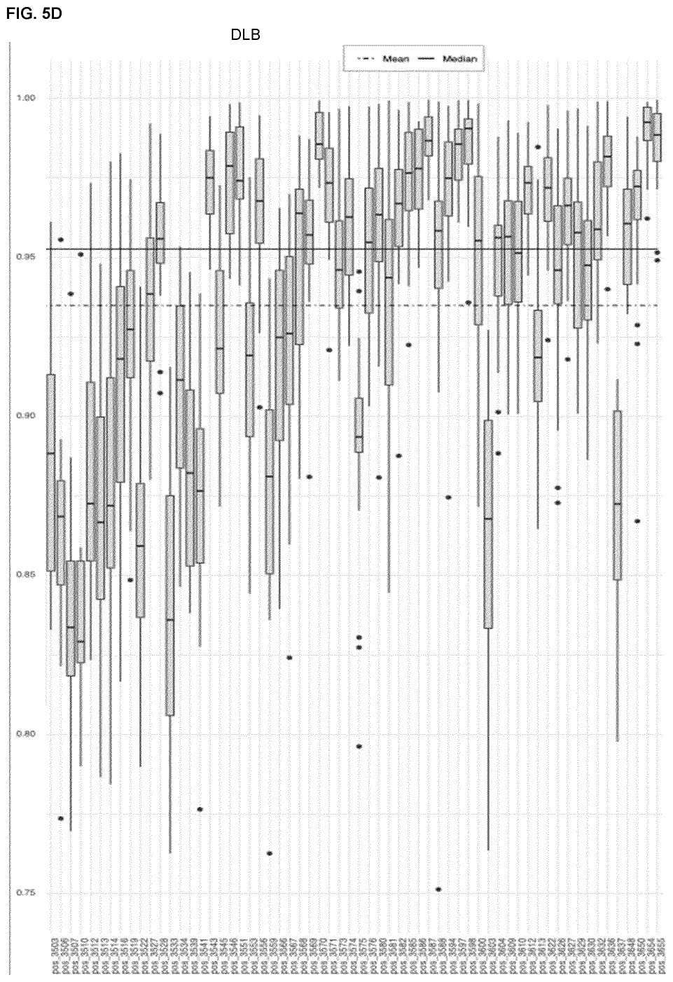

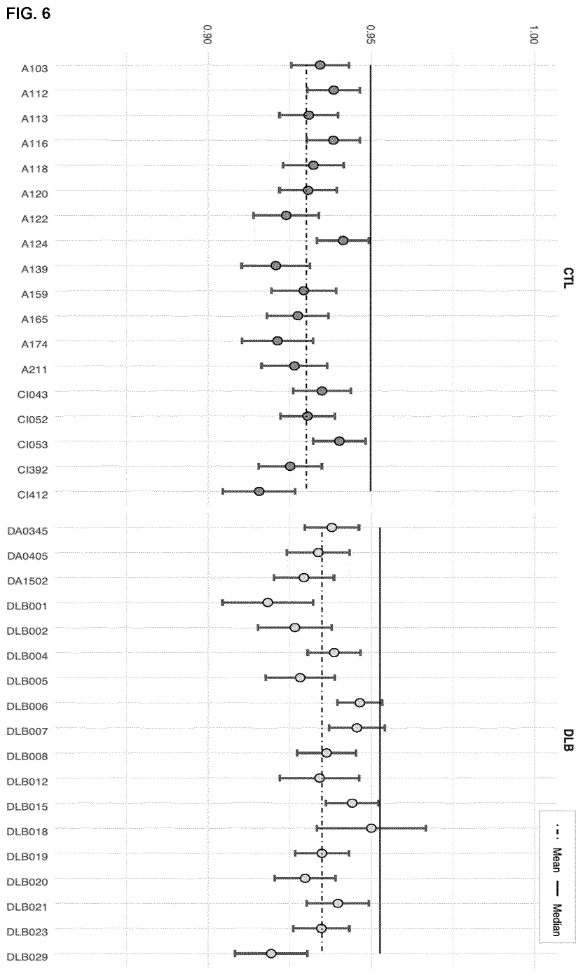

In regard to ND1 gene, results are represented herein in boxplot graphics for all three contexts CpG, CHG and CHH, corresponding to FIG. 1, FIG. 3 and FIG. 5A-5D, respectively. No significant differences in the methylation profile distribution were found when comparing control samples to samples from DLB subjects in any of the three contexts for ND1 gene (adjusted p-value <0.05), as shown in Table 7. Accordingly, mean and confidence interval (95%) graphics, corresponding to the representation of the mean percentages of methylation for each patient, show that there are no significant differences between DLB and CTL subjects in any of the three contexts CpG, CHG and CHH (adjusted p-value <0.05) as represented in FIG. 2, FIG. 4 and FIG. 6, respectively. However, it is worth noting that significant differences were found in all CHH positions of the ND1 gene when statistical significance threshold was set at adjusted p-value <0.1. Thus, the methylation pattern at CHH sites of the ND1 region are indeed different between subjects with DLB and controls.

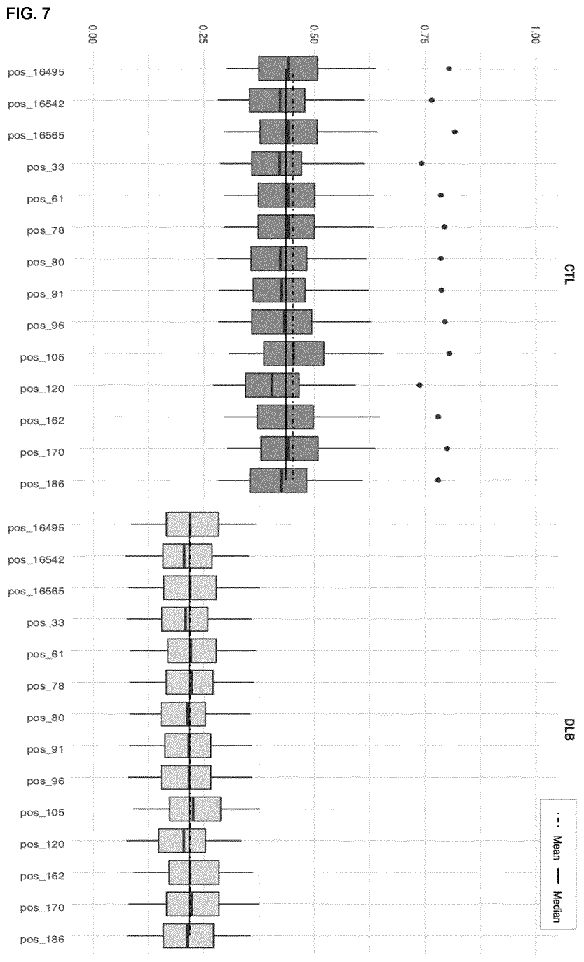

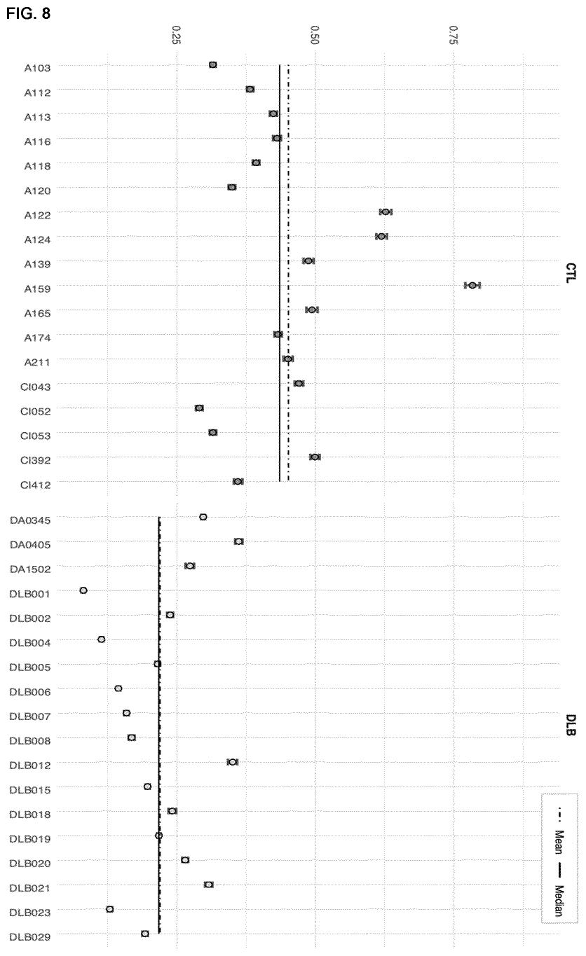

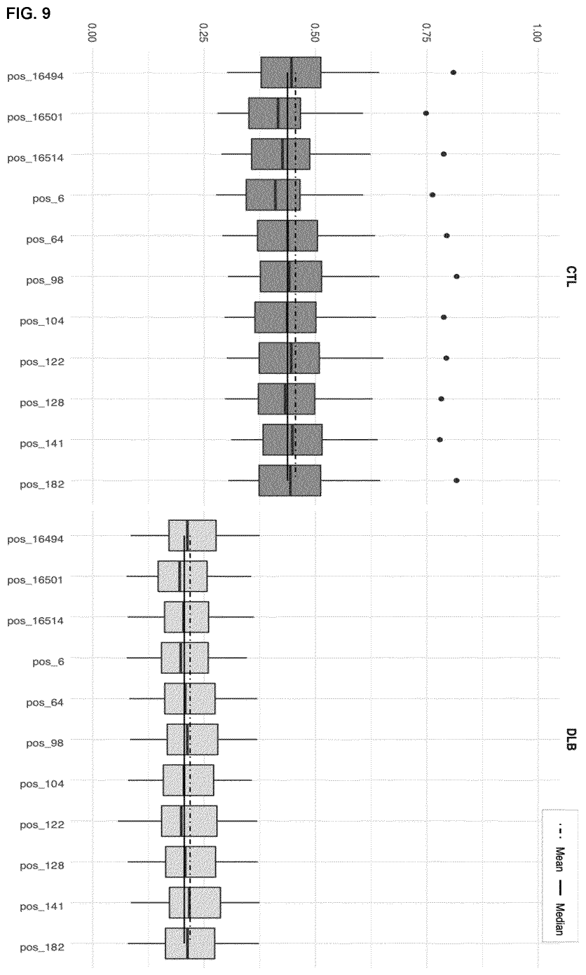

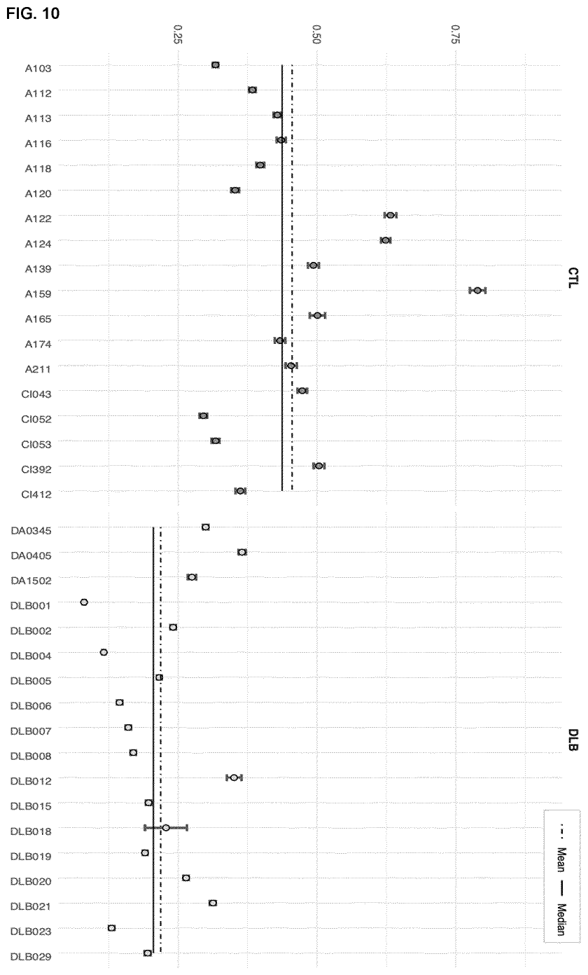

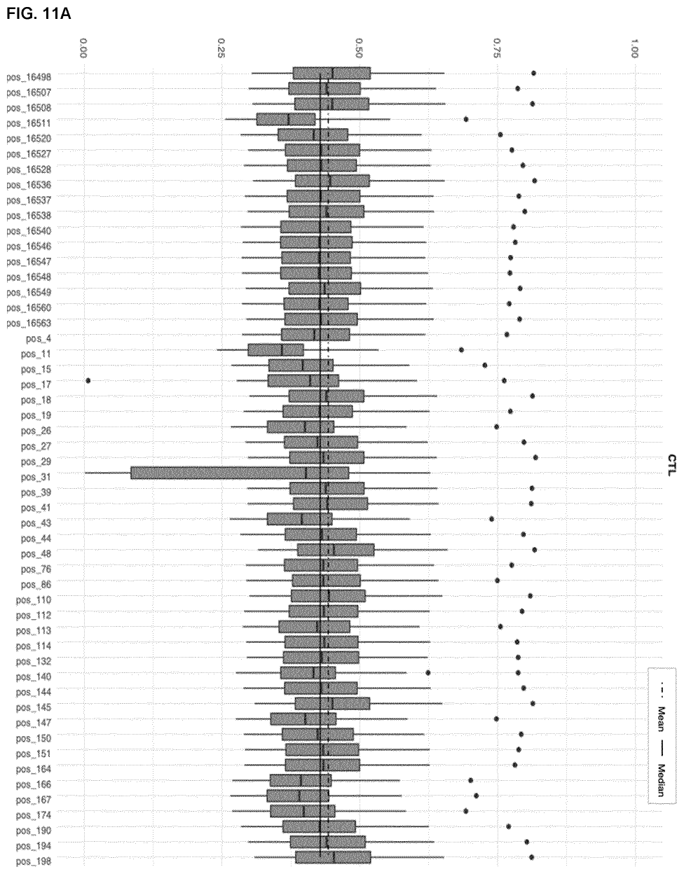

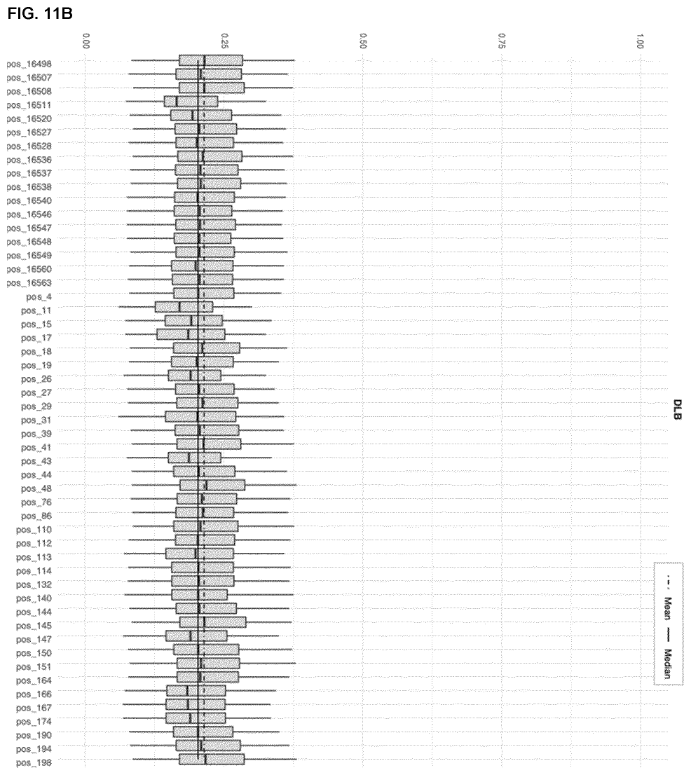

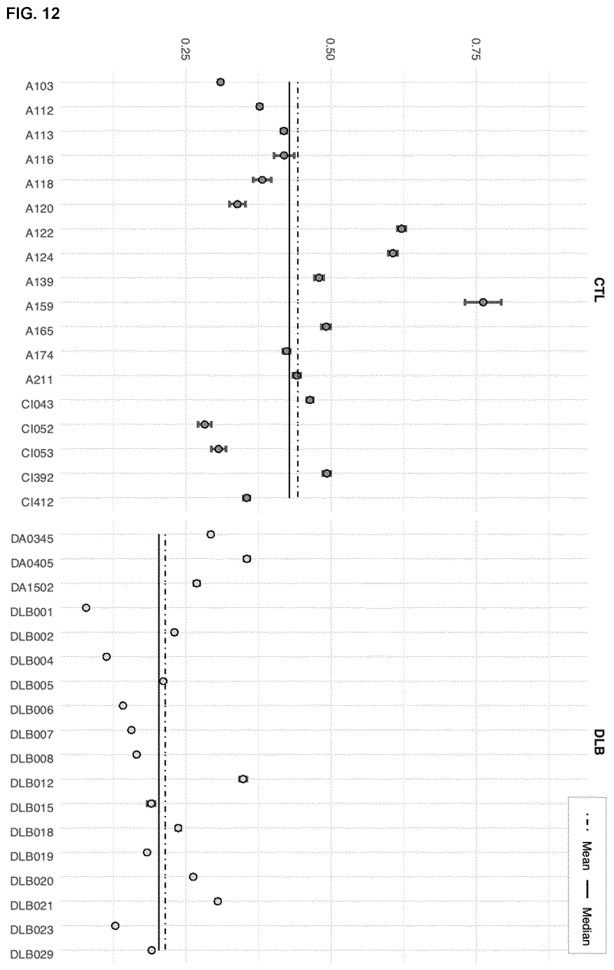

On the other hand, when methylation levels were compared for the three contexts in the D-loop region, highly significant differences were observed. Surprisingly, DLB subjects show very significant hypomethylation compared to control subjects in all three contexts (adjusted p-value <0.05), i.e., CpG, CHG and CHH, as shown in FIG. 7, FIG. 9 and FIG. 11A-B and Table 8, respectively. Additionally, mean and confidence interval (95%) graphics, also indicate a strong and statistically significant difference of the mean percentages of methylation between DLB and control subjects in all three contexts, i.e., CpG, CHG, CHH (FIG. 8, FIG. 10 and FIG. 12, respectively). Notably, as shown in Table 8, adjusted p-values of such differences are considerably under the statistical significance threshold set at adjusted p-value <0.05, with a highest adjusted p-value being only 1.04E-11. Altogether, these results clearly support the existence of a specific mitoepigenetic signatures in the mitochondrial regulatory region D-Loop that characterize DLB subjects and therefore could be considered as promising biomarkers for DLB.

TABLE 7
Adjusted p-values for CpG, CHG and
CHH methylation sites in ND1 gene.

| | | Adjusted |
|---|---|---|
| | | p- |
| | | value |
| C | Position | (FDR) |
| CpG | 95 | 0.150 |
| | 119 | 0.150 |
| | 123 | 0.150 |
| | 150 | 0.150 |
| | 164 | 0.150 |
| | 179 | 0.150 |
| | 197 | 0.150 |
| | 203 | 0.150 |
| | 239 | 0.150 |
| | 269 | 0.150 |
| | 274 | 0.150 |
| | 293 | 0.150 |
| | 386 | 0.150 |
| CHG | 118 | 0.153 |
| | 199 | 0.153 |
| | 238 | 0.153 |
| | 268 | 0.153 |
| | 273 | 0.153 |
| | 333 | 0.153 |
| | 385 | 0.153 |
| | 401 | 0.153 |
| CHH | 31 | 0.090 |
| | 36 | 0.090 |
| | 37 | 0.090 |
| | 39 | 0.090 |
| | 42 | 0.090 |
| | 47 | 0.090 |
| | 50 | 0.090 |
| | 54 | 0.090 |
| | 55 | 0.090 |
| | 56 | 0.090 |
| | 61 | 0.090 |
| | 62 | 0.090 |
| | 65 | 0.090 |
| | 66 | 0.090 |
| | 68 | 0.090 |
| | 69 | 0.090 |
| | 72 | 0.090 |
| | 74 | 0.090 |
| | 75 | 0 090 |
| | 77 | 0.090 |
| | 84 | 0.090 |
| | 85 | 0.090 |
| | 86 | 0.090 |
| | 90 | 0.090 |
| | 97 | 0.090 |
| | 103 | 0.090 |
| | 107 | 0.090 |
| | 108 | 0.090 |
| CHH | 114 | 0.090 |
| | 132 | 0.090 |
| | 137 | 0.090 |
| | 144 | 0.090 |
| | 147 | 0.090 |
| | 152 | 0.090 |
| | 158 | 0.090 |
| | 159 | 0.090 |
| | 160 | 0.090 |
| | 161 | 0.090 |
| | 173 | 0.090 |
| | 174 | 0.090 |
| | 175 | 0.090 |
| | 176 | 0.090 |
| | 183 | 0.090 |
| | 186 | 0.090 |
| | 189 | 0.090 |
| | 192 | 0.090 |
| | 193 | 0.090 |
| | 194 | 0.090 |
| | 205 | 0.090 |
| | 206 | 0.090 |
| | 213 | 0.090 |
| | 215 | 0.090 |
| | 218 | 0.090 |
| | 220 | 0.090 |
| | 221 | 0.090 |
| | 228 | 0.090 |
| | 229 | 0.090 |
| | 230 | 0.090 |
| | 231 | 0.090 |
| | 237 | 0.090 |
| | 241 | 0.090 |
| | 242 | 0.090 |
| | 244 | 0.090 |
| | 247 | 0.090 |
| | 250 | 0.090 |
| | 251 | 0.090 |
| | 254 | 0.090 |
| | 256 | 0.090 |
| | 257 | 0.090 |
| | 258 | 0.090 |
| | 260 | 0.090 |
| | 263 | 0.090 |
| | 266 | 0.090 |
| | 271 | 0.090 |
| | 272 | 0.090 |
| | 277 | 0.090 |
| | 278 | 0.090 |
| CHH | 283 | 0.090 |
| | 285 | 0.090 |
| | 287 | 0.090 |
| | 289 | 0.090 |
| | 290 | 0.090 |
| | 295 | 0.090 |
| | 297 | 0.090 |
| | 300 | 0.090 |
| | 303 | 0.090 |
| | 310 | 0.090 |
| | 311 | 0.090 |
| | 312 | 0.090 |
| | 313 | 0.090 |
| | 314 | 0.090 |
| | 315 | 0.090 |
| | 317 | 0.090 |
| | 318 | 0.090 |
| | 319 | 0.090 |
| | 320 | 0.090 |
| | 324 | 0.090 |
| | 325 | 0.090 |
| | 326 | 0.090 |
| | 329 | 0.090 |
| | 330 | 0.090 |
| | 331 | 0.090 |
| | 332 | 0.090 |
| | 338 | 0.095 |
| | 341 | 0.095 |
| | 342 | 0.095 |
| | 344 | 0.095 |
| | 347 | 0.095 |
| | 348 | 0.095 |
| | 353 | 0.095 |
| | 354 | 0.097 |
| | 356 | 0.097 |
| | 357 | 0.097 |
| | 366 | 0.095 |
| | 370 | 0.095 |
| | 371 | 0.095 |
| | 373 | 0.095 |
| | 374 | 0.095 |
| | 376 | 0.095 |
| | 380 | 0.095 |
| | 381 | 0.095 |
| | 392 | 0.095 |
| | 394 | 0.095 |
| | 398 | 0.095 |
| | 399 | 0.095 |

TABLE 8
Adjusted p-values for CpG, CHG and CHH
methylation sites in the D-loop region.

| | | Adjusted |
|---|---|---|
| | | p- |
| | | value |
| C | Position | (FDR) |
| CpG | 31 | 1.04E−11 |
| | 78 | 1.04E−11 |
| | 101 | 1.04E−11 |
| | 138 | 1.04E−11 |
| | 166 | 1.04E−11 |
| | 183 | 1.04E−11 |
| | 185 | 1.04E−11 |
| | 196 | 1.04E−11 |
| | 201 | 1.04E−11 |
| | 210 | 1.04E−11 |
| | 225 | 1.04E−11 |
| | 267 | 1.04E−11 |
| | 275 | 1.04E−11 |
| | 291 | 1.04E−11 |
| CHG | 30 | 7.26E−12 |
| | 37 | 5.84E−12 |
| | 50 | 5.84E−12 |
| | 111 | 5.84E−12 |
| | 169 | 5.84E−12 |
| | 203 | 5.84E−12 |
| | 209 | 5.84E−12 |
| | 227 | 5.84E−12 |
| | 233 | 5.84E−12 |
| | 246 | 5.84E−12 |
| | 287 | 5.84E−12 |
| CHH | 34 | 1.03E−11 |
| | 43 | 1.03E−11 |
| | 44 | 1.03E−11 |
| | 47 | 1.036−11 |
| | 56 | 1.03E−11 |
| | 63 | 1.03E−11 |
| | 64 | 1.03E−11 |
| | 72 | 1.03E−11 |
| | 73 | 1.03E−11 |
| | 74 | 1.03E−11 |
| | 76 | 1.03E−11 |
| | 82 | 1.03E−11 |
| | 83 | 1.03E−11 |
| | 84 | 1.03E−11 |
| | 85 | 1.03E−11 |
| | 96 | 1.03E−11 |
| | 99 | 1.03E−11 |
| | 109 | 1.03E−11 |
| | 116 | 1.03E−11 |
| | 120 | 1.03E−11 |
| | 122 | 1.03E−11 |
| | 123 | 1.03E−11 |
| | 124 | 1.03E−11 |
| | 131 | 1.03E−11 |
| | 132 | 1.03E−11 |
| | 134 | 1.03E−11 |
| CHH | 136 | 1.03E−11 |
| | 144 | 1.03E−11 |
| | 146 | 1.03E−11 |
| | 148 | 1.03E−11 |
| | 149 | 1.03E−11 |
| | 153 | 1.03E−11 |
| | 181 | 1.03E−11 |
| | 191 | 1.03E−11 |
| | 215 | 1.03E−11 |
| | 217 | 1.03E−11 |
| | 218 | 1.03E−11 |
| | 219 | 1.03E−11 |
| | 237 | 1.03E−11 |
| | 245 | 1.03E−11 |
| | 249 | 1.03E−11 |
| | 250 | 1.03E−11 |
| | 252 | 1.03E−11 |
| | 255 | 1.03E−11 |
| | 256 | 1.03E−11 |
| | 269 | 1.03E−11 |
| | 271 | 1.03E−11 |
| | 272 | 1.03E−11 |
| | 279 | 1.03E−11 |
| | 295 | 1.03E−11 |
| | 299 | 1.03E−11 |
| | 303 | 1.03E−11 |

### Example 2: Development of a Classification Model

The classification model of the present invention is constructed based on two main types of sources of information: (1) clinical variables and (2) methylation measures generated in-house, as described in EXAMPLE 1.

The building procedure is conducted using supervised learning methods. The process consists in four main steps: (1) Exploratory Data Analysis (EDA), (2) pre-processing data, (3) model training process and (4) performance evaluation of the model, as described as follows:

### 1) Exploratory Data Analysis (EDA)

EDA is intended to describe all variables examined in the analysis. For categorical variables, frequency tables and bar diagrams are generated, and for continuous variables, central, dispersion and symmetry measures are estimated. In addition, an histogram and a violin plot is provided for each continuous variable.

### 2) Pre-processing Data

This step is conducted to ensure and enhance the performance of the model training process. Pre-processing data consists of the following six steps:
1. Creating dummy variables using the one-hot encoding approach to guarantee no linear dependencies between the new attributes and thus avoid the dummy variable trap.
2. Removing zero- and near zero-variance variables to avoid instability issues during the fitting process or the model crashing.
3. Splitting the data in two separate datasets; training and testing datasets. The original corrected data is randomly split in two main subsets; one for performing the model training (80% of the samples) and another for validating the classification model (20% of the samples). The random sampling process is driven within each class to preserve the overall class distribution of the data. The percentages are dependent on the results of the EDA.
4. Centering and scaling the two datasets. Continuous variables from the training data set are used to estimate the centering and scaling factors that is then applied on both datasets to generate the normalized datasets for performing the training and testing processes of the classification model
5. Identifying and removing correlated variables, along with cofounding factors. Further, a pairwise correlation analysis based on the Pearson's correlation coefficient is performed to benefit from reducing the level of correlation between the variables. For those pairs showing high levels of absolute correlation values, the variable with the largest mean absolute correlation is removed from the data set. On the other hand, Generalized Linear Models (GLM) is applied to each variable in order to identify and control potential confounding factors. In addition, an unsupervised multivariate approach is carried out to study the relationship among individuals, which described by the set of quantitative and qualitative variables and structured in groups of clinical and molecular data. Consequently, the variables to be included in the model are identified.
6. Examining and visualizing the training data set to review and check any potential no biases from the original data.

### 3) Model Training

Nine supervised learning methods are used to build the classification model. These methods are Linear Discriminant Analysis (LDA), Classification and Regression Trees (CART), k-Nearest Neighbors (KNN), Naive Bayes (NB), Support Vector Machines (SVM) with a linear kernel, Random Forest (RF), Neural Network (NNET), Generalized Boosted Regression Model (GBM), and Binomial Logistic Regression (GLM). These methods have been selected for being supervised classification methods and for their capacity to process both continuous and categorical data. Further, the consideration of importance metrics of these methods is useful to identify key variables that could be modified and used in further analyses. Alternative algorithm methods can be considered for the development of said model training process. All selected methods run using a k-fold Cross-Validation, and the total number of parameter combinations evaluated, iteration times for the backpropagation, and other types of hyperparameters are accommodated and modified during the tuning process.

### 4) Performance Evaluation of the Classification Model

To measure the performance of the training model the following metrics are estimated:
Accuracy: the overall agreement rate averaged over cross-validation iterations.
Kappa: the Cohen's unweighted Kappa statistic averaged across the resampling results.

For each of these statistics mean, median, minimum, maximum, and the first and third quartiles are estimated.

To measure the performance of the classification predictions of the model trained, a confusion matrix is built to show a cross-tabulation of the observed and predicted classes. This table is accompanied by the Accuracy and the Kappa statistic, as well as the common metrics use to evaluated models: Sensitivity, Specificity, Positive Predicted Values, Negative Predicted Values, Precision, Prevalence, FI-score, Detection Rate, and Detection Prevalence. Further, a ROC curve is generated to measure the model performance of classifying subjects with DLB versus CTL subjects.

### Example 3: MtDNA Methylation Patterns with Additional Samples and Development of a Classification Model 2

### 3.1 Mitochondrial DNA Methylation Patterns

A total of 84 samples were analyzed following the methods as described in EXAMPLE 1.

Methylation levels were compared between DLB subjects (N=42) and control subjects (N=42) for two different regions, i.e., ND1 gene and the D-loop region in three different contexts: CpG, CHG and CHH.

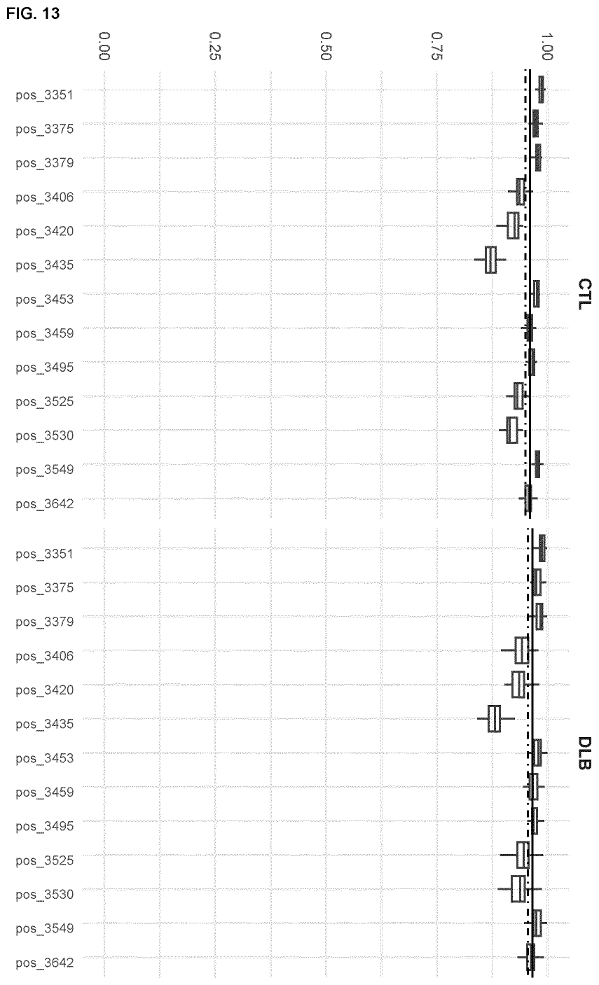

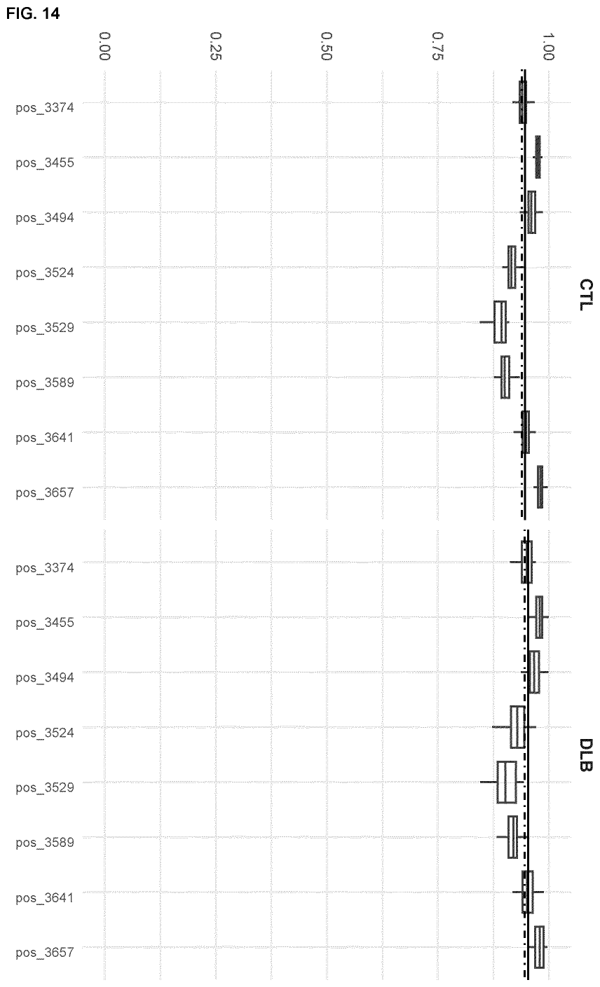

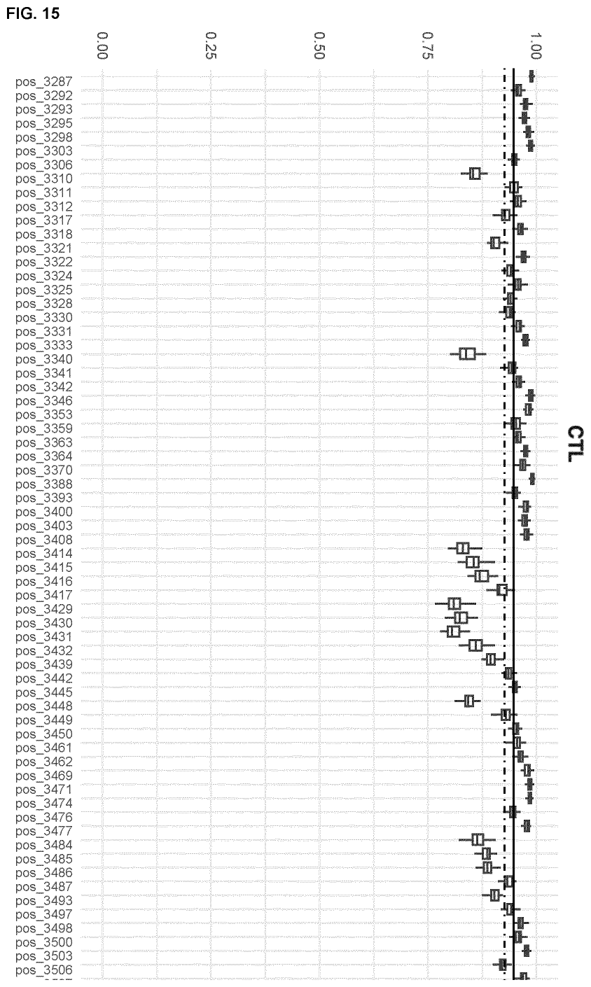

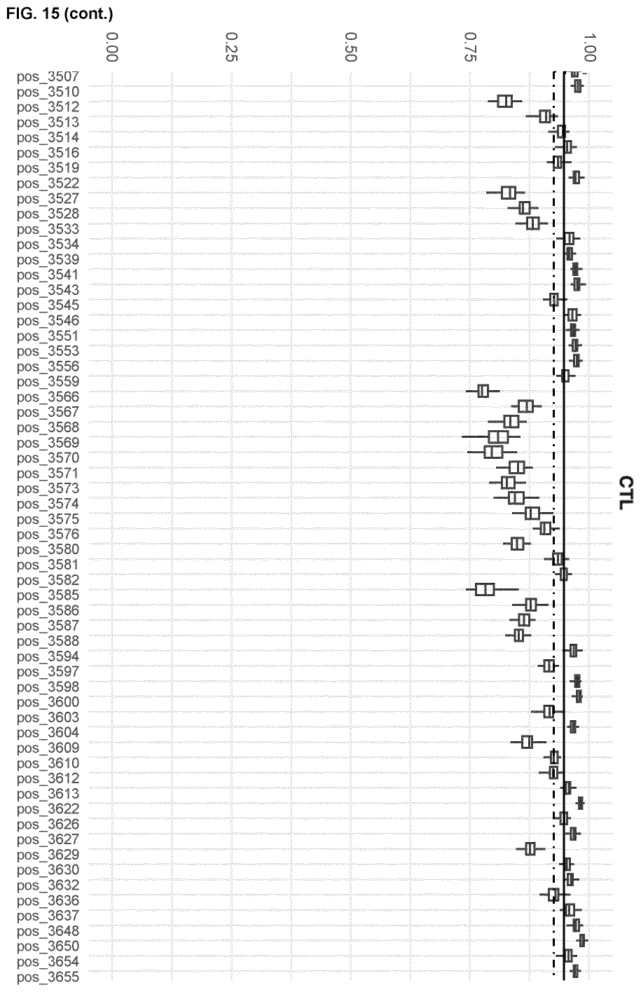

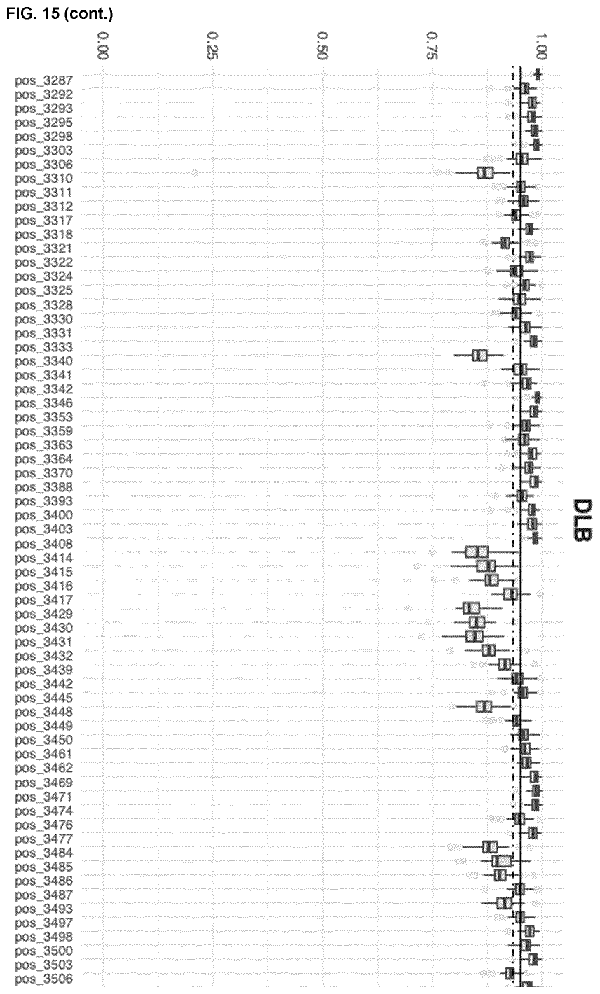

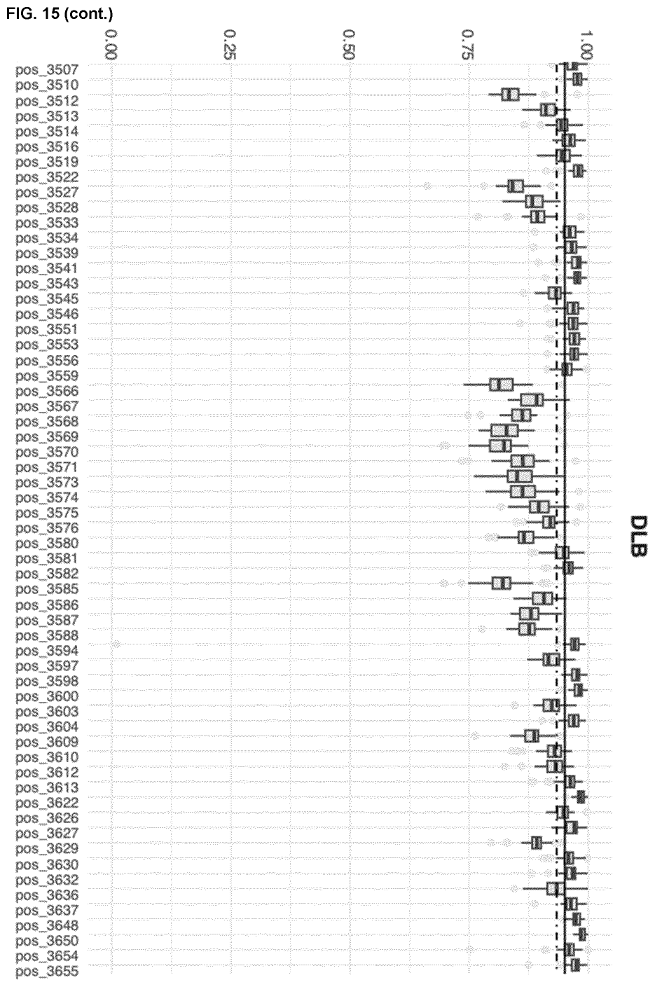

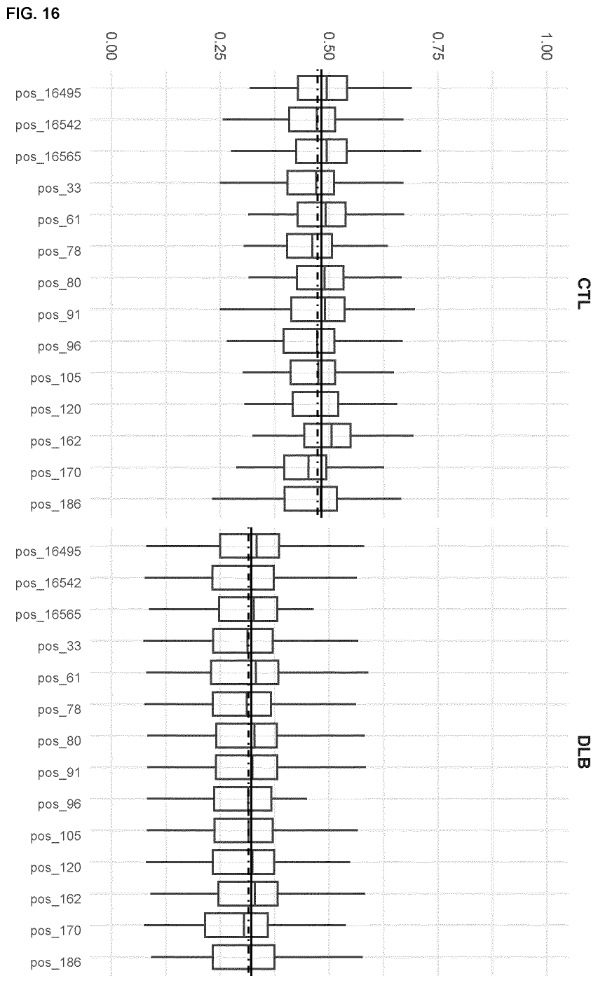

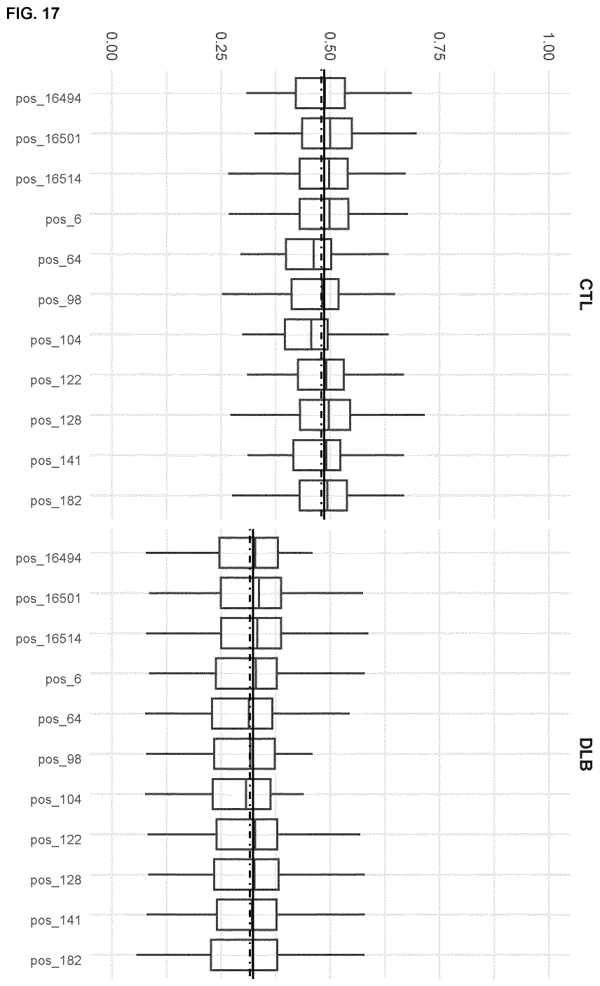

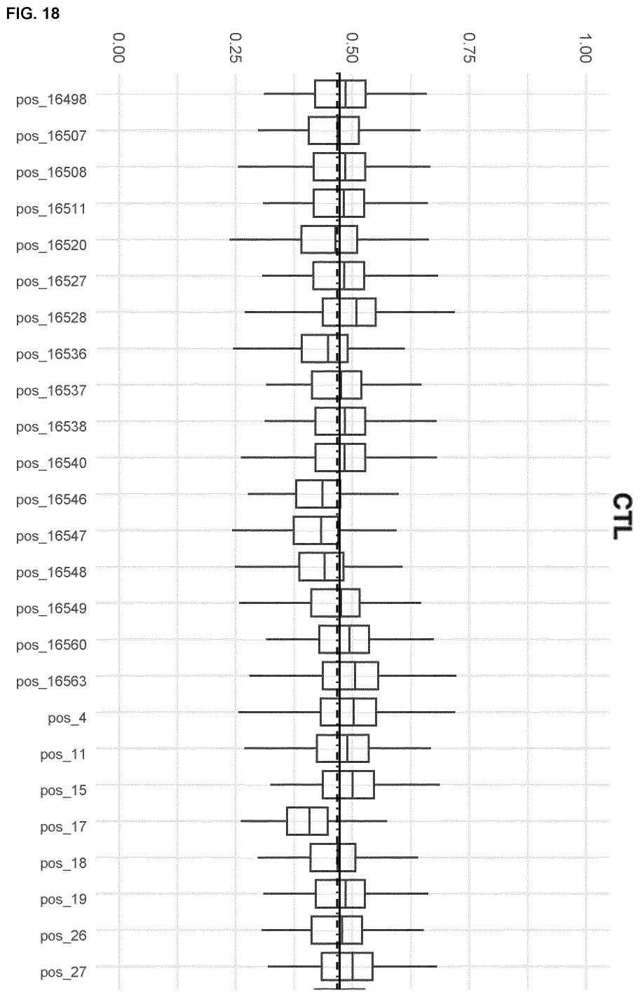

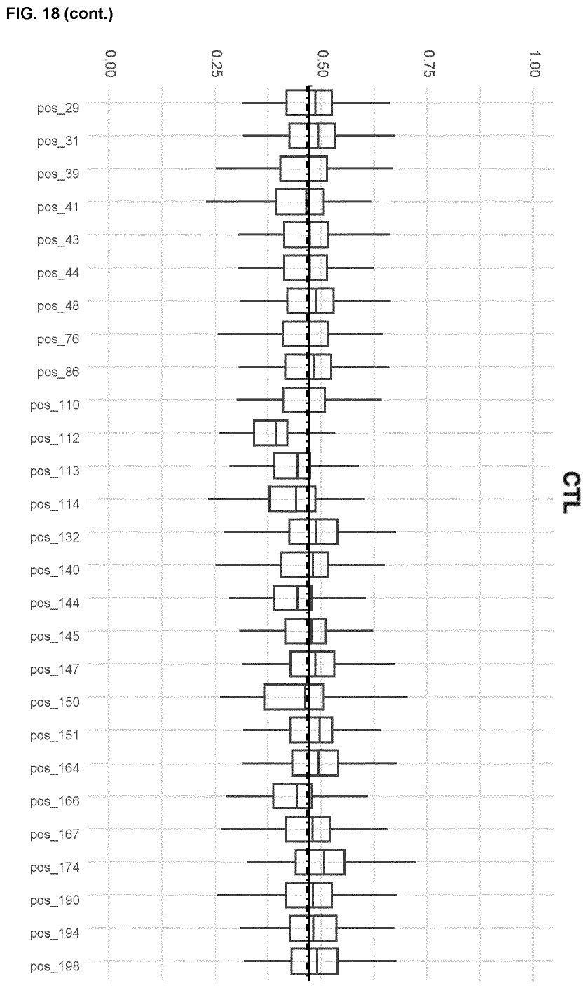

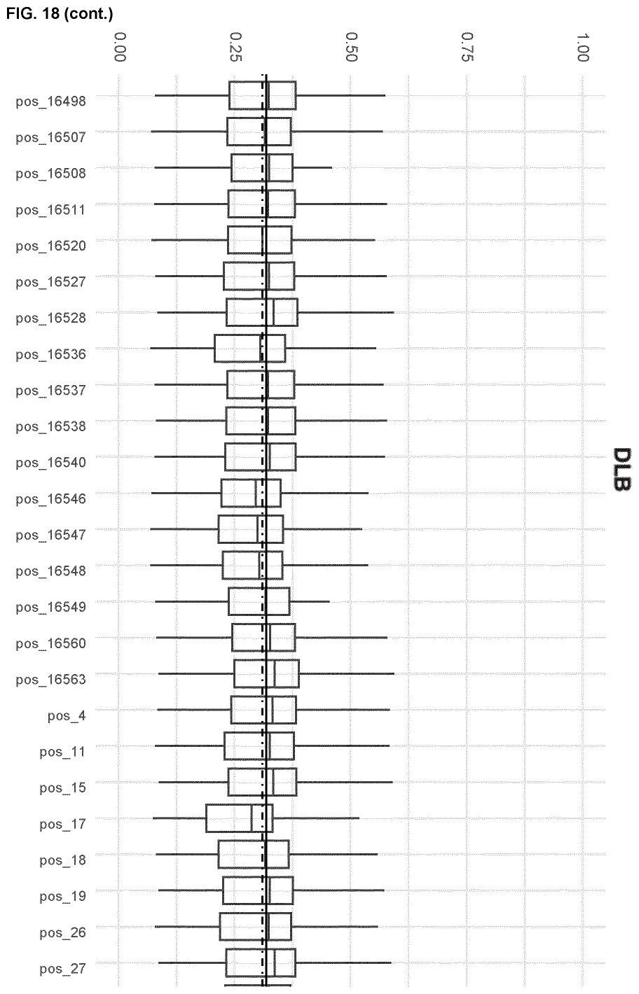

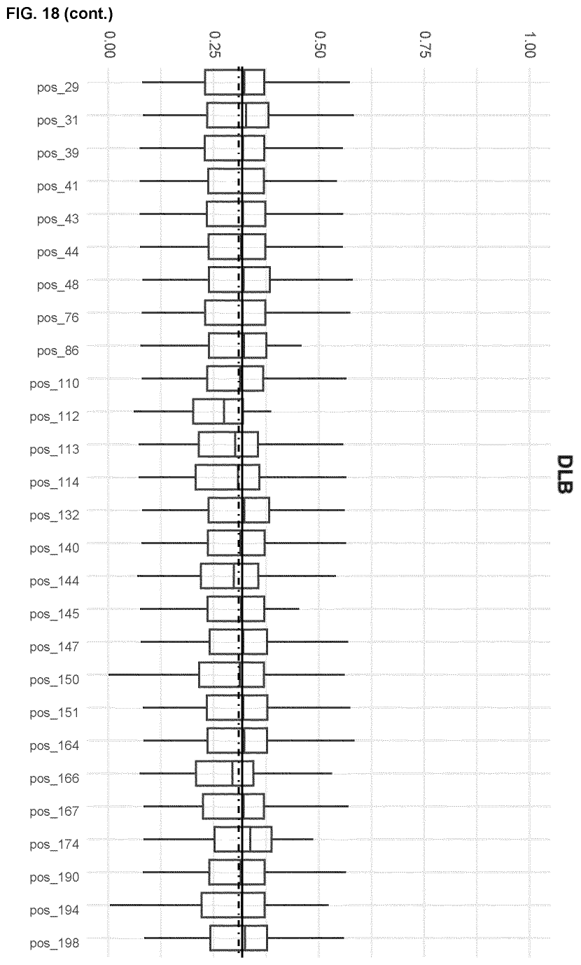

FIGS. 13-18 and Tables 9-10 show the results. In regard to ND1 gene, results are represented herein in boxplot graphics for all three contexts CpG, CHG and CHH. Significant differences in the methylation profile distribution were found when comparing control samples to samples from DLB subjects in all of the three contexts for ND1 gene (adjusted p-value <0.05). Accordingly, mean and confidence interval (95%) graphics, corresponding to the representation of the mean percentages of methylation for each patient, show that there are significant differences between DLB and CTL subjects in all of the three contexts CpG, CHG and CHH (adjusted p-value <0.05).

On the other hand, when methylation levels were compared for the three contexts in the D-loop region, highly significant differences were observed. DLB subjects show very significant differences in methylation compared to control subjects in all three contexts (adjusted p-value <0.05), i.e., CpG, CHG and CHH. Additionally, mean and confidence interval (95%) graphics, also indicate a strong and statistically significant difference of the mean percentages of methylation between DLB and control subjects in all three contexts, i.e., CpG, CHG, CHH. Notably, adjusted p-values of such differences are considerably under the statistical significance threshold set at adjusted p-value <0.05. Altogether these results dearly support the existence of a specific mitoepigenetic signatures in the mitochondrial regulatory region D-Loop and ND1 gene that characterize DLB subjects and therefore could be considered as promising biomarkers for DLB.

TABLE 9
Adjusted p-values for CpG, CHG and
CHH methylation sites in ND1 gene.

| | | Adjusted |
|---|---|---|
| | | p- |
| | | value |
| C | Position | (FDR) |
| CpG | 95 | 8.649E−05 |
| | 119 | 8.649E−05 |
| | 123 | 8.649E−05 |
| | 150 | 8.649E−05 |
| | 164 | 8.649E−05 |
| | 179 | 8.649E−05 |
| | 197 | 8.649E−05 |
| | 203 | 8.649E−05 |
| | 239 | 8.649E−05 |
| | 269 | 8.649E−05 |
| | 274 | 8.649E−05 |
| | 293 | 8.649E−05 |
| | 386 | 0.0001183 |
| CHG | 118 | 7.531E−06 |
| | 199 | 8.412E−05 |
| | 238 | 8.412E−05 |
| | 268 | 8.412E−05 |
| | 273 | 8.412E−05 |
| | 333 | 8.412E−05 |
| | 385 | 6.325E−05 |
| | 401 | 6.325E−05 |
| CHH | 31 | 4.922E−05 |
| | 36 | 5.671E−05 |
| | 37 | 7.184E−05 |
| | 39 | 7.638E−05 |
| | 42 | 9.398E−05 |
| | 47 | 0.0001597 |
| | 50 | 0.0002053 |
| | 54 | 0.0002048 |
| | 55 | 0.0002048 |
| | 56 | 0.0002048 |
| | 61 | 6.27E−05 |
| | 62 | 4.922E−05 |
| | 65 | 4.223E−05 |
| | 66 | 4.223E−05 |
| | 68 | 3.281E−05 |
| | 69 | 3.281E−05 |
| | 72 | 3.281E−05 |
| | 74 | 3.281E−05 |
| | 75 | 3.281E−05 |
| | 77 | 3.281E−05 |
| | 84 | 3.281E−05 |
| | 85 | 3.281E−05 |
| | 86 | 3.281E−05 |
| | 90 | 3.281E−05 |
| | 97 | 3.281E−05 |
| | 103 | 3.281E−05 |
| | 107 | 3.281E−05 |
| | 108 | 3.281E−05 |
| CHH | 114 | 3.281E−05 |
| | 132 | 4.922E−05 |
| | 137 | 4.922E−05 |
| | 144 | 4.922E−05 |
| | 147 | 4.922E−05 |
| | 152 | 4.922E−05 |
| | 158 | 4.922E−05 |
| | 159 | 4.922E−05 |
| | 160 | 4.922E−05 |
| | 161 | 4.922E−05 |
| | 173 | 4.922E−05 |
| | 174 | 4.922E−05 |
| | 175 | 4.922E−05 |
| | 176 | 4.922E−05 |
| | 183 | 4.922E−05 |
| | 186 | 4.922E−05 |
| | 189 | 4.922E−05 |
| | 192 | 4.922E−05 |
| | 193 | 4.922E−05 |
| | 194 | 4.922E−05 |
| | 205 | 4.922E−05 |
| | 206 | 4.922E−05 |
| | 213 | 4.922E−05 |
| | 215 | 4.922E−05 |
| | 218 | 4.922E−05 |
| | 220 | 4.922E−05 |
| | 221 | 4.922E−05 |
| | 228 | 4.922E−05 |
| | 229 | 4.922E−05 |
| | 230 | 4.922E−05 |
| | 231 | 4.922E−05 |
| | 237 | 4.922E−05 |
| | 241 | 4.922E−05 |
| | 242 | 4.922E−05 |
| | 244 | 4.922E−05 |
| | 247 | 4.922E−05 |
| | 250 | 4.922E−05 |
| | 251 | 4.922E−05 |
| | 254 | 4.922E−05 |
| | 256 | 4.922E−05 |
| | 257 | 4.922E−05 |
| | 258 | 4.922E−05 |
| | 260 | 4.922E−05 |
| | 263 | 4.922E−05 |
| | 266 | 4.922E−05 |
| | 271 | 4.922E−05 |
| | 272 | 4.922E−05 |
| | 277 | 4.922E−05 |
| | 278 | 4 922E−05 |
| CHH | 283 | 4.922E−05 |
| | 285 | 4.922E−05 |
| | 287 | 4.922E−05 |
| | 289 | 4.922E−05 |
| | 290 | 4.922E−05 |
| | 295 | 4.922E−05 |
| | 297 | 4.922E−05 |
| | 300 | 4.922E−05 |
| | 303 | 4.784E−05 |
| | 310 | 3.281E−05 |
| | 311 | 3.281E−05 |
| | 312 | 3.281E−05 |
| | 313 | 3.281E−05 |
| | 314 | 3.281E−05 |
| | 315 | 3.281E−05 |
| | 317 | 3.281E−05 |
| | 318 | 3.281E−05 |
| | 319 | 3.281E−05 |
| | 320 | 3.281E−05 |
| | 324 | 3.281E−05 |
| | 325 | 3.281E−05 |
| | 326 | 3.281E−05 |
| | 329 | 3.281E−05 |
| | 330 | 3.281E−05 |
| | 331 | 3.281E−05 |
| | 332 | 3.281E−05 |
| | 338 | 3.281E−05 |
| | 341 | 3.281E−05 |
| | 342 | 3.281E−05 |
| | 344 | 3.281E−05 |
| | 347 | 3.281E−05 |
| | 348 | 3.281E−05 |
| | 353 | 3.281E−05 |
| | 354 | 3.281E−05 |
| | 356 | 3.281E−05 |
| | 357 | 3.281E−05 |
| | 366 | 3.281E−05 |
| | 370 | 3.281E−05 |
| | 371 | 3.281E−05 |
| | 373 | 3.281E−05 |
| | 374 | 3.281E−05 |
| | 376 | 3.281E−05 |
| | 380 | 3.281E−05 |
| | 381 | 3.281E−05 |
| | 392 | 3.281E−05 |
| | 394 | 3.281E−05 |
| | 398 | 3.281E−05 |
| | 399 | 3.281E−05 |

TABLE 10
Adjusted p-values for CpG, CHG and CHH
methylation sites in the D-loop region.

| | | Adjusted |
|---|---|---|
| | | p- |
| | | value |
| C | Position | (FDR) |
| CpG | 31 | 7.544E−11 |
| | 78 | 7.544E−11 |
| | 101 | 7.544E−11 |
| | 138 | 7.544E−11 |
| | 166 | 7.544E−11 |
| | 183 | 7.544E−11 |
| | 185 | 7.544E−11 |
| | 196 | 7.544E−11 |
| | 201 | 7.544E−11 |
| | 210 | 7 544E−11 |
| | 225 | 7.544E−11 |
| | 267 | 7.544E−11 |
| | 275 | 7.544E−11 |
| | 291 | 7.544E−11 |
| CHG | 30 | 5.581E−11 |
| | 37 | 5.116E−11 |
| | 50 | 5 116E−11 |
| | 111 | 5.116E−11 |
| | 169 | 5.116E−11 |
| | 203 | 5.116E−11 |
| | 209 | 5.116E−11 |
| | 227 | 5.116E−11 |
| | 233 | 5.116E−11 |
| | 246 | 5.116E−11 |
| | 287 | 5.116E−11 |
| CHH | 34 | 1.091E−10 |
| | 43 | 1.091E−10 |
| | 44 | 1.091E−10 |
| | 47 | 1.091E−10 |
| | 56 | 1.031E−10 |
| | 63 | 1.031E−10 |
| | 64 | 1.031E−10 |
| | 72 | 1.031E−10 |
| | 73 | 1.031E−10 |
| | 74 | 1.031E−10 |
| | 76 | 1.031E−10 |
| | 82 | 1.031E−10 |
| | 83 | 1.031E−10 |
| | 84 | 1.031E−10 |
| | 85 | 1.031E−10 |
| | 96 | 1.031E−10 |
| | 99 | 1.031E−10 |
| | 109 | 1.031E−10 |
| | 116 | 1.031E−10 |
| | 120 | 1.031E−10 |
| | 122 | 1.031E−10 |
| | 123 | 1.031E−10 |
| | 124 | 1.031E−10 |
| | 131 | 1.031E−10 |
| | 132 | 1.031E−10 |
| | 134 | 1.031E−10 |
| CHH | 136 | 1.031E−10 |
| | 144 | 1.031E−10 |
| | 146 | 1.031E−10 |
| | 148 | 1.031E−10 |
| | 149 | 1.031E−10 |
| | 153 | 1.031E−10 |
| | 181 | 1.031E−10 |
| | 191 | 1.031E−10 |
| | 215 | 1.031E−10 |
| | 217 | 1.031E−10 |
| | 218 | 1.031E−10 |
| | 219 | 1.031E−10 |
| | 237 | 1.031E−10 |
| | 245 | 1.031E−10 |
| | 249 | 1.031E−10 |
| | 250 | 1.031E−10 |
| | 252 | 1.031E−10 |
| | 255 | 1.031E−10 |
| | 256 | 1.031E−10 |
| | 269 | 1.031E−10 |
| | 271 | 1.031E−10 |
| | 272 | 1.031E−10 |
| | 279 | 1.031E−10 |
| | 295 | 1.031E−10 |
| | 299 | 1.031E−10 |
| | 303 | 1.031E−10 |

### 3.2. Development of a Classification Model.

A prototype model to classify subjects diagnosed with DLB was developed.

### Materials and Methods

### 1) Target Data

Raw data was checked to ensure the performance of the modeling process. For this reason, subjects with missing values were excluded from the analysis. In this way, a total of 84 subjects recruited from:
ADmit cohort (15 subjects): Hospital Universitari de Bellvitge, Barcelona-Spain;
AIBL cohort (39 subjects): Australian Imaging, Biomarker & Lifestyle Flagship Study of Ageing, Australia;
CITA cohort (3 subjects): Center for Research and Advanced Therapies, CITA-Alzheimer Foundation,
Donostia-San Sebastian, Spain;
Hospital Clinic cohort (3 subjects): Hospital Clinic de Barcelona, Spain; and
MAP-AD cohort (24 subjects): Hospital Universitari de Bellvitge, Hospital Clinic de Barcelona, Hospital General de l'Hospitalet, and Hospital de Sant Joan Despi Moises Broggi, from Barcelona, Spain.

In this regard, two groups of individuals were considered:
Controls (CTL): 42 (50.0%) subjects with:
   a Clinical Dementia Rating (CDR) score of 0 and
   a clinical follow-up longer than 10 years
DLB: 42 (50.0%) subjects with:
   having diagnosis with DLB.

Control subjects were only available from AIBL and CITA cohorts, and patients from ADmit, Hospital Clinic and MAP-AD cohorts. Two main types of sources of information were considered for conducting the analysis, (1) clinical variables and (2) methylation measures generated in-house. Clinical variables were used to describe the data considered for the analysis, that is, only methylation measure were involved in the construction of the classification model.

### 2) Clinical Variables

In this first prototype, 3 clinical variables were examined:
Stage (response variable): consists of three levels as mentioned above Controls and DLB.
Sex: indicates two gender levels Female and Male.
Age: patient's age at the first medical consultation.

### 3) Methylation Measures

224 variables, each of them gathering the percentage of methylation for a single specific cytosine site in one of the three contexts (i.e, CpG, CHG, and CHH) for each gene (i.e., Dloop, and ND1) as described in EXAMPLE 1.

### 4) Methods

A work flow split into four main steps was implemented, which were:
exploratory data analysis,
pre-processing data,
model training process and
evaluating the performance.

### 5) Exploratory Data Analysis

The Exploratory Data Analysis (EDA) was intended to describe every single variable examined in the analysis. For categorical variables, frequency tables and bar diagrams were generated. For continuous variables, central (mean, median, and mode where applicable), dispersion (standard deviation, standard error, median absolute deviation, and range) and symmetry (skewness and kurtosi) measures were estimated. In addition, for each continuous variable, an histogram and a violin plot were provided.

### 6) Data Pre-Processing

This step was conducted to ensure and enhance the performance of the model training process. It consisted of splitting the data into a training and testing data sets, centering and scaling both data sets, identifying appropriate features variables, examining, and visualizing the training data set.
1. Data splitting: The original corrected data was randomly split into to main subsets; one for performing the model training (80% of the samples) and other for testing the classification model (20% of the samples). The random sampling process was driven within each class to preserve the overall class distribution of the data.
2. Centering and Scaling: Continuous variables from the training data set were used to estimate the centering and scaling factors that were applied on both data sets to generate the normalized data sets for performing the training and testing processes of the classification model.
3. Identifying and Removing Correlated Variables: To benefit from reducing the level of correlation between variables, a pairwise correlation analysis based on the Spearman's rank correlation coefficient was performed. For those pairs showing high levels of absolute correlation values (>0.65), the variable with the largest mean absolute correlation was removed from the data set. Furthermore, a Principal Component Analysis (PCA) was applied to reduce the dimensionality of the features. In this case, the top 10 variables were considered.

Examining and visualizing the training data set: After applying the previous pre-processing task a second EDA was applied on the training data set in order to review and check no biases from the original data.

### 7) Model Training

Ten supervised learning methods were selected to build the prototype. These methods were:
GLMNET: Generalized Linear Model via Penalized Maximum Likelihood (Logistic Regression)
LDA: Linear Discriminant Analysis
PMR: Penalized Multinomial Regression
AdaBoost: AdaBoost Classification Trees
CART: Classification and Regression Trees
kNN: k-Nearest Neighbors
NB: Naive Bayes.
SVM: Support Vector Machines with a linear kernel.
RF: Random Forest
NNET: Neural Network

They were selected for three main reasons:
1. These approaches are supervised classification methods
2. They are capable of handling continuous and categorical data
3. The importance metrics might help to identify key variables that could be modified and used in further analyses.

All methods were run using a 3 repeated 10-fold Cross-Validation. The total number of parameter combinations evaluated in SVM, RF and NNET was 5. NNET was estimated using a back propagation approach iterating 1000 times.

### 8) Evaluation of the Performance of the Classification Model

To measure the performance of the training model the following metrics were estimated:
Accuracy: the overall agreement rate averaged over cross-validation iterations.
Kappa: the Cohen's unweighted Kappa statistic averaged across the resampling results.

For each of these statistics mean, median, minimum, maximum, as well as the first and third quartiles, were estimated. To measure the performance of the classification predictions a confusion matrix was

#####built to show a cross-tabulation of the observed and predicted classes. This table was accompanied by Accuracy and the Kappa statistic, as well as the common metrics used to evaluate models Sensitivity, Specificity, Positive Predicted Values, Negative Predicted Values, Precision, _Prevalence, FI score, Detection Rate,—Detection Prevalence. Also, for measuring model performance of classifying DLB versus control patients a ROC curve was built.

TABLE 11
Summary descriptive tables of clinical variables

| | Sex | |
|---|---|---|
| Stage | F | N |
| CTL | 17 (40.48%) | 25 (59.52%) |
| DLB | 18 (42.86%) | 24 (57.14%) |
| Total | 24 (57.14%) | 49 (58.33%) |

| Stage | Count | Min | Max | Mean | Media [symbol] | 1st | 3rd | IQR | St | Varianc [symbol] |
|---|---|---|---|---|---|---|---|---|---|---|
| CTL | 42 | 62 | 87 | 70.57 | 70 | 67.00 | 73.00 | 6.0 | 5.4 | 29.13 |
| DLB | 42 | 59 | 85 | 73.45 | 74 | 69.25 | 76.75 | 7.5 | 5.4 | 29.13 |

[symbol]

indicates data missing or illegible when filed

Count number of individuals; 1st Qu: first quartile; 3rd Qu: third quartile; IQR interquartile ranges St Dev: standard deviation

### Results

### Exploration Data Analysis

Briefly, according to the EDA results, Sex showed a balanced number of subjects 35 (41.67%) females and 49 (58.33%) males. The percentage of gender remained balanced in each Stage group, 17 (40.48%) females and 25 (59.52%) males for Controls and 18 (42.86%) females and 24 (57.14%) males for DLB. In addition, recruited individuals were 72.01±5.56 years old. Age for subjects of each Stage were 70.57±5.4 years old for Controls and 73.45±5.4 years old for DLB.

### Pre Processing Data

Data splitting generated a first subset of 69 subjects for the training model process and a second subset of 16 individuals for testing the classification model. More than 2900 pairs of variables were identified to have an absolute correlation higher than 0.65. This fact led to remove more than 40% of the quantitative variables from the data, and left with 132 potential predictors. After applying the correlation approach a PCA was applied in order to reduce the dimensionality of the variables.

### Model Training

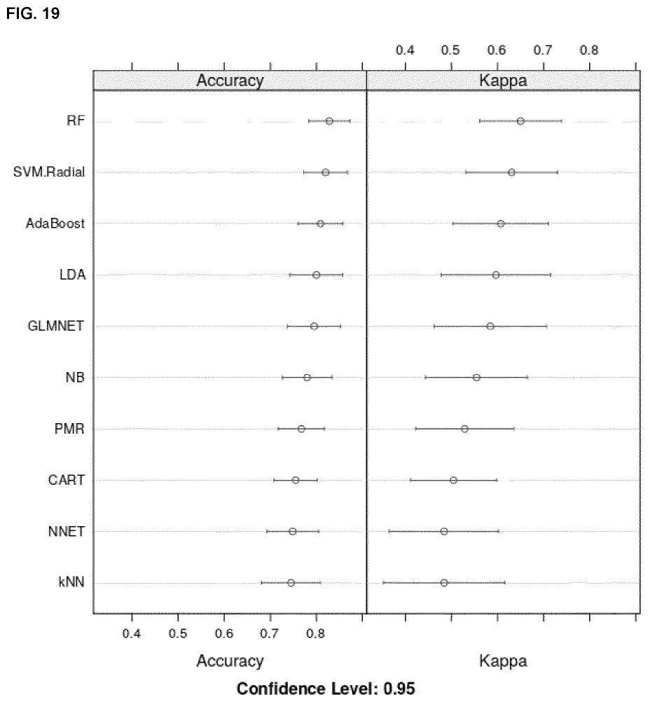

Results of the training process indicates that the best performance seems to be the RF model, where the average accuracy is 0.83 and the Kappa value is 0.65. These values suggest that it is a good model for class tying the DLB patients, Tables 12 and 13 show the accuracy and Kappa metrics of the training models that have been implemented, and FIG. 19 shows a summary of these tables

TABLE 12
Accuray metrics of supervised learning models that have been trained.

| Training | | | | | | | |
|---|---|---|---|---|---|---|---|
| [symbol] | Min. | 1st. Qu. | Median | Mean | 3rd. Qu. | Max | NA' [symbol] |
| GLMNET | 0.4285714 | 0.71428 | 0.83333 | 0. 79503 | 0.87500 | 1.000 | 0 |
| LDA | 0.4285714 | 0.71428 | 0.83333 | 0.80000 | 0.87500 | 1.000 | 0 |
| PMR | 0.4285714 | 0 71428 | 0.75000 | 0.76746 | 0.85119 | 1.000 | 0 |
| AdaBoost | 0.5714286 | 0.71428 | 0.83333 | 0.80912 | 0.87053 | 1.000 | 0 |
| CART | 0 4285714 | 0.67857 | 0.83333 | 0.75496 | 0 85714 | 0.875 | 0 |
| kNN | 0.4285714 | 0.58482 | 0.71428 | 0.74484 | 0.85714 | 1.000 | 0 |
| NB | 0.5000000 | 0.66666 | 0.79166 | 0.78015 | 0.85714 | 1.000 | 0 |
| SVM Radial | 0.5714286 | 0.71428 | 0 83333 | 0.82003 | 0.87053 | 1.000 | 0 |
| RF | 0.5714286 | 0.71428 | 0.83333 | 0 82817 | 0.87500 | 1.000 | 0 |
| NNET | 0.4285714 | 0.63541 | 0.83333 | 0.74861 | 0.85714 | 1.000 | 0 |
| | | [symbol] | [symbol] | [symbol] | [symbol] | | |

NA's: Not available. [symbol]
indicates data missing or illegible when filed

TABLE 13
Kappa metrics of supervised learning models that have been trained.

| Training | Min. | 1st. Qu. | Median | Mean | 3rd. Qu. | Max | NA' [symbol] |
|---|---|---|---|---|---|---|---|
| GLMNET | — | 0.41666 | 0.66666 | 0.58446 | 0.75000 | 1.000 | 0 |
| LDA | — | 0.41666 | 0.66666 | 0.59671 | 0.75000 | 1.000 | 0 |
| PMR | — | 0.41666 | 0.50000 | 0.52871 | 0.68840 | 1.000 | 0 |
| AdaBoost | 0.0000000 | 0.42788 | 0.66666 | 0.60710 | 0.74250 | 1.000 | 0 |
| CART | — | 0.34090 | 0.66666 | 0.50442 | 0.69565 | 0.75 | 0 |
| kNN | — | 0.22916 | 0.39015 | 0.48411 | 0.72000 | 1.000 | 0 |
| NB | 0.0000000 | 0.33333 | 0.58333 | 0.55418 | 0.72000 | 1.000 | 0 |
| SVM Radial | 0.0869565 | 0.41666 | 0.66666 | 0.63074 | 0.74250 | 1.000 | 0 |
| RF | 0.2222222 | 0.42788 | 0.66666 | 0.65009 | 0.75000 | 1.000 | 0 |
| NNET | — | 0.27083 | 0.66666 | 0.48384 | 0.69565 | 1.000 | 0 |

NA's: Not available. [symbol]
indicates data missing or illegible when filed

### Evaluating the Performance of the Model

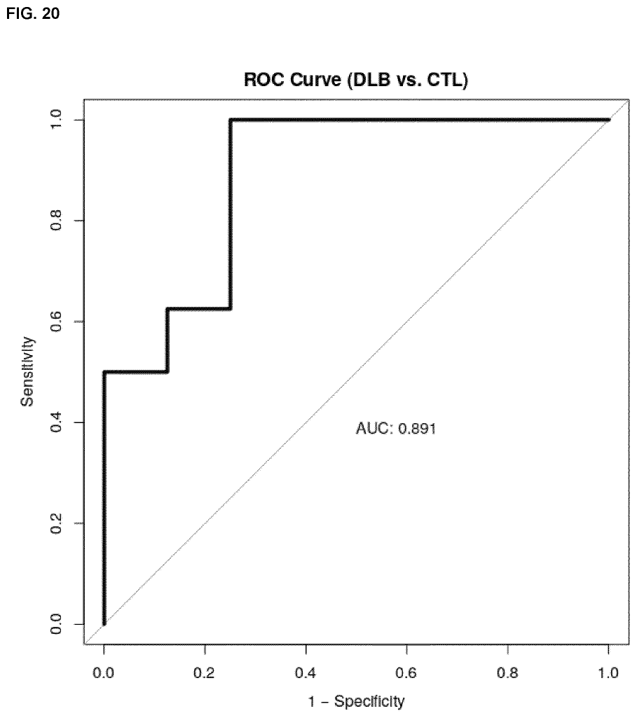

Accuracy of the classification on the testing data of the RF model came out to 0.81, a 95% Confidence Interval of 0.54 to 0.96, and a Kappa value of 0.63. Sensitivity and Specificity of the model to classify a subject as a DLB were 0.75 and 0.875, respectively. The precision of the model 0.86, and the FI-score was 0.8. FIG. 20 displays the ROC curve showing the performance of the classification model for the DLB patients at all classification thresholds.

In summary, the present prototype of classification model seems to perform a good job of identifying DLB patients. Nonetheless, it requires a review with a higher sample size to improve the tuning process during the model training. This fact might cause that estimates provided in this report as well as its associated metrics to evaluate the performance of the classification could be slightly modified.

### REFERENCES

### Non-Patent Literature

De Boni, L., Tierling, S., Roeber, S., Walter, J., Giese, A., & Kretzschmar, H. A. (2011). Next-generation sequencing reveals regional differences of the a-synuclein methylation state independent of Lewy body disease. NeuroMolecular Medicine, 13(4), 310-320.
Desplats, P., Spencer, B., Coffee, E., Patel, P., Michael, S., Patrick, C., Adame, A., Rockenstein, E., & Masliah, E. (2011), a-synuclein sequesters Dnmt1 from the nucleus. A novel mechanism for epigenetic alterations in Lewy body diseases. Journal of Biological Chemistry, 286(11); 9031-9037.
Funahashi, Y., Yoshino, Y., Yamazaki, K., Mori, Y., Mori, T., Ozaki, Y., Sao, T., Ochi, S., Iga, J. I., & Ueno, S. I. (2017). DNA methylation changes at SNCA intron 1 in patients with dementia with Lewy bodies. Psychiatry and Clinical Neurosciences, 71(1), 28-35.
Urbizu, A., & Beyer, K. (2020). Epigenetics in lewy body diseases: Impact on gene expression, utility as a biomarker, and possibilities for therapy. International Journal of Molecular Sciences, 21(13), 1-31.
Chouliaras, L., Kumar, G. S., Thomas, A. J., Lunnon, K., Chinnery, P. F., & O'Brien, J. T. (2020). Epigenetic regulation in the pathophysiology of Lewy body dementia. Progress in Neurobiology, 192.
Fernandez, A. F., Assenov, Y., Martin-Subero, J. I., Balint, B., Siebert, R., Taniguchi, H., Yamamoto, H., Hidalgo, M., Tan, A. C., Galm, O., Ferrer, I., Sanchez-Cespedes, M., Villanueva, A., Carmona, J., Sanchez-Mut, J. V., Berdasco, M., Moreno, V., Capella, G., Monk, D., Esteller, M. (2012). A DNA methylation fingerprint of 1628 human samples. Genome Research, 22(2), 407-419.
Sanchez-Mut, J. V., Heyn, H., Vidal, E., Moran, S., Sayols, S., Delgado-Morales, R., Schultz, M. D., Ansoleaga, B., Garcia-Esparcia, P., Pons-Espinal, M., De Lagran, M. M., Dopazo, J., Rabano, A., Avila, J., Dierssen, M., Lott, I., Ferrer, I., Ecker, J. R., & Esteller, M. (2016). Human DNA methylomes of neurodegenerative diseases show common epigenomic patterns. Translational Psychiatry, 6(1), e718-8.
Nasamran, C. A., Sachan, A. N. S., Mott, J., Kuras, Y. I., Scherzer, C. R., Ricciardelli, E., Jepsen, K., Edland, S. D., Fisch, K. M., & Desplats, P. (2020). Differential blood DNA methylation across lewy body dementias. Alzheimer's and Dementia: Diagnosis, Assessment and Disease Monitoring, 13(1), 1-12.
Blanch, M., Mosquera, J L., Ansoleaga, B., Ferrer, I., Barrachina, M. (2016). Altered Mitochondrial DNA methylation pattern in Alzheimer Disease-related pathology and in Parkinson disease. The American Journal of Pathology, 186(2):385-97.
Stoccoro, A., Siciliano, G., Migliore, L, Coppede, F. (2017). Decreased methylation of the mitocondrial D-Loop region in late-onset Alzheimer's disease, Journal of Alzheimer's Disease, 59(2):559-564.
Stoccoro, A., Baldacci, F., Copped& F., Migliore; L. (2022) Mitochondrial DNA methylation levels are altered in individuals with mild cognitive impairment, Abstract OO016/#1584 On-demand symposium: AD diagnosis & clinical trials & advances in drug development 1.

### Patent Literature

WO2015/144964 A2

1. A method for identifying dementia with Lewy bodies in a subject, comprising:
(a) determining in a sample of the subject comprising mitochondrial DNA, the methylation pattern in the D-loop region and/or the ND1 gene of the mitochondrial DNA,
wherein the methylation pattern is determined in at least one site selected from the group consisting of:
(i) the CpG sites of the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2, and
(v) the CHG sites of the ND1 gene shown in Table 4,
and wherein the methylation pattern is determined in at least one of (vi) the CHH sites of the ND1 gene shown in Table 6.

2. The method according to claim 1, wherein the methylation pattern is determined in all CHH sites of the ND1 gene shown in Table 6.

3. The method according to claim 1, wherein the methylation pattern is determined in all CHG sites of the D-loop region gene shown in Table 3.

4. The method according to claim 1, wherein hypomethylation in at least one site of said CpG sites in the D-loop region, wherein hypomethylation in at least one site of said CHG sites in the D-loop region and/or wherein hypomethylation in at least one site of said CHH sites in the D-loop region is indicative that the subject suffers from dementia with Lewy bodies.

5. The method according to claim 1, wherein the methylation pattern is determined in all CpG, CHG and CHH sites in the D-loop region shown in Tables 1, 3 and 5.

6. The method according to claim 1, wherein the methylation pattern is determined using at least one oligonucleotide capable of specifically hybridizing with a mitochondrial DNA sequence comprising at least one methylation site selected from the group consisting of (i)-(vi), wherein the oligonucleotides have a length between 15 and 100 nucleotides, and are capable of specifically hybridizing with a mitochondrial DNA sequence comprising nucleotides from 16,465 to 230 of NCBI Reference Sequence: NC_012920.1 corresponding to D-loop region and/or with a mitochondrial DNA sequence comprising nucleotides from 3,257 to 3,682 of NCBI Reference Sequence: NC_012920.1 corresposing to ND1 gene.

7. The method according to claim 6, wherein the oligonucleotides are degenerated oligonucleotides.

8. The method according to claim 7, wherein the at least one oligonucleotide is selected from the group consisting of SEQ ID NO: 1, SEQ ID NO: 2, SEQ ID NO: 3 and SEQ ID NO: 4.

9. The method according to claim 1, wherein determining the methylation pattern is determined by bisulfite sequencing.

10. The method according to claim 1, wherein the method further comprises:
(b) combining the methylation pattern of one or more sites determined in step (a), optionally with at least one clinical variable of the subject selected from the group consisting of: demographic variables, neuropsychological variables, clinical observations variables and clinical tests variables,
wherein said combining is performed using a classification model for determining a score which correlates to the identification of dementia with Lewy bodies in the subject.

11. The method according to claim 10, wherein the at least one clinical variable of the subject is selected from the group consisting of: sex, age, race, scholarship, family history, praxis tests, Luria's tests, Clinical Dementia Rating, Global Deterioration Scale, Mini-Mental State Exam, Clinical Dementia Rating Scale Sum of Boxes, neuroleptic intolerance, REM sleep behavior disorder, dysautonomia, parkinsonism, visual hallucinations, cognitive fluctuations, magnetic resonance imaging, Dopamine Transporter Scan, positron emission tomography with 18F-fluorodeoxyglucose, amyloid positron emission tomography, apolipoprotein E genotype, alE4, β-42, tau-T and tau-P.

12. The method according to claim 10, wherein changes in the score over time are associated to progression of the disease.

13. The method according to claim 10, wherein the classification model is developed using a supervised machine learning method.

14. The method according to claim 1, wherein the sample is a biofluid selected from the group consisting of blood, plasma, saliva, cerebrospinal fluid, brain sample, skin sample and urine.

15. A computer-implemented method for the identification of DLB in a subject, comprising:
(a) receiving data relating to the methylation pattern in the D-loop region and/or the ND1 gene of the mitochondrial DNA of a subject, wherein the methylation pattern is determined in at least one site selected from the group consisting of:
(i) the CpG sites in the D-loop region shown in Table 1,
(ii) the CHG sites in the D-loop region shown in Table 3,
(iii) the CHH sites in the D-loop region shown in Table 5,
(iv) the CpG sites of the ND1 gene shown in Table 2, and
(v) the CHG sites of the ND1 gene shown in Table 4,
and wherein the methylation pattern is determined in at least one of (vi) the CHH sites of the ND1 gene shown in Table 6,
and optionally at least one clinical variable of the subject, and
(b) determining a risk score correlating to the identification of DLB in a subject, wherein the risk score is calculated using a classification model configured to combine the methylation pattern of one or more sites of step (a) and optionally at least one clinical variable of the subject.
